(19) United States
(12) Patent Application Publication
(10) Pub. No.: US 2023/0142126 A1
(43) Pub. Date: May 11, 2023

(54) METHODS AND COMPOSITIONS FOR THE TREATMENT OF VIRAL DISEASES

(71) Applicant: HEALION BIO., INC., Frederick, MD (US)

(72) Inventors: Sina BAVARI, Frederick, MD (US); Simon NEWMAN, Frederick, MD (US)

(73) Assignee: HEALION BIO., INC., Frederick, MD (US)

(21) Appl. No.: 18/055,596

(22) Filed: Nov. 15, 2022

Related U.S. Application Data
(63) Continuation-in-part of application No. PCT/US2021/052664, filed on Sep. 29, 2021.
(63) Continuation-in-part of application No. PCT/US2021/032461, filed on May 14, 2021.
(60) Provisional application No. 63/084,754, filed on Sep. 29, 2020.
(60) Provisional application No. 63/188,723, filed on May 14, 2021.
(60) Provisional application No. 63/025,770, filed on May 15, 2020.

(51) Int. Cl. A61K 31/506; A61K 45/06; A61K 31/277; A61K 31/495; A61P 31/14

(52) U.S. Cl. CPC A61K 31/506; A61K 31/277; A61K 31/495; A61K 45/06; A61P 31/14

(57) ABSTRACT

The present disclosure relates to antiviral compositions and methods. Methods for using the antiviral compositions for inhibiting replication of viruses and treatment of viral diseases are also described. The present disclosure additionally relates to compositions and methods for enhancing a drug’s efficacy by combining the drug with a mammalian protease inhibitor such as a cathepsin inhibitor. Methods for using the combinations for treatment of disease are described.

### CROSS REFERENCE TO RELATED APPLICATIONS

This application is a Continuation-In-Part of International Application Number PCT/US2021/052664 filed on 29 Sep. 2021, which claims the benefit of U.S. Provisional Pat. Application No. 63/084,754, filed on 29 Sep. 2020 and U.S. Provisional Pat. Application No. 63/188,723 filed on 14 May 2021. This application is also a Continuation-In-Part of International Application Number PCT/US2021/032461 filed on 14 May 2021, which claims the benefit of U.S. Provisional Pat. Application No. 63/025,770, filed on 15 May 2020. All of the forgoing are incorporated by reference in their entireties.

### FIELD OF THE DISCLOSURE

The present disclosure provides protease inhibitor-based compositions and methods for the treatment of diseases such as diseases caused by virus.

The present disclosure also provides compositions and methods for increasing the effectiveness of an anti-viral composition against a disease when combined with a mammalian protease inhibitor e.g., a cathepsin inhibitor.

### BACKGROUND OF THE DISCLOSURE

The emergence of new viruses has exposed the need for innovative strategies to develop antiviral drugs. Severe acute respiratory syndrome coronavirus 2 (SARS-CoV-2) is an emergent coronavirus which causes a severe acute respiratory disease, COVID-19. There is an urgent need for preventive and therapeutic antiviral therapies and prophylaxis for SARS-CoV-2 and other viruses.

Repurposing previously identified drugs for therapeutic indications represents a potential path towards identifying promising candidate drugs to counteract current viral pathogens and possible emerging viruses.

During the drug development process, a vast number of candidate drugs are abandoned because of observed serious adverse reactions. In many cases, these adverse side effects are a result of the large doses required of the intrinsically toxic drug for therapeutic effect when the efficacy of the drug is low. An effective strategy for eliminating the toxicity of these drugs is to lower the required dosage by increasing the drug’s efficacy. Therefore, there is a need for a method for increasing the efficacy of a drug and reducing the required effective dose. The compositions and methods described herein meet this need.

### SUMMARY OF THE DISCLOSURE

The disclosure provides compositions of antiviral drug and/or mammalian protease inhibitors and methods. In some embodiments, the present disclosure provides a method of inhibiting the replication of a virus for example, a coronavirus. In some aspects, the methods can involve contacting the virus with a mammalian protease inhibitor. The method can further include measuring the replication of the virus. The replication of the virus can be measured by methods known in the art. In some embodiments, the present disclosure provides a method of reducing the percentage of virus infected cells in a population of cells. Such methods can include, contacting the virus infected cells with a mammalian protease inhibitor. The method further involves measuring the percentage of virus infected cells. Also provided herein are methods of reducing coronavirus infection in a subject. Such methods can include, contacting a subject in need with a mammalian protease inhibitor. The efficacy of the mammalian protease inhibitor in reducing the coronavirus infection can be measured after providing the subject with mammalian protease inhibitor.

In some embodiments, the mammalian protease inhibitor has the structure of Formula (I):

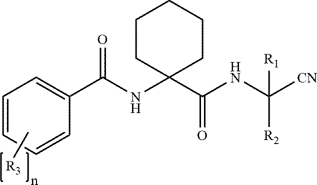

wherein, R1 and R2 are independently H or C1-C7 lower alkyl, or R1 and R2 together with the carbon atom to which they are attached form a C3-C8 cycloalkyl ring; and R3 is an optionally substituted heterocyclic group comprising at least one nitrogen; and n is between 1 and 3.

In some embodiments, the mammalian protease inhibitor can have the structure of Formula (II):

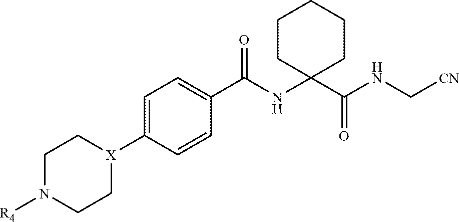

wherein X is CH or N; and [0010] R4 is H, C1-C7 lower alkyl, C1-C7 lower alkoxy, C5-C10 aryl, or C3-C8 cycloalkyl.

In one embodiment, the mammalian protease inhibitor can have the structure of

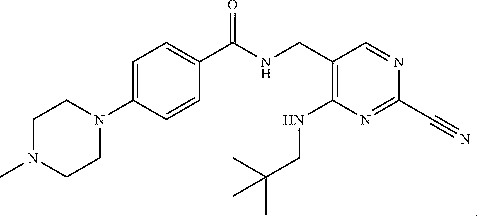

In one aspect, the mammalian protease inhibitor can have the structure of

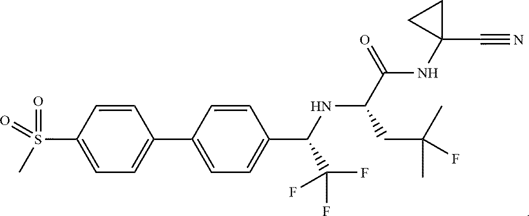

The mammalian protease inhibitor can be a cathepsin inhibitor. Non-limiting examples of cathepsin inhibitors include The method of claim 4, wherein the cysteine cathepsin inhibitor is Balicatib, E-64, E-64a, E-64b, E-64c, E- 64d, CA-074, CA-074 Me, CA-030, CA-028, peptidyl aldehyde derivatives leupeptin, antipain, chymostatin, Ac-LVK-CHO, Z-Phe-Tyr-CHO, a epoxisuccinate Z-Phe-Tyr(OtBu)-COCHO.H2O, 1-Naphthalenesulfonyl-Ile-Trp-CHO, Z-Phe-Leu-COCHO.H2O; peptidyl semicarbazone derivatives, peptidyl methylketone derivatives, peptidyl trifluoromethylketone, Biotin-Phe-Ala-fluoromethyl ketone, Z-Leu-Leu-Leu fluoromethyl ketone, Z-Phe-Phe-fluoromethyl ketone, N-Methoxysuccinyl-Phe-HOMO-Phe-fluoromethyl ketone, Z-Leu-Leu-Tyr-fluoromethyl ketone, Leupeptin trifluoroacetate, ketone, peptidylchloromethases peptidylhydroxymates, peptidylhydroxylamines, peptidyl acyloxymethanes, peptidylacyloxymethyl ketones, peptidyl aziridines, peptidyl aryl vinylsufones, peptidyl arylvinylsulfonates, gallinamide analogs, peptidyl aldehydes , azepinone-based inhibitors, nitrile-containing inhibitors, thiosemicarbazone , propeptide mimics, thiocarbazate, oxocarbazate, azapaptides, peptidyl halomethylketone derivatives, TLCK; bis(acylamino) ketone, 1,3-Bis(CBZ-Leu-NH)-2-propanone; peptidyl diazomethanes, Z-Phe-Ala-CHN2, Z-Phe-Thr(OBzl)-CHN2, Z-Phe-Tyr (Ot-But)-CHN2, Z-Leu-Leu-Tyr-CHN2; peptidyl acyloxymethyl ketones; peptidyl methylsulfonium salts; peptidyl vinyl sulfones, LHVS; peptidyl nitriles; peptidyl disulfides, 5,5′-dithiobis[2-nitrobenzoic acid], cysteamines, 2,2′-dipyridyl disulfide; N-(4-Biphenylacetyl)-S-methyl cysteine-(D)-Arg-Phe-b phenethylamide; thiol alkylating agents, maleimides, azapeptides, azobenzenes, O-acylhydroxamates, Z-Phe-Gly-NHO-Bz, Z-FG-NHO-BzOME, lysosomotropic agents, chloroquine, ammonium chloride, Cystatins A, Cystatin B, Cystatin C, Cystatin D, Cystatin F, stefins, kininogens, Sialostain L, antimicrobial peptide LL-37, Procathepsin B Fragment 26-50, Procathepsin B Fragment 36-50, Odanacatib (MK-0822), Relacatib (GSK-462795, SB-462795), SLV213 (K777 OR K1777), RO5459072, RWJ-445380, VBY036P1A, AM-3701, MIV-701, MIV-710, MIV-711, NC-2300, ORG-219517, ONO-5334,MK-0674, K777, GB-111-NH2, L-873724, L-006235, AZD4996, VBY-036, RWY-445380, AM-3840, Cz-007, VBY-825 (VBY-106; VBY-285;VBY-825), VBY-129, SAR-114137, VBY-891, Petesicatib (RG-7625; RO-5459072), LY-3000328, MIV-247, CRA-028129, RG-7236, GSK2793660, Aloxistatin (E-64d, Loxistatin, EST), BI-1181181 (VTP-37948),VBY-376, Aloxistatin (Ab-007; E-64-d), Begacestat (GSI-953; WAY-210953), AL101 (BMS906024), BMS-986115 (AL-102), MK-0752 (L-000891675), EVP-0962 (EVP-0015962), SAR-164653, KGP94, VEL-0230, and/or BLD2660. In one embodiment, the cathepsin inhibitor can be Balicatib, or a pharmaceutically acceptable salt or an ester of Balicatib.

In some embodiments, the virus can be a virus in the family, Coronaviridae, or a virus in the sub-family Orthocoronavirinae, or a virus in the order Nidovirales, In some embodiments, the methods of the disclosure can be used to inhibit the replication of any coronavirus. As a non-limiting example, the methods of the disclosure can be used to inhibit the replication of a coronavirus, such as a SARS-CoV-2 virus, SARS-CoV-1 virus, MERS-CoV virus, 229E virus, NL63 virus, OC43 virus, HKU1 virus, or variants thereof. As a non-limiting example, the coronavirus can be SARS-CoV-2 virus.

According to the methods of the present disclosure the concentration of the mammalian protease used can be from about 1 × 10⁻¹² M to about 1×10⁻³ M, for example, from about 0.1 µM to about 50 µM. In some embodiments, the effective concentration (EC₅₀) of the mammalian protease inhibitor against a coronavirus can be from about 0.25 µM to about 30 µM, for example, from about 0.5 µM to about 30 µM. In some embodiments, the effective concentration of the mammalian protease inhibitor against an enterovirus can be from about 15 µM to about 30 µM. The EC₅₀ of the mammalian protease inhibitor can be 0.1 µM, 0.3 µM, 1 µM, 3 µM, 10 µM or 30 µM. The EC₉₀ of the mammalian protease inhibitor can be from about 1 µM to 100 µM. As a non-limiting example, the EC₉₀ can be 1 µM to 100 µM. Contacting the coronavirus with the mammalian protease inhibitors can inhibit the replication of the coronavirus by from about 50% to about 100%. The mammalian protease inhibitor can be associated with a selectivity index of at least 300.

The disclosure relates to methods and combinations for increasing the efficacy of a drug in a subject such that the effective dosage is reduced thereby converting the drug into an effective therapeutic composition. In some embodiments, the subject can have a viral infection caused by a coronavirus e.g., SARS-CoV-2, SARS-CoV-1, or MERS-CoV.

The method can comprise combining a drug, such as an antiviral drug or an anticancer drug, with a mammalian protease inhibitor, for example a cathepsin inhibitor. In some embodiments, the drug effective concentration (EC) can be reduced through a potentiating, an additive or a synergistic activity when used in combination with a mammalian protease inhibitor such as a cathepsin inhibitor.

In one aspect, the present disclosure provides a method for improving the efficacy of a drug thereby reducing the effective dose and reducing toxic effects of the drug, said method comprising combining the drug with a mammalian protease inhibitor, for example a cathepsin inhibitor, such that the combination produces a potentiating, an additive or a synergistic activity enhancing the drug’s effectiveness and reducing the required effective dose or concentration. In some aspects, enhancing the efficacy of a drug by combining it with a mammalian protease inhibitor, for example a cathepsin inhibitor, can result in repurposing the drug for treatment of new indications not previously considered due to the initial low efficacy of the drug.

In another aspect, the present disclosure provides a method for lowering toxicity of a drug by reducing its effective concentration, comprising combining the drug with a mammalian protease inhibitor, for example a cathepsin inhibitor, such that a potentiating, an additive or a synergistic activity is created enhancing the drug’s effectiveness. In some aspects, lowering toxicity of a drug is achieved by combining it with a mammalian protease inhibitor, for example a cathepsin inhibitor, can result in repurposing the drug for treatment of new indications not previously considered.

In yet another aspect, the present disclosure provides a method for reducing the effective concentration of a drug in a subject, comprising administering said drug to said subject in combination with a mammalian protease inhibitor, for example a cathepsin inhibitor, such that the combination produces a potentiating, an additive or a synergistic activity thereby reducing the effective concentration of the drug. In some aspects, reducing the effective concentration of a drug by combining it with a mammalian protease inhibitor, for example a cathepsin inhibitor, can result in repurposing the drug for treatment of new indications not previously considered.

Therefore, the disclosure is related to a method and composition comprising a drug combination, the combination comprising a drug and a mammalian protease inhibitor, for example a cathepsin inhibitor, that reduces the effective concentration of the drug and therefore the intrinsic toxicity displayed by the drug. Depending on the situation, the drug and the mammalian protease inhibitor of this combination can be administered simultaneously, separately or sequentially. The administration of this combination can be performed by all possible routes including, e.g., systemic, topical, or oral routes. The combination can be used in the prophylaxis and/or treatment of disease by administering to a subject a therapeutically effective amount of the pharmaceutical combination.

In one aspect, the present disclosure provides a mixture or combination of a drug and a cathepsin inhibitor for use in the prophylaxis or treatment of disease. In another aspect, the present disclosure provides use of the mixture or combination of the disclosure for preparation of a medicament or a pharmaceutical preparation, whether provided as a combination in one formulation or provided as two separate compounds, whether by similar or different administration methods, concentrations, dosages, or schedules, for the prophylaxis or treatment of disease.

In another aspect, a drug and cathepsin inhibitor can be combined along with other cathepsin inhibitors, protease inhibitors, proteinase inhibitors or esterase inhibitors for example, a combination with cathepsin inhibitors such as Odanacatib (MK-0822), Balicatib (AAE581), Relacatib (GSK-462795, SB-462795), SLV213 (K777 OR K1777), RO5459072, RWJ-445380, VBY036P1A, AM-3701, MIV-701,MIV-710, MIV-711,NC- 2300, ORG-219517, ONO-5334, MK-0674, GB-111-NH2, L-873724, L-006235, AZD4996, VBY-036, RWY-445380, AM-3840, Cz-007, VBY-825 (VBY-106; VBY-285;VBY-825), VBY-129, SAR-114137, VBY-891, Petesicatib (RG-7625; RO-5459072), LY-3000328, MIV-247, CRA-028129, RG-7236, GSK2793660, Aloxistatin (E-64d, Loxistatin, EST), BI-1181181 (VTP-37948), VBY-376, Aloxistatin (Ab-007; E-64-d), Begacestat (GSI-953; WAY-210953), AL101 (BMS906024), BMS-986115 (AL-102), MK-0752 (L-000891675), EVP-0962 (EVP-0015962), BLD2660, SAR-164653, KGP94, and VEL-0230, combination with serine protease inhibitors such as Camostat, Odalasvir, Nafamostat mesylate, or any other protease inhibitors currently in use for HIV such as atazanavir (Reyataz), darunavir (Prezista), fosamprenavir (Lexiva), indinavir (Crixivan), lopinavir/ritonavir (Kaletra), nelfinavir (Viracept), ritonavir (Norvir), saquinavir (Invirase), tipranavir (Aptivus), atazanavir/cobicistat (Evotaz), darunavir/cobicistat (Prezcobix) or other protease inhibitors use for other viruses such as HCV for example asunaprevir, boceprevir, grazoprevir, glecaprevir, paritaprevir, simeprevir, telaprevir, or for use in HBV treatment.

In another aspect, a drug to be combined with a cathepsin inhibitor is an antiviral drug. In yet another aspect, the antiviral drug is a nucleoside analog, a nucleotide analog or a nucleic acid analog, a peptide, a synthetic small molecule, or a large molecule such as those obtained from plant and animal extracts.

In the case of nucleoside, nucleotide, and nucleic acid analogs, their use as antivirals has been hampered due to the high dosage required to produce the antiviral effect, resulting in increased off-target and toxicity in the subject. It was surprisingly discovered that administering a nucleoside analog in combination with a cathepsin inhibitor produced an unexpected synergistic effect, increasing the efficacy of the combination with reduced toxicity and reducing the required dosage of nucleoside analog.

In one aspect, the disclosure provides a composition comprising a mixture comprising an antiviral drug, for example a nucleotide or nucleoside analog, in combination with a cathepsin inhibitor. The present disclosure provides a method for increasing the efficacy of the antiviral drug by combining it with a cathepsin inhibitor such that the dosage of antiviral drug that must be administered for therapeutic effect is reduced to an amount that is safe and nontoxic.

In yet another aspect, the antiviral drug is a nucleoside analog or other antiviral, for example, BCX4430 (Galidesivir), T-705 (Avigan, Favipiravir), Brincidofovir, FGE-106, JK-05, Triazavirin, Acyclovir Fleximer, Ribavirin, AL-335 (Adafosbuvir), 6-azauridine, gancyclovir, dideocycytidine, dideoxyinosine, or resimiquid, remdesivir, gemcitabine hydrocholoride, mizoribine, lamivudine, entecavir, telbivudine, adefovir dipivoxil, tenofovir disoproxil fumarate (TDF), sofosbuvir, FV100 (FV for FermaVir), letermovir, daclatasvir, asunaprevir, beclabuvir, lopinavir, ritonavir, Hepsera (adefovir dipivoxil), Peveon, Viread (tenofovir disoproxil fumarate), Acycloadenosine (predecessor of acyclovir), NITD008, MK-608, ribonucleoside analog β-d-N4-hydroxycytidine (NHC; EIDD-1931), and EIDD-2801 (Molnupiravir), AT-527, and AT-511.

As a non-limiting example, the nucleoside analog can be T-705 (Avigan, Favipiravir).

In some embodiments, the antiviral drug can be a nucleotide analog.

In some embodiments, the antiviral drug can be a viral protease inhibitor. As a non-limiting example, the viral protease inhibitor can be PF-07321332, PF-07304814, PF-00835231, atazanavir (Reyataz), darunavir (Prezista), fosamprenavir (Lexiva), indinavir (Crixivan), lopinavir/ritonavir (Kaletra), nelfinavir (Viracept), ritonavir (Norvir), saquinavir (Invirase), tipranavir (Aptivus), atazanavir/cobicistat (Evotaz), darunavir/cobicistat (Prezcobix), asunaprevir, boceprevir, grazoprevir, glecaprevir, paritaprevir, simeprevir, or telaprevir.

In some embodiments, the antiviral drug can be a viral polymerase inhibitor. As a non-limiting example, the viral polymerase inhibitor can be Foscarnet, Cidofovir, or Alovudine.

In some embodiments, the antiviral drug can be a reverse transcriptase inhibitor. As a non-limiting example, the reverse transcriptase inhibitor can be Nevirapine, Delavirdine, Efavirenz, Etravirine, Etravirine, Rilpivirine, Adefovir dipivoxil, or Atevirdine.

In some embodiments, the antiviral drug can be a viral envelope fusion inhibitor. As a non-limiting example, the viral envelope fusion inhibitor can be Docosanol, Enfuvirtide, or Maraviroc.

In some embodiments, the antiviral drug can be a prophylactic agent such as RSV-IGIV, VZIG, or VariZIG.

In some embodiments, the antiviral drug can be an antibody.

In some embodiments, the antiviral drug can be a proton transport inhibitor. As a non-limiting example, the proton transport inhibitor can be Rimantadine or Methisazone.

In some embodiments, the antiviral drug can be a neuraminidase inhibitor. As a non-limiting example, the neuraminidase inhibitor can be Zanamivir, Oseltamivir, Laninamivir octanoate, or Peramivir.

In yet another aspect, the cathepsin inhibitor can be a cathepsin-B inhibitor, a cathepsin-L inhibitor, a cathepsin-S inhibitor, a cathepsin-F inhibitor, a cathepsin-X inhibitor, a cathepsin-K inhibitor, a cathepsin-V inhibitor, a cathepsin-W inhibitor, a cathepsin-C inhibitor, a cathepsin-O inhibitor, and a cathepsin-H inhibitor. In yet another aspect, the cathepsin inhibitor is a cathepsin-K inhibitor. In another aspect, the cathepsin inhibitor is epoxisuccinate and derivative thereof; or from E-64; E-64a; E-64b; E-64c; E-64d; CA-074; CA-074 Me; CA-030; CA-028; peptidyl aldehyde derivatives leupeptin, antipain, chymostatin, Ac-5 LVK-CHO, Z-Phe-Tyr-CHO, Z-Phe-Tyr(OtBu)-COCHO.H2O, 1-Naphthalenesulfonyl-Ile-Trp-CHO, Z-Phe-Leu-COCHO.H2O; peptidyl semicarbazone derivatives; peptidyl methylketone derivatives; peptidyl trifluoromethylketone derivatives Biotin-Phe-Ala fluoromethyl ketone, Z-Leu-Leu-Leu-fluoromethyl ketone, Z-Phe-Phe-fluoromethyl ketone, NMethoxysuccinyl-Phe-HOMO-Phe-fluoromethyl ketone, Z Leu- Leu-Tyr-fluoromethyl ketone, Leupeptin trifluoroacetate, ketone; peptidylchloromethases and derivatives thereof; peptidylhydroxymates and derivatives thereof; peptidylhydroxylamines and derivatives thereof; peptidyl acyloxymethanes and derivatives thereof; peptidylacyloxymethyl ketones and derivatives thereof; peptidyl aziridines and derivatives thereof; peptidyl aryl vinyl sulfones and derivatives thereof; peptidyl arylvinylsulfonates and derivatives thereof; gallinamide analogs and derivates thereof; peptidyl aldehydes and derivatives thereof; azepinone-based inhibitors and derivatives thereof; nitrile-containing inhibitors and derivates thereof; thiosemicarbazone and derivatives thereof; propeptide mimics and derivatives thereof; thiocarbazate, oxocarbazate, azapaptides and derivatives thereof; peptidyl halomethylketone derivatives, TLCK; bis(acylamino)ketone, 1,3- Bis(CBZ-Leu-NH)-2-propanone; peptidyl diazomethanes, Z-Phe-Ala-CHN2, Z-Phe-Thr(OBzl)-CHN2, Z-Phe-Tyr (Ot-But)-CHN2, Z-Leu-Leu-Tyr-CHN2; peptidyl acyloxymethyl ketones; peptidyl methylsulfonium salts; peptidyl vinyl sulfones, LHVS; peptidyl nitriles; peptidyl disulfides, 5,5′-dithiobis[2- nitrobenzoic acid], cysteamines, 2,2′-dipyridyl disulfide; non-covalent inhibitors, N-(4-Biphenylacetyl)-S-methyl cysteine-(D)-Arg-Phe-b- 5 phenethylamide; thiol alkylating agents, maleimides, etc; azapeptides; azobenzenes; O-acylhydroxamates, Z-Phe-Gly-NHO-Bz, Z-FG-NHO-BzOME; lysosomotropic agents, chloroquine, ammonium chloride; inhibitors based on Cystatins A, B, C, D, F, stefins, kininogens, Sialostain L, antimicrobial peptide LL-37, Procathepsin B Fragment 26-50, Procathepsin B Fragment 36-50; Odanacatib (MK-0822), Balicatib (AAE581), Relacatib (GSK-462795, SB-462795), SLV213 (K777 OR K1777), RO5459072, RWJ-445380, VBY036P1A, AM-3701, MIV-15 701,MIV-710, MIV-711,NC-2300, ORG-219517,ONO-5334, MK-0674, GB-111-NH2, L-873724, L-006235, AZD4996, VBY-036, RWY-445380, AM-3840, Cz-007, VBY-825 (VBY- 106; VBY-285;VBY-825), VBY-129, SAR-114137, VBY-891, Petesicatib (RG-7625; RO-5459072), LY-3000328, MIV-20 247, CRA-028129, RG-7236, GSK2793660, Aloxistatin (E-64d, Loxistatin, EST), BI-1181181 (VTP-37948), VBY-376, Aloxistatin (Ab-007; E-64-d), Begacestat (GSI-953; WAY-210953), AL101 (BMS906024), BMS-986115 (AL-102), MK-0752 (L-000891675), EVP-0962 (EVP-25 0015962), SAR-164653, VEL-0230, KGP94, and BLD2660.

In one aspect, the disclosure provides a composition and method for prophylaxis or treatment of a disease comprising administering in a pharmaceutically effective amount to a subject in need, e.g., a human patient, an antiviral drug, e.g., a nucleoside analog, in combination with a cathepsin inhibitor, such that the drug’s efficacy is increased, or a synergistic activity is produced, thereby reducing the effective dose of the drug along with its intrinsic toxicity.

In one aspect, the disease is a viral infection. In another aspect, the disease is one or more of cancer, tumor growth, sarcomas, metastasis, osteoporosis, osteoarthritis, atherosclerosis, hypertension, any endothelial related abnormality, systemic lupus erythemotosis, lupus nephritis, peripheral vascular disease, stroke, coronary heart disease, diabetes, insulin resistance, kidney failure, and/or venous thrombosis.

In another aspect, the viral infection is caused by a coronavirus, Orthomyxoviridae, influenza A virus, influenza B virus, influenza C virus, Thogotovirus, Dhori virus, infectious salmon anemia virus, Paramyxoviridae, parainfluenza virus, human respiratory syncytial virus (RSV), Sendai virus, Newcastle disease virus, mumps virus, rubeola (measles) virus, Hendra virus, Nipah virus, avian pneumovirus, canine distemper virus, Rhabdoviridae rabies virus, vesicular stomatitis virus (VSV), Mokola virus, Duvenhage virus, European bat virus, salmon infectious hematopoietic necrosis virus, viral hemorrhagic septicaemia virus, spring viremia of carp virus, and snakehead rhabdovirus, Bomaviridae, Boma disease virus, Bunyaviridae Bunyamwera virus, Hantaan virus, Crimean Congo virus, California encephalitis virus, Rift Valley fever virus, and sandfly fever virus, Arenaviridae Old World Arenaviruses, Lassa virus, Ippy virus, Lymphocytic choriomeningitis virus (LCMV), Mobala virus, Mopeia virus, New World Arenaviruses, Junin virus (Argentine hemorrhagic fever), Sabia (Brazilian hemorrhagic fever), Amapari virus, Flexalvirus, Guanarito virus (Venezuela hemorrhagic fever), Machupo virus (Bolivian hemorrhagic fever), Latino virus, Boliveros virus, Parana virus, Pichinde virus, Pirital virus, Tacaribe virus, Tamiami virus, Whitewater Arroyo virus, arboviruses togaviruses, Alphaviruses, Venezuela equine encephalitis virus, Sindbis virus, Rubivirus, Rubella virus, Flaviviridae, flavivirus, Pestivirus, and Hepacivirus, yellow fever virus, dengue fever virus, and Japanese encaphilitis (JE) virus, Pestivirus, Hepacivirus, hepatitis C virus, hepatitis C-like viruses, Japanese encephalitis Alfuy, Japanese encephalitis, Kokobera, Koutango, Kunjin, Murray Valley encephalitis, St. Louis encephalitis, Stratford, Usutu, West Nile viruses, Pestivirus, bovine viral diarrhea virus (BVDV), classical swine fever virus (CSFV), border disease virus (BDV), Arenaviridae, Lymphocytic choriomeningitis virus (LCMV), Lassa virus, Junin virus, Machupo virus, Guanarito virus, Sabia, Phlebovirus Rift valley fever virus, Hendra, Nipah, Riboviria, coronaviruses, SARS-CoV-1, SARS-CoV-2, and MERS-CoV.

In yet another aspect, the viral infection is caused by a coronavirus. In yet another aspect, the coronavirus is SARS-CoV, MERS-CoV, SARS-CoV-2, or any related viruses with positive or negative strand amplification with or without protective envelope.

In another aspect, the disclosure provides a composition and method for prophylaxis or treatment of a coronavirus infection such as SARS-CoV, MERSCoV, SARS-CoV-2, or any related viruses with positive or negative strand amplification with or without protective envelope, comprising administering in a pharmaceutically effective amount to a subject in need, e.g., a human patient, a cathepsin inhibitor in combination with an antiviral drug. In one aspect, the cathepsin inhibitor is a cathepsin-K inhibitor or a cathepsin-L inhibitor.

In another aspect, the disclosure provides a combination of one or more antiviral drugs and one or more cathepsin inhibitors for treatment of an viral infection, where the antiviral drug is one or more drugs such as, but not limited to, BCX4430 (Galidesivir), T-705 (Avigan, Favipiravir), Brincidofovir, FGE-106, JK-05, Triazavirin, Acyclovir Fleximer, Ribavirin, AL- 335 (Adafosbuvir), 6-azauridine, gancyclovir, dideocycytidine, dideoxyinosine, or resimiquid, remdesivir, gemcitabine hydrocholoride, mizoribine, lamivudine, entecavir, telbivudine, adefovir dipivoxil, tenofovir disoproxil fumarate (TDF), sofosbuvir, FV100 (FV for FermaVir), letermovir, daclatasvir, asunaprevir, beclabuvir, lopinavir, ritonavir, Hepsera (adefovir dipivoxil), Peveon, Viread (tenofovir disoproxil fumarate), Acycloadenosine (predecessor of acyclovir), NITD008, MK-608, ribonucleoside analog β-d-N4- hydroxycytidine (NHC; EIDD-1931), and EIDD-2801 (Molnupiravir), AT-527, and AT-511, and where the cathepsin inhibitor is one or more cathepsin inhibitor chosen from a cathepsin-B inhibitor, a cathepsin-L inhibitor, a cathepsin-S inhibitor, a cathepsin-F inhibitor, a cathepsin-X inhibitor, a cathepsin-K, inhibitor, a cathepsin-V inhibitor, a cathepsin-W inhibitor, a cathepsin-C inhibitor, a cathepsin-O inhibitor, and a cathepsin- H inhibitor. In yet another aspect, the cathepsin inhibitor is a cathepsin-K inhibitor. In another aspect, the cathepsin inhibitor is epoxisuccinate and derivative thereof; E-64; E-64a; E-64b; E-64c; E-64d; CA-074; CA-074 Me; CA-030; CA-028; peptidyl aldehyde derivatives leupeptin, antipain, chymostatin, Ac-LVK-CHO, Z-Phe-Tyr-CHO, Z-Phe- Tyr(OtBu)-COCHO.H2O, 1-Naphthalenesulfonyl-Ile-Trp- CHO, Z-Phe-Leu-COCHO.H2O; peptidyl semicarbazone derivatives; peptidyl methylketone derivatives; peptidyl trifluoromethylketone derivatives Biotin- Phe-Ala-fluoromethyl ketone, Z-Leu-Leu-Leu-fluoromethyl ketone, Z-Phe-Phe-fluoromethyl ketone, N-Methoxysuccinyl-Phe-HOMO-Phe-fluoromethyl ketone, Z-Leu-Leu-Tyr-fluoromethyl ketone, Leupeptin trifluoroacetate, ketone; peptidylchloromethases and derivatives thereof; peptidylhydroxymates and derivatives thereof; peptidylhydroxylamines and derivatives thereof; peptidyl acyloxymethanes and derivatives thereof; peptidylacyloxymethyl ketones and derivatives thereof; peptidyl aziridines and derivatives thereof; peptidyl aryl vinyl sulfones and derivatives thereof; peptidyl arylvinylsulfonates and derivatives thereof; gallinamide analogs and derivates thereof; peptidyl aldehydes and derivatives thereof; azepinone-based inhibitors and derivatives thereof; nitrile-containing inhibitors and derivates thereof; thiosemicarbazone and derivatives thereof; propeptide mimics and derivatives thereof; thiocarbazate, oxocarbazate, azapaptides and derivatives thereof; peptidyl halomethylketone derivatives, TLCK; bis(acylamino)ketone, 1,3-Bis(CBZ-Leu-NH)-2-propanone; peptidyl diazomethanes, Z-Phe-Ala-CHN2, Z-Phe-Thr(OBzl)-CHN2, Z-Phe-Tyr (Ot- But)-CHN2, Z-Leu-Leu-Tyr-CHN2; peptidyl acyloxymethyl ketones; peptidyl methylsulfonium salts; peptidyl vinyl sulfones, LHVS; peptidyl nitriles; peptidyl disulfides, 5,5′-dithiobis[2-nitrobenzoic acid], cysteamines, 2,2′-dipyridyl disulfide; non-covalent inhibitors, N-(4-10 Biphenyl acetyl)-S-methyl cysteine-(D)-Arg-Phe-b-phenethylamide; thiol alkylating agents, maleimides, etc; azapeptides; azobenzenes; O-acylhydroxamates, Z-Phe-Gly-NHO-Bz, Z-FG-NHO-BzOME; lysosomotropic agents, chloroquine, ammonium chloride; inhibitors based on Cystatins A, B, C, D, F, stefins, kininogens, Sialostain L, antimicrobial peptide LL-37, Procathepsin B Fragment 26-50, Procathepsin B Fragment 36-50; Odanacatib (MK-0822), Balicatib (AAE581), Relacatib (GSK-462795, SB-462795), SLV213 (K777 OR K1777), ,RO5459072, RWJ-445380, VBY036P1A, AM-3701, MIV- 701,MIV-710, MIV-711,NC-2300, ORG-219517,ONO-5334, MK-0674, GB-111-NH2, L-873724, L-006235, AZD4996, VBY-036, RWY-445380, AM-3840, Cz-007, VBY-825 (VBY- 106; VBY-285;VBY-825), VBY-129, SAR-114137, VBY-891, Petesicatib (RG-7625; RO-5459072), LY-3000328, MIV-247, CRA-028129, RG-7236, GSK2793660, Aloxistatin (E-64d, Loxistatin, EST), BI-1181181 (VTP-37948), VBY-376, Aloxistatin (Ab-007; E-64-d), Begacestat (GSI-953; WAY-210953), AL101 (BMS906024), BMS-986115 (AL-102), MK-0752 (L-000891675), EVP-0962 (EVP-0015962), SAR-164653, VEL-0230, KGP94, and BLD2660.

In another aspect, the present disclosure provides a composition and method for the treatment of viral infection, the composition comprising one or more cathepsin inhibitors chosen from a cathepsin-B inhibitor, a cathepsin-L inhibitor, a cathepsin-S inhibitor, a cathepsin-F inhibitor, a cathepsin-X inhibitor, a cathepsin-K, inhibitor, a cathepsin-V inhibitor, a cathepsin-W inhibitor, a cathepsin-C inhibitor, a cathepsin-O inhibitor, and a cathepsin-H inhibitor. In yet another aspect, the cathepsin inhibitor is a cathepsin-K inhibitor. In another aspect, the cathepsin inhibitor is epoxisuccinate and derivative thereof; E-64; E-64a; E-64b; E-64c; E-64d; CA-074; CA-074 Me; CA-030; CA-028; peptidyl aldehyde derivatives leupeptin, antipain, chymostatin, Ac-LVK-CHO, Z-Phe-Tyr-CHO, Z-Phe-Tyr(OtBu)-COCHO.H2O, 1-Naphthalenesulfonyl-Ile-Trp-CHO, Z-Phe-Leu-COCHO.H2O; peptidyl semicarbazone derivatives; peptidyl methylketone derivatives; peptidyl trifluoromethylketone derivatives Biotin-Phe-Ala-fluoromethyl ketone, Z-Leu-Leu-Leu-fluoromethyl ketone minimum, Z-Phe-Phe-fluoromethyl ketone, N25 Methoxysuccinyl-Phe-HOMO-Phe-fluoromethyl ketone, ZLeu- Leu-Tyr-fluoromethyl ketone, Leupeptin trifluoroacetate, ketone; peptidylchloromethases and derivatives thereof; peptidylhydroxymates and derivatives thereof; peptidylhydroxylamines and derivatives thereof; peptidyl acyloxymethanes and derivatives thereof; peptidylacyloxymethyl ketones and derivatives thereof; peptidyl aziridines and derivatives thereof; peptidyl aryl vinyl sulfones and derivatives thereof; peptidyl arylvinylsulfonates and derivatives thereof; gallinamide analogs and derivates thereof; peptidyl aldehydes and derivatives thereof; azepinone-based inhibitors and derivatives thereof; nitrile-containing inhibitors and derivates thereof; thiosemicarbazone and derivatives thereof; propeptide mimics and derivatives thereof; thiocarbazate, oxocarbazate, azapaptides and derivatives thereof; peptidyl halomethylketone derivatives, TLCK; bis(acylamino)ketone, 1,3-Bis(CBZ-Leu-NH)-2-propanone; peptidyl diazomethanes, Z-Phe-Ala-CHN2, Z-Phe-Thr(OBzl)-CHN2, Z-Phe-Tyr (Ot-But)-CHN2, Z-Leu-Leu-Tyr-CHN2; peptidyl acyloxymethyl ketones; peptidyl methylsulfonium salts; peptidyl vinyl sulfones, LHVS; peptidyl nitriles; peptidyl disulfides, 5,5′-dithiobis[2-nitrobenzoic acid], cysteamines, 2,2′-dipyridyl disulfide; non-covalent inhibitors, N-(4-Biphenylacetyl)-S-methylcysteine-(D)-Arg-Phe-bphenethylamide; thiol alkylating agents, maleimides, etc; azapeptides; azobenzenes; O-acylhydroxamates, Z-Phe-Gly-NHO-Bz, Z-FG-NHO-BzOME; lysosomotropic agents, chloroquine, ammonium chloride; inhibitors based on Cystatins A, B, C, D, F, stefins, kininogens, Sialostain L, antimicrobial peptide LL-37, Procathepsin B Fragment 26-50, Procathepsin B Fragment 36-50; Odanacatib (MK-0822), Balicatib (AAE581), Relacatib (GSK-462795, SB-462795), SLV213 (K777 or K1777), RO5459072, RWJ-445380, VBY036P1A, AM-3701, MIV-701,MIV-710, MIV-711,NC-2300, ORG-219517,ONO-5334, MK-0674, GB-111-NH2, L-873724, L-006235, AZD4996, 30 VBY-036, RWY-445380, AM-3840, Cz-007, VBY-825 (VBY-106; VBY-285;VBY-825), VBY-129, SAR-114137, VBY-891, Petesicatib (RG-7625; RO-5459072), LY-3000328, MIV-247, CRA-028129, RG-7236, GSK2793660, Aloxistatin (E-64d, Loxistatin, EST), BI-1181181 (VTP-37948), VBY-376, Aloxistatin (Ab-007; E-64-d), Begacestat (GSI-953; WAY-210953), AL101 (BMS906024), BMS-986115 (AL-102), MK-0752 (L-000891675), EVP-0962 (EVP-0015962), KGP94, SAR-164653, and VEL-0230, and BLD2660. Examples of other cathepsin inhibitors are described in Dana et al., 2020. A Review of Small Molecule Inhibitors and Functional Probes of Human Cathepsin L. Molecules 25: 698. All references cited are herein incorporated in their entirety by reference thereto.

### BRIEF DESCRIPTION OF THE DRAWINGS

The foregoing and other objects, features, and advantages will be apparent from the following description of particular embodiments of the disclosure, as illustrated in the accompanying drawings. The drawings are not necessarily to scale, emphasis instead being placed upon illustrating the principles of various embodiments of the disclosure.

FIG. 1 shows the percentage of virus infected cells treated with varying concentrations of Balicatib (HB-121).

FIG. 2 shows the percentage of virus infected cells treated with varying concentrations of ONO-5334.

FIG. 3 shows the percentage of virus infected cells treated with varying concentrations of Odanacatib (MK-0822).

FIG. 4 is a graph depicting an effect of adding a cathepsin inhibitor to a weak antiviral drug. The cathepsin inhibitor was able to enhance the antiviral activity of the drug by more than a log. The large arrow depicts the shift in the Effective Concentration (EC₅₀) of the antiviral. The vertical dotted line depicts cytotoxicity of the drug with or without a potentiator.

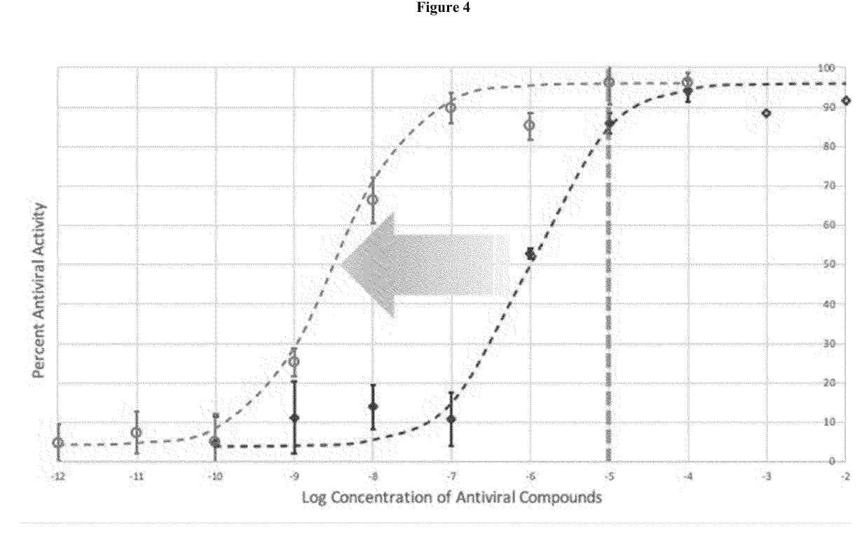

FIG. 5 shows TCID₅₀ of WA1/2020-infected A549-hACE2 cells after 24 hour incubation with each concentration of Balicatib (HB-121). In FIG. 5, LOD indicates limit of detection. The black dotted line shows the level of WA1/2020 virus without Balicatib (HB-121).

### DETAILED DESCRIPTION OF THE DISCLOSURE

### I. Introduction

Viruses comprise a large group of pathogens that are responsible for causing severe infectious diseases. Therapeutic agents targeting viruses can be broadly classified into (i) therapeutic agents that target the viruses themselves or (ii) therapeutic agents that target host cell factors.

Virus-targeting therapeutic agents can function directly or indirectly to inhibit the biological functions of viral proteins, such as enzymatic activities, or to block viral replication machinery. Host-targeting therapeutic agents target the host proteins that play a role in the viral life cycle, regulating the function of the immune system or other cellular processes in host cells. In some embodiments, the present disclosure provides host-targeting therapeutic agents for the treatment of viral diseases. In some embodiments, the present disclosure provides virus-targeting therapeutic agents and the related compositions. Also provided herein are methods of inhibiting the replication of a virus, and methods of reducing the percentage of virus infected cells in a population of cells.

During the drug development process, a vast number of candidate drugs are abandoned because of observed serious adverse reactions. In many cases, these adverse side effects are a result of the large doses required of the intrinsically toxic drug for therapeutic effect when the efficacy of the drug is low. An effective strategy for eliminating the toxicity of these drugs is to lower the required dosage by increasing the drug’s efficacy. Therefore, there is a need for a method for increasing the efficacy of a drug and reducing the required effective dose. The compositions and methods described herein meet this need.

Provided herein are compositions comprising a drug and/or a mammalian protease inhibitor. In some embodiments, the combination of the mammalian protease inhibitor with the drug produces a synergistic activity. The synergistic activity of the mammalian protease inhibitor with the drug can result in a reduction of the effective dose of the drug required to produce a desired effect. The reduced dose can in turn can lower the toxicity associated with the drug. In some embodiments, the drug is an antiviral drug.

The present disclosure provides methods wherein a mammalian protease inhibitor increases the bioavailability of a drug combined with the mammalian protease inhibitor, for example a cathepsin inhibitor, as measured by AUC of at least 25% relative to dosing of the drug alone. The present disclosure also provides methods wherein the mammalian protease inhibitor can increase bioavailability of the drug in the combination as measured by AUC of at least 50% relative to dosing of the drug alone. The present disclosure further provides methods wherein the mammalian protease inhibitor can increase the bioavailability of the drug in combination as measured by AUC of at least 100% relative to dosing of the drug alone. The disclosure provides mammalian protease inhibitors that can increase the bioavailability of the drug when used in combination, for example a cathepsin inhibitor, as measured by Cₘₐₓ of at least 50% relative to dosing of the drug alone. Changes in the integrated systemic concentrations over time are indicated by area under the curve (AUC) or Cₘₐₓ, both parameters well known in the art. AUC is a determination of the Area Under the Curve plotting the serum or plasma concentration of drug along the ordinate (Y-axis) against time along the abscissa (X-axis). Generally, the values for the AUC represent drug concentrations over time in units of mass-time/volume. When efficacy of a drug is being measured, the amount and form of the drug administered should be the same in both the administration of the drug in combination with the mammalian protease inhibitor, for example a cathepsin inhibitor, or the administration of the drug alone.

The disclosure also provides mammalian protease inhibitors that increase the bioavailability of the drug e.g., an antiviral drug when used in combination with the mammalian protease inhibitor, as measured by Cₘₐₓ of at about 90 to 110% relative to dosing of the drug alone. The disclosure further provides mammalian protease inhibitors that provide an increase in bioavailability of the drug when used in combination as measured by Cₘₐₓ of at least 110%, 120%, 130%, 140%, 150%, 160%, 170%, 180%, 190%, 200%, 300%, or more relative to dosing of drug alone. Systemic drug concentrations are measured using standard biochemical drug measurement techniques (Simmons et al., Anal Lett. 39: 2009-2021 (1997); the contents of which are herein incorporated by reference in its entirety).

The present disclosure provides mammalian protease inhibitors that increase clearance of the drug when used in combination with the mammalian protease inhibitor, for example a cathepsin inhibitor, from normal tissues as measured by pharmacological studies of at least 5%, 10%, 15%, 20%, 25%, 30%, 35%, 40%, 45%, 50%, 55%, 60%, 65%, 70%, 75%, 80%, 85%, 90%, 95% or more relative to dosing of the drug alone Clearance of drug normally occurs from the liver and kidneys and it is assumed that only free and not protein bound, drug is available for clearance. For hepatic clearance, passive diffusion through the lipid core of the hepatocyte membranes, available to lipophilic drugs, is augmented by sinusoidal carrier systems particularly for ionized molecules (anionic and cationic) of molecular weights of approximately 3-400. Likewise other transporters on the canalicular face transport drugs or their metabolites into bile. This system has two separate processes, hepatic uptake and biliary excretion. With small sized lipophilic drugs that readily traverse membranes hepatic uptake is not a maj or factor, but with higher molecular weight compounds (above 500) and those containing considerable H-bonding hepatic uptake can become the key clearance process, even if metabolism occurs subsequent to this.

When a drug rapidly dissolves and readily crosses the intestinal membranes, absorption tends to be complete, but absorption of orally administered drugs is not always complete. Before reaching the vena cava, a drug must move down the gastrointestinal tract and pass through the gut wall and liver, common sites of drug metabolism. Thus, a drug can be metabolized during first-pass metabolism before it can be measured in the systemic circulation. Many drugs have low oral bioavailability because of expensive first-pass metabolism.

Low bioavailability is most common with oral dosage forms of poorly water-soluble, slowly absorbed drugs. More factors can affect bioavailability when absorption is slow or incomplete than when it is rapid and complete. That is, slow or incomplete absorption leads to variable therapeutic responses. Slow absorption in the gastrointestinal tract also leads to increased acute and delayed-phase chemotherapy induced nausea and vomiting.

Insufficient time in the gastrointestinal tract is a common cause of low bioavailability. Ingested drug is exposed to the entire gastrointestinal tract for no more than one to two days and to the small intestine for only 2 to 4 hours. If the drug does not dissolve readily or cannot penetrate the epithelial membrane (e.g., if it is highly ionized and polar), time at the absorption site can be insufficient. In such cases, bioavailability tends to be highly variable as well as low. Age, sex, activity, genetic phenotype, stress, disease or previous gastrointestinal surgery can affect drug bioavailability.

Reactions that compete with absorption can reduce bioavailability. They include complex formation, hydrolysis by gastric acid or digestive enzymes, conjugation in the gut wall, absorption of other drugs and metabolism by luminal micro flora.

Assessment of bioavailability from plasma concentration-time data usually involves determining maximum peak concentration, the time at which maximum peak plasma drug concentration occurs, and the area under the plasma concentration time curve (AUC). The plasma drug concentration increases with the extent of absorption. The peak is reached when the drug elimination rate equals absorption rate. AUC is the most reliable measure of bioavailability. It is directly proportional to the total amount of unchanged drug that reaches the systemic circulation.

Drug products can be considered bioequivalent in extent and rate of absorption if their plasma level curves are essentially super imposable. Drug products that have similar AUCs but differently shaped plasma level curves are equivalent in extent but differ in their absorption rate-time profiles. Absorption occurs by one of three methods, either passive diffusion, active transport, or facilitated active transport. Passive diffusion is simply the passage of molecules across the mucosal barrier until the concentration of molecules reaches osmotic balance on both sides of the membrane. In active transport the molecule is actively pumped across the mucosa. In facilitated transport, a carrier generally a protein, is required to convey the molecule across the membrane for absorption.

### II. Compositions

Compositions as used herein can include a drug, e.g., an antiviral drug and a mammalian protease inhibitor.

The word “drug” as used herein is defined as a chemical intended for use in the treatment or prevention of disease. For clarity, as used herein, the term drug does not encompass mammalian protease inhibitors described herein. Drugs include synthetic and naturally occurring bio affecting substances as well as recognized pharmaceuticals, such as those listed in “The Physician desk Reference,” 56th ed, pages 101-133 (or an updated edition). These references are incorporated by reference herein. The term “drug” also includes similar drugs that have the indicated properties that are not discovered or available. The present disclosure provides drugs comprising charged, uncharged, hydrophilic, zwitter-ionic, or hydrophobic species, as well as any combinations of these physical characteristics. A hydrophobic drug is defined as a drug which in its non-ionized form is more soluble in lipid or fat than in water. One class of hydrophobic drugs includes those that are more soluble in octanol than in water. In some embodiments, drugs can be antiviral drugs.

### Protease Inhibitors

In some embodiments, the compositions of the disclosure can include protease inhibitors. Protease inhibitors are small molecules that block or reduce the activity of a protease. In some embodiments, proteases can be essential for virus replication. Many human pathogenic viruses use human enzymes to activate the viral proteins and successfully overtake the infected cell processes. For example, human cathepsins assist in the cleavage of viral proteins that are essential for the virus life cycle. These proteases can include, but are not limited to cysteine proteases, serine proteases, aspartic proteases.

In some embodiments, the compositions of the disclosure may be or may include a “cathepsin inhibitor” which, as used herein may refer to an agent which is capable of reducing, suppressing, or inhibiting the activity of the class of endosomal proteases called cathepsins. In some embodiments, the cathepsins can require acidic pH for enzyme activity. In other aspects, the cathepsins can be enzymatically active at neutral pH. There are approximately a dozen members of this family, which are distinguished by their structure, catalytic mechanism, and which proteins they cleave. Cathepsins have a vital role in mammalian cellular turnover, e.g. bone resorption. They degrade polypeptides and are distinguished by their substrate specificities.

Most C1 cysteine cathepsins are endopeptidases (L, S, K, V, F), while cathepsin X is a carboxypeptidase and cathepsins B, C and H have both endopeptidase- and exopeptidase activities. The substrate-binding region of cysteine cathepsins is defined as an arrangement of binding subsites (SeS0) for peptide substrate amino acids (PeP0) on both sides (N- and C-) of the scissile bond, encompassing the stretch of seven sites from S4 to S30 of papain. Since the crystal structure of numerous substrate analogue inhibitors are available, the definition has been revised and redefined, limiting the binding of substrate residues to subsites S2eS 10, in which both main-chain and side-chain atoms are involved. However recent studies have shown the importance of cathepsin K site S3 for determining substrate specificity. Whereas the S2 binding site is a true deep pocket, the other sites provide a binding surface. Furthermore, while S2 and S10 sites are the major determinants of specificity, S1 is important for the affinity and efficient catalysis of substrates. The positioning of the P3 residue in site S3 is, as in subsite S20, mediated only by side chain contacts over a relative wide area. Cathepsins K, L, S and V have somewhat overlapping specificities, making it difficult to discriminate between them in vivo. Cathepsin K attacks sites having aliphatic amino acids (Leu, Ile, Val), unlike cathepsins L and V (which both rather accept hydrophobic residues with preference for Phe), and also accommodates Pro in the S2 subsite. Cathepsin K is unusual among cysteine cathepsins in that it can cleave substrates with Pro in the P2 position, although it has been reported that congopain, a cysteine protease from Trypanosoma congolense, with an amino acid sequence (65% of homology) and biochemical properties similar to cathepsin K, also does so. Another feature of cathepsin K is its preference for Gly at the P3 position.

Cathepsin K is a protease, which is defined by its high specificity for kinins, that are involved in bone resorption. The enzyme’s ability to catabolize elastin, collagen, and gelatin allow it to break down bone and cartilage. This catabolic activity is also partially responsible for the loss of lung elasticity and recoil in emphysema. Cathepsin K inhibitors, such as odanacatib, show great potential in the treatment of osteoporosis. Cathepsin K is also expressed in a significant fraction of human breast cancers, where it could contribute to tumor invasiveness. Mutations in this gene are the cause of pycnodysostosis, an autosomal recessive disease characterized by osteosclerosis and short stature. Cathepsin K expression is stimulated by inflammatory cytokines that are released after tissue injury.

Proteases can be grouped according to the key catalytic group in the active site. For example, the active site of the protease can include a serine (Ser), a threonine (Thr), a cysteine (Cys), an aspartate (Asp), a glutamate (Glu), or a zinc in the case of metalloproteases. Accordingly, the proteases can be a serine protease, a threonine protease, a cysteine protease, an aspartate protease, a glutamate protease, or a zinc protease.

In some embodiments, the compositions of the disclosure can include a protease inhibitor. As used herein a protease inhibitor is a molecule that blocks or reduces the activity of a protease. Proteases are essential for virus replication. Many human pathogenic viruses use human enzymes to activate the viral proteins and successfully overtake the infected cell processes.

Proteases can be grouped according to the key catalytic group in the active site. For example, the active site of the protease can include a serine (Ser), a threonine (Thr), a cysteine (Cys), an aspartate (Asp), a glutamate (Glu), or a zinc in the case of metalloproteases. Accordingly, the proteases can be a serine protease, a threonine protease, a cysteine protease, an aspartate protease, a glutamate protease, or a zinc protease.

In one embodiment, the protease can be a mammalian protease and the inhibitor can be a mammalian protease inhibitor. In aspect, the mammalian protease can be a cathepsin protease and the inhibitor can be a cathepsin protease inhibitor.

Human cathepsins assist in the cleavage of viral proteins that are essential for the virus life cycle. These proteases include, but are not limited to, cysteine proteases and proteinases, serine proteases, and aspartic proteases.

In some embodiments, the mammalian protease inhibitor can be a cathepsin inhibitor. As used herein, a “cathepsin inhibitor” is an agent whose pharmacological effect is to inhibit the activity of the class of endosomal cysteine proteases that require acidic pH for enzyme activity. In some embodiments, the cathepsin can be a cysteine protease and is herein referred to as a cysteine cathepsin. Examples of human cysteine cathepsin proteases include, but are not limited to, cathepsins, which include but are not limited to cathepsin B, cathepsin L, cathepsin S, cathepsin F, cathepsin X, cathepsin K, cathepsin V, cathepsin W, cathepsin C, cathepsin O, and cathepsin H. Cathepsin inhibitors useful in non-human animals are often categorized differently but are known to those of skill in the art. Thus, the inhibitors include cathepsin inhibitors which are known to correspond with human cathepsin inhibitors. Inhibitors of these cathepsins are useful according to methods of the disclosure.

In one embodiment of the disclosure, the cysteine protease inhibitor is a cathepsin inhibitor such as a cathepsin-B inhibitor, a cathepsin-L inhibitor, a cathepsin-S inhibitor, a cathepsin-F inhibitor, a cathepsin-X inhibitor, a cathepsin-K, inhibitor, a cathepsin-V inhibitor, a cathepsin-W inhibitor, a cathepsin-C inhibitor, a cathepsin-O inhibitor, and a cathepsin-H inhibitor. In yet another aspect, the cathepsin inhibitor is a cathepsin-K inhibitor. In another aspect, the cathepsin inhibitor is epoxisuccinate and derivative thereof; E-64; E-64a; E-64b; E-64c; E-64d; CA-074; CA-074 Me; CA-030; CA-028; peptidyl aldehyde derivatives leupeptin, antipain, chymostatin, Ac-LVK-CHO, Z-Phe-Tyr-CHO, Z-Phe-Tyr(OtBu)-COCHO.H2O, 1-Naphthalenesulfonyl-Ile-Trp- CHO, Z-Phe-Leu-COCHO.H2O; peptidyl semicarbazone derivatives; peptidyl methylketone derivatives; peptidyl trifluoromethylketone derivatives Biotin- Phe-Ala-fluoromethyl ketone, Z-Leu-Leu-Leu-fluoromethyl ketone, Z-Phe-Phe-fluoromethyl ketone, N-Methoxysuccinyl-Phe-HOMO-Phe-fluoromethyl ketone, Z-Leu-Leu-Tyr-fluoromethyl ketone, Leupeptin trifluoroacetate, ketone; peptidylchloromethases and derivatives thereof; peptidylhydroxymates and derivatives thereof; peptidylhydroxylamines and derivatives thereof; peptidyl acyloxymethanes and derivatives thereof; peptidylacyloxymethyl ketones and derivatives thereof; peptidyl aziridines and derivatives thereof; peptidyl aryl vinyl sulfones and derivatives thereof; peptidyl arylvinylsulfonates and derivatives thereof; gallinamide analogs and derivates thereof; peptidyl aldehydes and derivatives thereof; azepinone-based inhibitors and derivatives thereof; nitrile-containing inhibitors and derivates thereof; thiosemicarbazone and derivatives thereof; propeptide mimics and derivatives thereof; thiocarbazate, oxocarbazate, azapaptides and derivatives thereof; peptidyl halomethylketone derivatives, TLCK; bis(acylamino)ketone, 1,3- Bis(CBZ-Leu-NH)-2-propanone; peptidyl diazomethanes, Z-Phe-Ala-CHN2, Z-Phe-Thr(OBzl)-CHN2, Z-Phe-Tyr (Ot- But)-CHN2, Z-Leu-Leu-Tyr-CHN2; peptidyl acyloxymethyl ketones; peptidyl methylsulfonium salts; peptidyl vinyl sulfones, LHVS; peptidyl nitriles; peptidyl disulfides, 5,5′-dithiobis[2- nitrobenzoic acid], cysteamines, 2,2′-dipyridyl disulfide; non-covalent inhibitors, N-(4- Biphenyl acetyl)-S-methyl cysteine-(D)-Arg-Phe-b-phenethylamide; thiol alkylating agents, maleimides, etc; azapeptides; azobenzenes; O-acylhydroxamates, Z-Phe-Gly-NHO-Bz, Z-FG-NHO-BzOME; lysosomotropic agents, chloroquine, ammonium chloride; Cystatin A, Cystatin B, Cystatin C, Cystatin D, Cystatin F, stefins, kininogens, Sialostain L, antimicrobial peptide LL- 37, Procathepsin B Fragment 26-50, Procathepsin B Fragment 36-50; Odanacatib (MK-0822), Balicatib (AAE581), Relacatib (GSK-462795, SB-462795), SLV213 (K777 or K1777), RO5459072, RWJ-445380, VBY036P1A, AM-3701, MIV- 701, MIV-710, MIV-711, NC-2300, ORG-219517, ONO-5334, MK-0674, GB-111-NH2, L-873724, L-006235, AZD4996, VBY-036, RWY-445380, AM-3840, Cz-007, VBY-825 (VBY- 106; VBY-285;VBY-825), VBY-129, SAR-114137, VBY-891, Petesicatib (RG-7625; RO-5459072), LY-3000328, MIV32 247, CRA-028129, RG-7236, GSK2793660, Aloxistatin (E-64d, Loxistatin, EST), BI-1181181 (VTP-37948), VBY-376, Aloxistatin (Ab-007; E-64-d), Begacestat (GSI-953; WAY-210953), AL101 (BMS906024), BMS-986115 5 (AL-102), MK-0752 (L-000891675), EVP-0962 (EVP- 0015962), SAR-164653, KGP94, VEL-0230, and BLD2660.

Other cathepsin inhibitors are described in Dana et al., 2020. A Review of Small Molecule Inhibitors and Functional Probes of Human Cathepsin L. Molecules 10 25: 698, incorporated herein by reference thereto and can be useful in the present disclosure.

### Mammalian Protease Inhibitors

In one embodiment, the protease can be a mammalian protease and the inhibitor can be a mammalian protease inhibitor. In one aspect, the mammalian protease can be a cathepsin protease and the inhibitor can be a cathepsin protease inhibitor.

In one embodiment of the disclosure, the cysteine protease inhibitor is a cathepsin inhibitor such as a cathepsin-B inhibitor, a cathepsin-L inhibitor, a cathepsin-S inhibitor, a cathepsin-F inhibitor, a cathepsin-X inhibitor, a cathepsin-K, inhibitor, a cathepsin-V inhibitor, a cathepsin-W inhibitor, a cathepsin-C inhibitor, a cathepsin-O inhibitor, and a cathepsin-H inhibitor. In yet another aspect, the cathepsin inhibitor is a cathepsin-K inhibitor.

In another aspect, the cathepsin inhibitor is epoxisuccinate and derivative thereof; E-64; E-64a; E-64b; E-64c; E-64d; CA-074; CA-074 Me; CA-030; CA-028; peptidyl aldehyde derivatives leupeptin, antipain, chymostatin, Ac-LVK-CHO, Z-Phe-Tyr-CHO, Z-Phe-Tyr(OtBu)-COCHO.H2O, 1-Naphthalenesulfonyl-Ile-Trp-CHO, Z-Phe-Leu-COCHO.H2O; peptidyl semicarbazone derivatives; peptidyl methylketone derivatives; peptidyl trifluoromethylketone derivatives Biotin- Phe-Ala-fluoromethyl ketone, Z-Leu-Leu-Leu-fluoromethyl ketone, Z-Phe-Phe-fluoromethyl ketone, N-Methoxysuccinyl-Phe-HOMO-Phe-fluoromethyl ketone, Z-Leu-Leu-Tyr-fluoromethyl ketone, Leupeptin trifluoroacetate, ketone; peptidylchloromethases and derivatives thereof; peptidylhydroxymates and derivatives thereof; peptidylhydroxylamines and derivatives thereof; peptidyl acyloxymethanes and derivatives thereof; peptidylacyloxymethyl ketones and derivatives thereof; peptidyl aziridines and derivatives thereof; peptidyl aryl vinyl sulfones and derivatives thereof; peptidyl arylvinylsulfonates and derivatives thereof; gallinamide analogs and derivates thereof; peptidyl aldehydes and derivatives thereof; azepinone-based inhibitors and derivatives thereof; nitrile-containing inhibitors and derivates thereof; thiosemicarbazone and derivatives thereof; propeptide mimics and derivatives thereof; thiocarbazate, oxocarbazate, azapaptides and derivatives thereof; peptidyl halomethylketone derivatives, TLCK; bis(acylamino)ketone, 1,3- Bis(CBZ-Leu-NH)-2-propanone; peptidyl diazomethanes, Z-Phe-Ala-CHN2, Z-Phe-Thr(OBzl)-CHN2, Z-Phe-Tyr(Ot- But)-CHN2, Z-Leu-Leu-Tyr-CHN2; peptidyl acyloxymethyl ketones; peptidyl methylsulfonium salts; peptidyl vinyl sulfones, LHVS; peptidyl nitriles; peptidyl disulfides, 5,5′-dithiobis[2-nitrobenzoic acid], cysteamines, 2,2′-dipyridyl disulfide; non-covalent inhibitors, N-(4-Biphenyl acetyl)-S-methyl cysteine-(D)-Arg-Phe-b-phenethylamide; thiol alkylating agents, maleimides, etc; azapeptides; azobenzenes; O-acylhydroxamates, Z-Phe-Gly-NHO-Bz, Z-FG-NHO-BzOME; lysosomotropic agents, chloroquine, ammonium chloride; Cystatin A, Cystatin B, Cystatin C, Cystatin D, Cystatin F, stefins, kininogens, Sialostain L, antimicrobial peptide LL-37, Procathepsin B Fragment 26-50, Procathepsin B Fragment 36-50; Odanacatib (MK-0822), Balicatib (AAE581), Relacatib (GSK-462795, SB-462795), SLV213 (K777 or K1777), RO5459072, RWJ-445380, VBY036P1A, AM-3701, MIV-701, MIV-710, MIV-711, NC-2300, ORG-219517, ONO-5334, MK-0674, GB-111-NH2, L-873724, L-006235, AZD4996, VBY-036, RWY-445380, AM-3840, Cz-007, VBY-825 (VBY- 106; VBY-285;VBY-825), VBY-129, SAR-114137, VBY-891, Petesicatib (RG-7625; RO-5459072), LY-3000328, MIV32 247, CRA-028129, RG-7236, GSK2793660, Aloxistatin (E-64d, Loxistatin, EST), BI-1181181 (VTP-37948), VBY-376, Aloxistatin (Ab-007; E-64-d), Begacestat (GSI-953; WAY-210953), AL101 (BMS906024), BMS-986115 5 (AL-102), MK-0752 (L-000891675), EVP-0962 (EVP- 0015962), SAR-164653, KGP94, VEL-0230, and BLD2660.

In one embodiment, the cathepsin inhibitor can be Balicatib (AAE581).

Proteases are essential for virus replication. Many human pathogenic viruses use human enzymes to activate the viral proteins and successfully overtake the infected cell processes. For example, human cathepsins assist in the cleavage of viral proteins that are essential for the virus life cycle. These proteases can include but are not limited to cysteine proteases and proteinases, serine proteases, aspartic proteases. For example serine protease inhibitors like Camostat, Odalasvir, Femostat, or any other protease inhibitors currently in use for HIV such as atazanavir (Reyataz), darunavir (Prezista), fosamprenavir (Lexiva), indinavir (Crixivan), lopinavir/ritonavir (Kaletra), nelfinavir (Viracept), ritonavir (Norvir), saquinavir (Invirase), tipranavir (Aptivus), atazanavir/cobicistat (Evotaz), darunavir/cobicistat (Prezcobix); or other protease inhibitors use for other viruses such as HCV for example asunaprevir, boceprevir, grazoprevir, glecaprevir, paritaprevir, simeprevir, telaprevir, and in HBV treatment.

In some embodiments, a cathepsin inhibitor can be an agent whose main pharmacological effect is to inhibit the activity of the class of endosomal cysteine peptidases that can be enzymatically active in acidic pH or neutral pH. Examples of human cysteine proteases include but are not limited to cathepsins, which include but are not limited to cathepsin B, cathepsin L, cathepsin S, cathepsin-F, cathepsin-X, cathepsin K, cathepsin V, cathepsin W, cathepsin C, cathepsin O, and cathepsin H. Cathepsin inhibitors useful in non-human animals are often categorized differently but are known to those of skill in the art. Thus, the inhibitors include cathepsin inhibitors which are known to correspond with human cathepsin inhibitors. Inhibitors of these cathepsins, are useful according to methods of the disclosure. Many cathepsin inhibitors have been described in the literature and are well known and are commercially available.

### Dipeptide Dinitriles

In some embodiments, the compositions of the present disclosure can include dipeptide dinitriles. In one aspect, the mammalian protease inhibitor can be a dipeptide nitrile.

In some embodiments, the mammalian protease inhibitor has a structure of Formula (I)

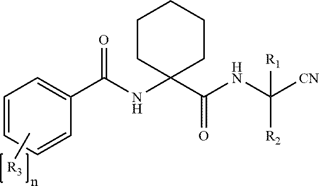

wherein, R1 and R2 are independently H or C1-C7 lower alkyl, or R1 and R2 together with the carbon atom to which they are attached form a C3-C8 cycloalkyl ring; and R3 is an optionally substituted heterocyclic group comprising at least one nitrogen; n is between 1 and 3.

In some embodiments, the mammalian protease inhibitor has a structure of Formula (II):

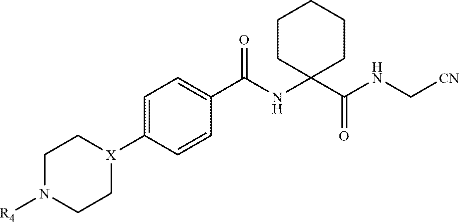

wherein X is CH or N; and R4 is H, C1-C7 lower alkyl, C1-C7 lower alkoxy, C5-C10 aryl, or C3-C8 cycloalkyl.

In some embodiments, the mammalian protease inhibitor has a structure of

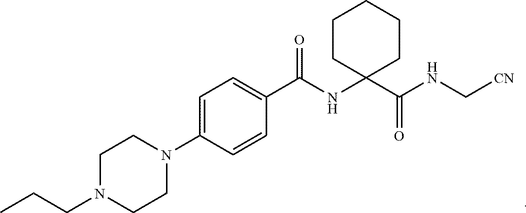

In some embodiments, the mammalian protease inhibitor has a structure of

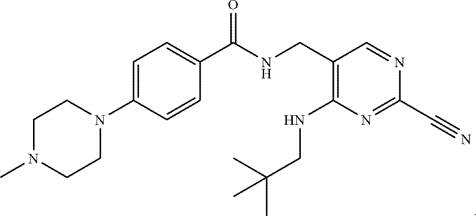

In some embodiments, the mammalian protease inhibitor has a structure of

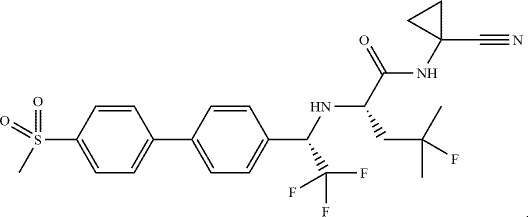

As a non-limiting example, the compositions of the disclosure can include a mammalian protease inhibitor of Formula (I) of the International Patent Publication WO2001058886 and provided below as Formula (III),

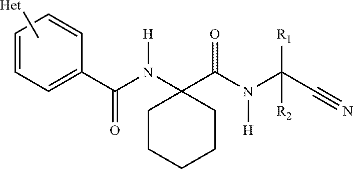

herein R1 and R2 are independently H or C1-C7 lower alkyl, or R1 and R2 together with the carbon atom to which they are attached form a C3-C8 cycloalkyl ring, and Het is an optionally substituted nitrogen-containing heterocyclic substituent.

In some embodiments, the compositions of the disclosure can include Formula (III), pharmaceutically acceptable salts or esters thereof.

In some embodiments, the mammalian protease inhibitor have a structure of Formula (IV)

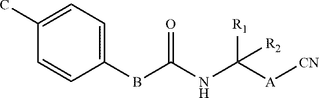

or a pharmaceutically acceptable salt thereof, wherein R1 and R2 are independently H, C1-C3 alkyl, C3-C6 cycloalkyl, or form a C3-C6 cycloalkyl group with the carbon to which they are attached, wherein the C1-C3 alkyl or the C3-C6 cycloalkyl is optionally substituted; A is a bond, C1-C3 alkyl, C6 aryl, or C6 heteroaryl, wherein the C1-C3 alkyl, C6 aryl, or C6 heteroaryl is optionally substituted; B is a bond, C1-C3 alkyl, an amide, an amine, wherein the C1-C3 alkyl, amide or amine is optionally substituted; and C is a C3-C8 cycloalkyl, C3-C8 heterocycloalkyl, C6 aryl, or C6 heteroaryl, wherein the C3-C8 cycloalkyl, C3-C8 heterocycloalkyl, C6 aryl, or C6 heteroaryl is optionally substituted.

In some embodiments, A is a C6 heteroaryl comprising one or two nitrogens. In some embodiments, A is optionally substituted

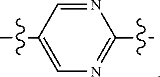

In some embodiments, B is optionally substituted —CH₂—NH—CH₂— or optionally substituted —CO—NH—CH₂—. [0096] In some embodiments, C is a substituted piperazine group

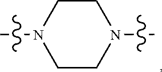

wherein the substituent can be a C1-C3 alkyl.

In some embodiments, C is a phenyl group

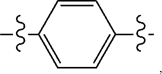

wherein the substituent can be a C1-C3 alkyl, halogen, or —SO₂—CH3.

In one aspect, the compositions of the disclosure can be or can include N-[1-(Cyanomethyl carbamoyl)cyclohexyl]4-[4-(1-propyl)piperalin-1-yl]benzamide, or a pharmaceutically acceptable salt. Non-limiting examples of the mammalian protease inhibitors useful in the present disclosure include, N-[1-(Cyanomethyl-carbamoyl)-cyclohexyl]-4-(piperazin-1-yl)-benzamide; N-[1-(Cyanomethyl-carbamoyl)-cyclohexyl]-4-(4-methyl-piperazin-1-yl)-benzamide; N-[r-(Cyanomethyl-carbamoyl)-cyclohexyl]-4-(4-ethyl-piperazin-1-yl)-benzamide; N-[1-(Cyanomethyl-carbamoyl)-cyclohexyl]-4-(4-isopropyl-piperazin-1-yl)-benzamide; N-[1-(Cyanomethyl-carbamoyl)-cyclohexyl]-4-(4~benzyl-piperazin-1-yl)-benzamide; N-[1-(Cyanomethyl-carbamoyl)-cyclohexyl]-4-[4-(2-methoxy-ethyl)-piperazin-1-yl]-benzamide; N-[1-(Cyanomethyl-carbamoyl)-cyclohexyl]-4-(-1-propyl-piperidin-4-yl)-benzamide; N-[1-(Cyanomethyl-carbamoyl)-cyclohexyl]-4-[1-(2-methoxy-ethyl)-piperidin-4-yl]-benzamide; N-[1-(Cyanomethyl-carbamoyl)-cyclohexyl]-4-(1-isopropyl-piperidin-4-yl)-benzamide; N-[1-(Cyanomethyl-carbamoyl)-cyclohexyl]-4-(1-cyclopentyl-piperidin-4-yl)-benzamide; N-[1-(Cyanomethyl-carbamoyl)-cyclohexyl]-4-(1-methyl-piperidin-4-yl)-benzamide, or N-[1-(Cyanomethyl-carbamoyl)-cyclohexyl]-4-(piperidin-4-yl)-benzamide; and/or or N-[1-(Cyanomethyl-carbamoyl)-cyclohexyl]-4-[4-(1-propyl)-piperazin-1-yl]-benzamide.

### Covalent Warheads

In some embodiments, the mammalian protease inhibitor of the present disclosure can comprise at least one warhead moiety. The warhead moiety can be any covalent binding modality that is capable of forming a covalent bond with a biological target. The warhead moiety can comprise one or more chemical groups, one or more of which is capable of forming a covalent bond with a biological target. In one embodiment, the warhead moiety comprises nitrile (-CN). As a non-limiting example, the mammalian protease inhibitors containing warhead moieties can comprise Formula I, Formula II, Formula III or Formula IV described herein.

### Antiviral Drugs

In some embodiments, the drug is an antiviral drug.

Antiviral drugs can be used for treating viral infections by inhibiting their entry into the cell, or inhibiting viral development by blocking viral RNA or DNA synthesis. Antiviral drugs of the present disclosure can be, for example, 5-substituted 2′-deoxyuridine analogues nucleoside analogues, pyrophosphate analogues, nucleoside reverse transcriptase inhibitors, nonnucleoside reverse transcriptase inhibitors, viral protease inhibitors, integrase inhibitors, entry inhibitors, acyclic guanosine analogues, acyclic nucleoside, and/or phosphonate analogues.

In some embodiments, the compositions of the disclosure can include antivirals drugs currently in use for the treatment of HIV such as, but not limited to, atazanavir (Reyataz), darunavir (Prezista), fosamprenavir (Lexiva), indinavir (Crixivan), lopinavir/ritonavir (Kaletra), nelfinavir (Viracept), ritonavir (Norvir), saquinavir (Invirase), tipranavir (Aptivus), atazanavir/cobicistat (Evotaz), darunavir/cobicistat (Prezcobix) or other protease inhibitors use for other viruses such as HCV for example Asunaprevir, Boceprevir, Grazoprevir, Glecaprevir, Paritaprevir, Simeprevir, and/or Telaprevir.Nucleotide analogs

In some embodiments, the compositions of the disclosure can include nucleotide analogs which are synthetic compounds which are similar to nucleotides, but which have an incomplete or abnormal deoxyribose or ribose group. Once the nucleotide analogs are in the cell, they are phosphorylated, producing the triphosphate formed which competes with normal nucleotides for incorporation into the viral DNA or RNA. Once the triphosphate form of the nucleotide analog is incorporated into the growing nucleic acid chain, it causes irreversible association with the viral polymerase and thus chain termination, thereby blocking viral replication by impairing DNA/RNA synthesis or by inhibiting cellular or viral enzymes involved in nucleoside/tide metabolism. A description of nucleotide analogs, their structure and function are found in the following references incorporated by reference thereto: Eyer et al., 2018, Nucleoside analogs as a rich source of antiviral agents active against arthropod-borne flaviviruses. Antiviral Chemistry and Chemotherapy 26, 1-28 (the contents of which are herein incorporated by reference in its entirety). Non-limiting examples of nucleotide analogs include, cidofovir, adefovir dipivoxil, and tenofovir disoproxil fumarate (tenofovir DF).

“Nucleoside analogs” In some embodiments, the compositions of the disclosure can include nucleoside analogs. The antiviral effects and their pharmacokinetic properties make some nucleoside analogs suitable for the treatment of acute infections caused by medically important RNA and DNA viruses. Many small molecule-based antivirals are nucleoside analogs, most commonly used for chemotherapy of chronic infections caused by HIV, hepatitis B or C viruses, and herpes viruses. There are currently over 25 approved therapeutic nucleosides used in therapy of various diseases ranging from viral infections to osteoporosis, to cancer. Non-limiting examples of nucleoside analogs include, BCX4430 (Galdisivir), T-705 (Favipiravir, Avigan), JNJ- 64041575, JNJ-1575, ALS-008176, AL-8176 (Lumicitabine), Brincidofovir, Camostat, Odalasvir, FGE-106, JK-05, Nafamostat mesylate, Triazavirin, Acyclovir Fleximer and other Fleximers, Ribavirin, AL-335 (Adafosbuvir), 6-azauridine, Acyclovir, gancyclovir, idoxuridine, dideoxyinosine, dideoxycytidine, zidovudine (azidothymidine), imiquimod, resimiquimod, remdesivir, gemcitabine hydrocholoride, mizoribine, lamivudine, entecavir, telbivudine, adefovir dipivoxil, tenofovir disoproxil fumarate (TDF), sofosbuvir, FV100 (FV for FermaVir), letermovir, daclatasvir, asunaprevir, beclabuvir, lopinavir, ritonavir, Hepsera (adefovir dipivoxil), Peveon, Viread (tenofovir disoproxil fumarate), Acycloadenosine (predecessor of acyclovir), NITD008, MK-608, ribonucleoside analog β-d-N4-hydroxycytidine (NHC; EIDD-1931), EIDD-2801 (Molnupiravir), AT-527, and AT-511. As the methods and compositions of the disclosure provide synergistic activity in these drugs, these analogs can potentially be repurposed for treatment of other infections and diseases.

In some embodiments, the nucleoside analog can be Trifluridine, Brivudine, Vidarabine (Vidarabine Phosphate, Vidarabine Sodium Phosphate), Tellbivudine, Didanosine, Zalcitabine, Stavudine, Lamivudine, Abacavir, Emtricitabine, Sorivudine, Fialuridine, Fiacitabine.

“Additional antiviral drugs” Other anti-viral drugs can be combined with the protease inhibitors e.g., cathepsin inhibitors of the disclosure. Examples of additional antiviral drugs include but are not limited to immunoglobulins, amantadine, interferons. Examples of anti-viral agents include, but are not limited to, Idoxuridine, Trifluridine Brivudine, Vidarabine, Entecavir, Tellbivudine, Foscarnet, Zidovudine, Didanosine, Zalcitabine, Stavudine, Lamivudine, Lamivudine in combination with zidovudine, Abacavir, Abacavir in combination with lamivudine and zidovudine, Emtricitabine, Nevirapine, Delavirdine, Efavirenz, Etravirine, Etravirine, Rilpivirine, Saquinavir, Ritonavir, Indinavir, Nelfinavir, Amprenavir, Lopinavir- ritonavir, Atazanavir, Tipranavir, Darunavir, Darunavir in combination with cobicistat, Atazanavir in combination with cobicistat, Telaprevir, Boceprevir, Simeprevir, Asunaprevir, Vaniprevir in combination with ribavirin and PegIFNox-2b, Paritaprevir, Paritaprevir, Grazoprevir, Raltegravir, Elvitgravir, Dolutegravir, Dolutegravir in combination with abacavir and lamivudine, Dolutegravir in combination with lamivudine, RSV-IGIV, Palvizumab, Docosanol, Enfuvirtide, Maraviroc, VZIG, VariZIG, Acyclovir, Ganciclovir, Famciclovir, Valacyclovir, Penciclovir, Valganciclovir, Cidofovir, Tenofovir disoproxil fumarate, Adefovir dipivoxil, Tenofovir disoproxil fumarate in combination with emtricitabine, Tenofovir disoproxil fumarate in combination with efavirenz and emtricitabine, Tenofovir disoproxil fumarate in combination with rilpivirine and emtricitabine, Tenofovir disoproxil fumarate in combination with cobicistat and Elvitegravir, Tenofovir alafenamide in combination with cobicistat, emtricitabine and elvitegravir, Tenofovir alafenamide in combination with rilpivirine and emtricitabine, Tenofovir alafenamide in combination with emtricitabine, Sofosbuvir in combination with ribavirin, Sofosbuvir in combination with ribavirin and PegIFNox, Daclatasvir in combination with asunaprevir, Ledipasvir in combination with sofosbuvir, Sofosbuvir in combination with simeprevir, Ombitasvir in combination with dasabuvir, paritaprevir and ritonavir, Ombitasvir in combination with paritaprevir and ritonavir, Daclatasvir in combination with sofosbuvir, Elbasvir in combination with grazoprevir, Amantadine, Ribavirin, AL-335 30 (Adafosbuvir), Rimantadine, Rimantadine, Zanamivir, Olseltamivir, Laninamivir octanoate, Laninamivir octanoate, Peramivir, Favipiravir, Pegylated interferon alfa 2b, Interferon alfacon , Pegylated interferon alfa 2b in combination with ribavirin, Pegylated interferon alfa 2a, Fomivirsen, Podofilox, Imiquimod, Sinecatechins, Acemanan, Acyclovir Sodium, Alovudine, Alvircept Sudotox, Amantadine Hydrochloride, Alvircept Sudotox, Amantadine Hydrochloride, Aranotin, Arildone, Aegirine Mesylate, Avridine, Cidofovir, Cipamfylline, Cytarabine Hydrochloride, Delavirdine Mesylate, Desciclovir, Didanosine, Disoxaril Edoxudine, Enviradene, Enviroxime, Famciclovir, Famotine Hydrochloride, Fiacitabine, Fialuridine, Fosarilate, Foscarnet Sodium, Fosfonet Sodium, Ganciclovir, Ganciclovir Sodium, Idoxuridine, Kethoxal, Lamivudine, Lobucavir, Memotine Hydrochloride, Methisazone, Nevirapine, Penciclovir, Pirodavir, Ribavirin, AL-335 (Adafosbuvir), Rimantadine Hydrochloride, Saquinavir Mesylate, Somantadine Hydrochloride, Sorivudine, Statolon, Stavudine, Tilorone Hydrochloride, Trifluridine, Valacyclovir Hydrochloride, Vidarabine, Vidarabine Phosphate , Vidarabine Sodium Phosphate, Viroxime, Zalcitabine , Zidovudine, and/or Zinviroxime.

“Viral protease inhibitors” In some embodiments, the antiviral drug is a viral protease inhibitor. As used herein a protease inhibitor is a molecule that blocks or reduces the activity of a protease. Proteases are essential for virus replication. Specifically, a viral protease inhibitor refers to a protease inhibitor that is capable of targeting and reducing the activity of a viral protease.

In some embodiments, the viral protease inhibitor can be a viral serine protease inhibitor, such as, but not limited to, Camostat, Odalasvir, Femostat, or any other protease inhibitors currently in use for HIV such as atazanavir (Reyataz), darunavir (Prezista), fosamprenavir (Lexiva), indinavir (Crixivan), lopinavir/ritonavir (Kaletra), nelfinavir (Viracept), ritonavir (Norvir), saquinavir (Invirase), tipranavir (Aptivus), atazanavir/cobicistat (Evotaz), darunavir/cobicistat (Prezcobix) or other protease inhibitors use for other viruses such as HCV for example asunaprevir, boceprevir, grazoprevir, glecaprevir, paritaprevir, simeprevir, telaprevir, and in HBV treatment, can be useful in the present disclosure.

In some embodiments, the protease inhibitor can a viral protease inhibitor. Viral proteases such as SARS-CoV proteases are essential for viral life-cycle and these proteases can be inhibited by compounds such as, but not limited to, PF-07321332, PF-07304814, and PF-00835231.

In some embodiments, the viral protease inhibitor can be Indinavir, Nelfinavir, Saquinavir (Saquinavir Mesylate), Ritonavir, Amprenavir, Lopinavir, ritonavir, Atazanavir, Tipranavir, Darunavir, Telaprevir, Boceprevir, Simeprevir, Asunaprevir, Vaniprevir in combination with ribavirin, Paritaprevir, Paritaprevir, Grazoprevir, Ganciclovir (Ganciclovir Sodium), Famciclovir, Valacyclovir (Valacyclovir Hydrochloride), Penciclovir, and/or Valganciclovir.

“Viral polymerase inhibitors” In some embodiments, the antiviral drug can be a polymerase inhibitor. Non-limiting examples of viral polymerase inhibitors include Foscarnet (Foscarnet Sodium, Fosfonet Sodium), Cidofovir, and/or Alovudine. “Reverse transcriptase inhibitors” In some embodiments, the antiviral drug can be a reverse transcriptase inhibitor. Non limiting examples of reverse transcriptase enzyme inhibitors include, Nevirapine, Delavirdine (Delavirdine Mesylate), Efavirenz, Etravirine, Rilpivirine, Adefovir dipivoxil, and/or Atevirdine.

“Viral integrase inhibitors” In some embodiments, the antiviral drug can be a viral integrase inhibitor. Non-limiting examples of integrase inhibitor include, Raltegravir, Elvitegravir, and/or Dolutegravir.

“Viral envelope fusion inhibitors” In some embodiments, the antiviral drug can be an agent that inhibits the fusion of the viral envelope with the host cell membrane. Non-limiting examples of viral envelope fusion inhibitors include Docosanol, Enfuvirtide, and/or Maraviroc.

“Prophylactic agents” In some embodiments, the antiviral drug can be a prophylactic agent such as a vaccine. Non-limiting examples of vaccines include, RSV-IGIV, VZIG, and/or VariZIG.

“Protein drugs” In some embodiments, the antiviral drug can be a protein drug. As used herein, a protein drug refers to a drug having protein characteristics including large molecular weight. In some embodiments, the drug can be an antibody. Non-limiting examples of antibodies include Palvizumab, Atoltivimab, maftivimab, odesivimab, and/or Zmapp. In some embodiments, the protein drug can be pegylated interferon alfa 2b, interferon alfacon, and/or a pegylated interferon alfa 2a.

“Proton transport inhibitors” In some embodiments, the antiviral drug is a proton transport inhibitor. Non-limiting examples of proton transport inhibitors include Rimantadine (Rimantadine Hydrochloride), and/or Methisazone.

Neuraminidase inhibitors” In some embodiments, the antiviral drug is a neuraminidase inhibitor. Non-limiting examples of neuraminidase inhibitors include, Zanamivir, Oseltamivir, Laninamivir octanoate, Laninamivir octanoate, and/or Peramivir.

“Antiviral drug combinations” In some embodiments, the compositions of the disclosure can include two or more antiviral drugs. Non-limiting examples of antiviral drugs combinations include, Tenofovir disoproxil fumarate, Tenofovir disoproxil fumarate in combination with emtricitabine , Tenofovir disoproxil fumarate in combination with efavirenz and emtricitabine, Tenofovir disoproxil fumarate in combination with rilpivirine and emtricitabine, Tenofovir disoproxil fumarate in combination with cobicistat and Elvitegravir, Tenofovir alafenamide in combination with cobicistat, emtricitabine and elvitegravir, Tenofovir alafenamide in combination with rilpivirine and emtricitabine, Tenofovir alafenamide in combination with emtricitabine, Sofosbuvir in combination with ribavirin, Sofosbuvir in combination with ribavirin and PegIFNox , Daclatasvir in combination with asunaprevir, Ledipasvir in combination with sofosbuvir, Sofosbuvir in combination with simeprevir, Ombitasvir in combination with dasabuvir, paritaprevir and ritonavir, Ombitasvir in combination with paritaprevir and ritonavir, Daclatasvir in combination with sofosbuvir, Elbasvir in combination with grazoprevir, Dolutegravir in combination with abacavir and lamivudine, Dolutegravir in combination with lamivudine, Pegylated interferon alfa 2b in combination with ribavirin, Abacavir in combination with lamivudine and zidovudine, Darunavir in combination with cobicistat, Atazanavir in combination with cobicistat.

“Other antiviral drugs” In some embodiments, antiviral drugs of the disclosure can be Amantadine, AL-335, Podofilox, Imiquimod, Sinecatechins, Acemanan, Statolon, Somantadine Hydrochloride, Pirodavir, Memotine Hydrochloride, Lobucavir, Kethoxal, Fosarilate, Famotine, Enviroxime, Enviradene, Disoxaril Edoxudine, Desciclovir, Cytarabine Hydrochloride, Cipamfylline, Avridine, Arildone, Tilorone Hydrochloride, Viroxime, Zalcitabine, Fomivirsen and/or Zinviroxime.

### Combinations

During the drug development pathway, a vast number of candidate drugs are abandoned because of observed serious adverse reactions. In many cases, these adverse side effects are a result of the large doses required of the intrinsically toxic drug for therapeutic effect when the efficacy of the drug is low. An effective strategy for eliminating the toxicity of these drugs is to lower the required dosage by increasing the drug’s efficacy. In order to prevent or reduce the effects of a pandemic, a need also exists to further develop countermeasures against new and virulent strains as a monotherapy or in combination with other anti-virals. Therefore, there is a need for a method for increasing the efficacy of a drug and reducing the required effective dose. In some embodiments, the compositions and methods described herein meet this need.

The present disclosure provides methods wherein a composition provides an increase in bioavailability of a drug, such as an anti-viral drug, when combined with a protease inhibitor, for example a cathepsin inhibitor, as measured by AUC (Area Under the Curve) of at least 25% relative to dosing of the drug alone. The present disclosure also provides methods wherein the composition provides an increase in bioavailability of the drug combination as measured by AUC of at least 50% relative to dosing of the drug alone. The present disclosure further provides methods wherein said composition provides an increase in bioavailability of the drug in combination as measured by AUC of at least 100% relative to dosing of the drug alone.

Certain small molecules have unexpected anti-viral activity, as they were developed and examined for non-viral disease states. These small molecules act as an anti-viral whether as a monotherapy or in combination with other anti-viral drugs. The small molecules can demonstrate anti-viral activity across all virus classes, in which anti-viral activity has been proven. The efficacy of these small molecules is especially high against Coronaviruses (i.e., SARS-CoV-2.)

These small molecules can be effective as a pre-exposure prophylaxis during a viral outbreak, for healthcare workers, first responders, at-risk workers, or travelers. These small molecules can be effective as a post-exposure prophylaxis as well. The post-exposure treatment can be used to prevent a fatal or severe infection and in a “track, trace, and treat” program implementation. Even with a viable vaccine, this form of treatment is necessary. These small molecules can treat mild and severe viral infections. As a treatment for infections, these small molecules can treat the viral load and prevent or reduce hospital stays and reduce the burden on the healthcare system. This treatment can be effective when dealing with multiple non-viral complications including organ failure, ARDS, pneumonia, or other side effects.

These small molecules can be effective in a wide array of patient populations include those over 65, patients with comorbidities, and immunosuppressed patients.

The disclosure provides methods for the identification of a mammalian protease inhibitor that produces synergistic activity with a drug of choice. In certain aspects, the disclosure provides methods for the identification of a mammalian protease inhibitor that reduces the effective dosage of a drug of choice. Any technique known to the skilled artisan can be used to screen for a mammalian protease inhibitor that would reduce the effective dose of a drug. As an example, a cell is contacted with a test protease inhibitor in combination with a drug of choice, for example an antiviral drug. A control without the test protease inhibitor is provided. The cell can be contacted with a test protease inhibitor before, concurrently with, or subsequent to the administration of the drug. A cell can be incubated with multiple concentrations of a drug and test protease inhibitor, for at least 1 minute to at least 10 minutes during the experiment. The effect of the combination on the viral replication can be measured at various times during the assay. A time course of viral replication in the culture was determined. If the viral replication is inhibited or reduced in the presence of the mammalian protease inhibitor at reduced drug concentrations wherein the effect is more than an additive effect, the test protease inhibitor is identified as being effective in producing a synergistic activity.

The combinations of antiviral drugs and mammalian protease inhibitors of the disclosure can be combined with other therapeutic agents. Drug combinations of the disclosure can include, but are not limited to, for example, Relacatib (GSK-462795, SB-462795) in combination with PF-07321332, PF-07304814, or PF-00835231 for use in treatment of pathogenic CoV such as SARS-CoV. Relacatib (GSK-462795, SB-462795) in combination with AT-527 or AT-511 for use in treatment of pathogenic CoVs such as SARS-CoVs. SLV-213, K777, or K-1777 in combination with AT-527 or AT-511 for use in pathogenic CoVs such as SARS CoVs. SLV-213, K777, or K-1777 in combination with PF-07321332, PF-07304814, or PF-00835231 for use in treatment of pathogenic CoVs such as SARS-CoVs. SLV-213, K777, or K-1777 in combination with T-705, GS-5734 (remdesivir) for treatment of pathogenic CoVs such as SARS-CoVs. Relacatib (GSK-462795, SB-462795), in combination with T-705 (Avigan) for use in the treatment of arenavirus infections; Relacatib (GSK-462795, SB-462795), in combination with T-705 (Avigan) for use in the treatment of SARS-CoV-2 infections; MIV-711, in combination with T-705 (Favipiravir, Avigan) for use in treatment of SARS CoV-2 and other coronaviruses such as MERS and SARSCoV; AM-3701, MIV-701, MIV-710, MIV-711, NC-2300, ORG-219517 or Relacatib (GSK-462795, SB-462795), in combination with JNJ-64041575, JNJ-1575, ALS-008176, AL-8176 (Lumicitabine) for use in the treatment of RSV infections; AM-3701, MIV-701, MIV-710, MIV-711, NC-2300, ORG-219517 or Relacatib (GSK-462795, SB-462795), in combination with JNJ-64041575, JNJ-1575, ALS-008176, AL-8176 (Lumicitabine) for use in the treatment of SARS-CoV-2 infections; AM-3701, MIV-701, MIV-710, MIV-711, NC-2300, ORG-219517 or Relacatib (GSK-462795, SB-462795) or Odanacatib (MK-0822) or Balicatib (AAE581), in combination with JNJ-64041575, JNJ-1575, ALS-008176, AL-8176 (Lumicitabine) for use in the treatment of RSV infections; AM-3701, MIV-701, MIV-710, MIV-711, NC-2300, ORG-219517 or Relacatib (GSK-462795, SB-462795) in combination with T-705 (Avigan, Favipiravir) or BCX 4430 (Galidesivir) for use in the treatment of SARSCoV-2 infections; AM-3701, MIV-701, MIV-710, MIV-711, NC-2300, ORG-219517 or Relacatib (GSK-462795, SB-462795), in combination with GS-5734 (Redmdesivir) in the treatment of SARS-CoV-2 infections; Odanacatib (MK-0822) or Balicatib (AAE581), in combination with GS-5734 (Redmdesivir) in the treatment of SARS-CoV-2 infections; Relacatib (GSK-462795, SB-462795), in combination with T-705 (Avigan, Favipiravir) along with lopinavir/ritonavir (Kaletra) for use in the treatment of filovirus infections; Relacatib (GSK-462795, SB-462795), in combination with T-705 (Avigan), along with lopinavir/ritonavir (Kaletra) for use in the treatment of coronavirus infections such as SARSCoV-2 infections; Relacatib (GSK-462795, SB-462795), in combination with T-705 (Avigan), along with lopinavir/ritonavir (Kaletra) for use in the treatment of coronavirus infections such as SARS- CoV-2 and other coronaviruses such as MERS and SARSCoV; AM-3701, MIV-701, MIV-710, MIV-711, NC-2300, ORG-219517 or Relacatib (GSK-462795, SB-462795), in combination with JNJ-64041575, JNJ-1575, ALS-008176, AL-8176 (Lumicitabine) for use in the treatment of RSV infections; AM-3701, MIV-701, MIV-710, MIV-711, NC-2300, ORG-219517 or Relacatib (GSK-462795, SB-462795), in combination with BCX4430 (Galidesivir), T-705 (Avigan, Favipiravir) along with a mammalian protease inhibitor such as darunavir (Prezista) in the treatment of coronavirus infections such as SARSCoV- 2 infections; AM-3701, MIV-701, MIV-710, MIV-711, NC-2300, ORG-219517 or Relacatib (GSK-462795, SB-462795), in combination with GS-5734 (Remdesivir), along with a mammalian protease inhibitor such as darunavir (Prezista) in the treatment of coronavirus infections such as SARS-CoV-2 infections; Odanacatib (MK-0822) or Balicatib (AAE581), in combination with GS-5734 (Remdesivir), along with a mammalian protease inhibitor such as darunavir (Prezista) in the treatment of coronavirus infections such as SARS-CoV-2 and other pathogenic CoVs infections. Antiviral antibodies in combination with direct antiviral such as AT-527 and/or cathepsin inhibitors such as Balicatib, SLV-213.

In some embodiments, the combination or mixture of the present disclosure or a formulation thereof is administered in conjunction with the other therapeutic modality. In certain such embodiments, the other therapeutic modality is one that is normally administered to patients with the disease to be treated or prevented.

### In Vitro Assays for Testing Combinations

The combinations of the disclosures can be tested for in vitro activity against a disease or microorganism and sensitivity, and for cytotoxicity in laboratory adapted cell lines or cultured cells such as peripheral blood mononuclear cells (PBMC), human fibroblast cells, hepatic, renal, epithelium cells, according to standard assays developed for testing compounds. Combination assays can be performed at varying concentrations of the antiviral drug and/or the mammalian protease inhibitor to determine EC₅₀ by serial dilutions.

Cells: HEp-2 (CCL-23), PC-3 (CCL-1435), HeLa (CCL-2), U2OS (HTB-96), Vero (CCL-81), HFF-1 (SCRC-1041), and HepG2 (HB-8065) cell lines can be purchased from the American Type Culture Collection. HEp-2 cells can be cultured in Eagle’s Minimum Essential Media (MEM) with GlutaMAX supplemented with 10% fetal bovine serum (FBS) and 100 U ml-1 penicillin and streptomycin. PC-3 cells can be cultured in Kaighn’s F12 media supplemented with 10% FBS and 100 U ml-1 penicillin and streptomycin. HeLa, U2OS, and Vero cells can be cultured in MEM supplemented with 10% FBS, 1% L-glutamine, 10 mM HEPES, 1% non-essential amino acids, and 1% penicillin/streptomycin. HFF-1 cells can be cultured in MEM supplemented with 10% FBS and 0.5 mM sodium pyruvate. HepG2 cells can be cultured in Dulbecco’s Modified Eagle Medium (DMEM) with GlutaMAX supplemented with 10% FBS, 100 U ml-1 penicillin and streptomycin, and 0.1 mM non-essential amino acids. The MT-4 cell line can be obtained from the NIH AIDS Research and Reference Reagent Program and cultured in RPMI-1640 medium supplemented with 10% FBS, 100 U ml-1 penicillin and streptomycin, and 2 mM L-glutamine. The Huh-7 cell line can be obtained from C. M. Rice (Rockefeller University) and cultured in DMEM supplemented with 10% FBS, 100 U ml-1 penicillin and streptomycin, and non-essential amino acids.

Primary human hepatocytes or other primary cell can be purchased from Invitrogen and cultured in William’s Medium E medium containing cell maintenance supplement. Donor profiles will be limited to 18- to 65-year-old non-smokers with limited alcohol consumption. Upon delivery, the cells will be allowed to recover for 24 h in complete medium with supplement provided by the vendor at 37° C. Human PBMCs will be isolated from human buffy coats obtained from healthy volunteers (Stanford Medical School Blood Center, Palo Alto, California) and maintained in RPMI-1640 with GlutaMAX supplemented with 10% FBS, 100 U ml-1 penicillin and streptomycin.

To test viral inhibition in primary nonhuman primate cells, Rhesus fresh whole blood will be obtained from Valley Biosystems or other suppliers. PBMCs will be isolated from whole blood by Ficoll-Hypaque density gradient centrifugation. Briefly, blood will be overlaid on 15 ml Ficoll-Paque (GE Healthcare Bio-Sciences AB), and centrifuged at 500 g for 20 min. The top layer containing platelets and plasma will be removed, and the middle layer containing PBMCs will be transferred to a fresh tube, diluted with Tris buffered saline up to 50 ml, and centrifuged at 500 g for 5 min. The supernatant will be removed and the cell pellet will be resuspended in 5 ml red blood cell lysis buffer (155 mM ammonium chloride, 10 mM potassium bicarbonate, 0.1 mM EDTA, pH 7.5). To generate stimulated PBMCs, freshly isolated quiescent PBMCs will be seeded into a T-150 (150 cm2) tissue culture flask containing fresh medium supplemented with 10 U ml-1 of recombinant human interleukin-2 (IL-2) and 1 µg ml-1 phytohaemagglutinin-P at a density of 2 × 106 cells ml-1 and incubated for 72 h at 37° C. Human macrophage cultures will be isolated from PBMCs that will be purified by Ficoll gradient centrifugation from 50 ml of blood from healthy human volunteers. PBMCs will be cultured for 7 to 8 days in in RPMI cell culture media supplemented with 10% FBS, 5 to 50 ng ml-1 granulocyte-macrophage colony-stimulating factor and 50 µM β-mercaptoethanol to induce macrophage differentiation. The cryopreserved human primary renal proximal tubule epithelial cells will be obtained from LifeLine Cell Technology and isolated from the tissue of human kidney. The cells will be cultured at 90% confluency with RenaLife complete medium in a T-75 flask for 3 to 4 days before seeding into 96-well assay plates. Immortalized human microvascular endothelial cells (HMVEC-TERT) will be obtained from R. Shao at the Pioneer Valley Life Sciences Institute. HMVEC-TERT cells will be cultured in endothelial basal media supplemented with 10% FBS, 5 µg of epithelial growth factor, 0.5 mg hydrocortisone, and gentamycin/amphotericin-B. The percentage of viral inhibition can be 10-20%, 15-25%, 20-30%, 25-35%, 30-40%, 35-45%, 40-50%, 45-55%, 50-60%, 55-65%, 60-70%, 65-75%, 70-80%, 75-85%, 80-90%, 85-95%, 90-100%, or 95-100%.

In some experiments the intracellular metabolism (phosphorylation) of nucleobase and nucleoside (Nuc) can be evaluated. These studies can be performed as below.

In some embodiments, the intracellular metabolism of nucleoside can be assessed in different cell types (HMVEC and HeLa cell lines, and primary human and rhesus PBMCs, monocytes and monocyte-derived macrophages) following 2-h pulse or 72-h continuous incubations with 10-1,000 µM of nucleobase or nucleoside. For comparison, intracellular metabolism during a 72-h incubation with 10-1,000 µM of Nuc will be completed in human monocyte-derived macrophages. For pulse incubations, monocyte-derived macrophages isolated from rhesus monkeys or humans will be incubated for 2 h in media containing antiviral drug and/or the mammalian protease inhibitor followed by removal, Uimethylhexylamine (DMH) in water for analysis by liquid chromatography coupled to triple quadrupole mass spectrometry (LC-MS/MS).

In some embodiments, LC-MS/MS can be performed using low-flow ion-pairing chromatography, similar to methods described previously (Durand-Gasselin L, et al. Nucleotide analogue prodrug tenofovir disoproxil enhances lymphoid cell loading following oral administration in monkeys. Mol. Pharm. 2009; 6:1145-1151). Analytes can be separated using a 50 × 2 mm × 2.5 µm Luna C18(2) HST column (Phenomenex) connected to a LC-20ADXR (Shimadzu) ternary pump system and HTS PAL autosampler (LEAP Technologies). A multi-stage linear gradient from 10% to 50% acetonitrile in a mobile phase containing 3 mM ammonium formate (pH 5.0) with 10 mM dimethylhexylamine over 8 min at a flow rate of 150 µl min-1 can be used to separate analytes. Detection can be performed on an API 4000 (Applied Biosystems) MS/MS operating in positive ion and multiple reaction monitoring modes. Intracellular metabolites alanine metabolite, Nuc, nucleoside monophosphate, nucleoside diphosphate, and nucleoside triphosphate can be quantified using 7-point standard curves ranging from 0.274 to 200 pmol (approximately 0.5 to 400 µM) prepared in cell extract from untreated cells. Levels of adenosine nucleotides can be also quantified to assure dephosphorylation had not taken place during sample collection and preparation. In order to calculate intracellular concentration of metabolites, the total number of cells per sample can be counted using a Countess automated cell counter (Invitrogen).

In some embodiments, Ebola Antiviral testing can be conducted in a biosafety level 4 containment (BSL-4), for example at the Centers for Disease Control and Prevention. EBOV antiviral assays can be conducted in primary HMVEC-TERT and in Huh-7 cells. Huh-7 cells will not be authenticated and will not be tested for mycoplasma. Ten concentrations of antiviral drugs and/or mammalian protease inhibitors can be diluted in fourfold serial dilution increments in media, and 100 µl per well of each dilution can be transferred in duplicate (Huh-7) or quadruplicate (HMVEC-TERT) onto 96-well assay plates containing cell monolayers. The plates can be transferred to BSL-4 containment, and the appropriate dilution of virus stock can be added to test plates containing cells and serially diluted antiviral drugs and/or mammalian protease inhibitors. Each plate will include four wells of infected untreated cells and four wells of uninfected cells that serve as 0% and 100% virus inhibition controls, respectively. After the infection, assay plates can be incubated for 3 days (Huh-7) or 5 days (HMVEC-TERT) in a tissue culture incubator. Virus replication can be measured by direct fluorescence using a Biotek HTSynergy plate reader. For virus yield assays, Huh-7 cells can be infected with wild-type Ebola Virus (also herein EBOV) for 1 h at 0.1 plaque-forming units (PFU) per cell. The virus inoculum can be removed and replaced with 100 µl per well of media containing the appropriate dilution of antiviral drugs and/or mammalian protease inhibitors. At 3 days post-infection, supernatants can be collected, and the amount of virus can be quantified by endpoint dilution assay. The endpoint dilution assay can be conducted by preparing serial dilutions of the assay media and adding these dilutions to fresh Vero cell monolayers in 96-well plates to determine the tissue culture infectious dose that caused 50% cytopathic effects (TCID₅₀). To measure levels of viral RNA from infected cells, total RNA can be extracted using the MagMAX-96 Total RNA Isolation Kit and quantified using a quantitative reverse transcription polymerase chain reaction (qRT-PCR) assay with primers and probes specific for the EBOV nucleoprotein gene.

In some embodiments, antiviral assays can be conducted in BSL-4. HeLa or HFF-1 cells can be seeded at 2,000 cells per well in 384-well plates. Ten serial dilutions of antiviral drugs and/or mammalian protease inhibitors in triplicate can be added directly to the cell cultures using the HP D300 digital dispenser (Hewlett Packard) in twofold dilution increments starting at 10 µM at 2 h before infection. The DMSO concentration in each well can be normalized to 1% using an HP D300 digital dispenser. The assay plates can be transferred to the BSL-4 suite and infected with EBOV Kikwit at a multiplicity of infection of 0.5 PFU per cell for HeLa cells and with EBOV Makona at a multiplicity of infection of 5 PFU per cell for HFF-1 cells. The assay plates can be incubated in a tissue culture incubator for 48 h. Infection can be terminated by fixing the samples in 10% formalin solution for an additional 48 h before immune-staining, as described. A decrease in the MOI is expected upon treatment with antiviral drugs and/or mammalian protease inhibitors. The percentage decrease in MOI with Ebola relative to untreated cells can be about 5%, 10%, 15%, 20%, 25%, 30%, 35%, 40%, 45%, 50%, 55%, 60%, 65%, 70%, 75%, 80%, 85%, 90%, 95% or 100% or between about 2%-5%, about 5%-10%, about 10%-20%, about 20%-30%, about 30%-40%, about 40%-50%, about 50%-60%, about 60%-70%, about 70%-80%, about 80%-90%, about 90%-100%.

In some embodiments, antiviral assays in EBOV human macrophages can be conducted in BSL-4. Primary human macrophage cells can be seeded in a 96-well plate at 40,000 cells per well. Eight to ten serial dilutions of antiviral drugs and/or mammalian protease inhibitors in triplicate can be added directly to the cell cultures using an HP D300 digital dispenser in threefold dilution increments 2 h before infection. The concentration of DMSO can be normalized to 1% in all wells. The plates can be transferred into the BSL-4 suite, and the cells can be infected with 1 PFU per cell of EBOV in 100 µl of media and incubated for 1 h. The inoculum can be removed, and the media can be replaced with fresh media containing diluted antiviral drugs and/or mammalian protease inhibitors. At 48 h post-infection, virus replication can be quantified by immuno-staining. Treatment with antiviral drugs and/or mammalian protease inhibitors is expected to decrease viral replication by about 5%, 10%, 15%, 20%, 25%, 30%, 35%, 40%, 45%, 50%, 55%, 60%, 65%, 70%, 75%, 80%, 85%, 90%, 95% or 100% or between about 2%-5%, about 5%-10%, about 10%-20%, about 20%-30%, about 30%-40%, about 40%-50%, about 50%-60%, about 60%-70%, about 70%-80%, about 80%-90%, about 90%-100%.

For RSV A2 antiviral tests, antiviral drugs and/or mammalian protease inhibitors can be threefold serially diluted in source plates from which 100 ml of diluted antiviral drugs and/or mammalian protease inhibitors can be transferred to a 384-well cell culture plate using an Echo acoustic transfer apparatus. HEp-2 cells can be added at a density of 5 × 10⁵ cells per ml, then infected by adding RSV A2 at a titer of 1 × 10⁴ tissue culture infectious doses (TCID₅₀) per ml. Immediately following virus addition, 20 µl of the virus and cells mixture can be added to the 384-well cell culture plates using a µFlow liquid dispenser and cultured for 4 days at 37° C. After incubation, the cells can be allowed to equilibrate to 25° C. for 30 min. The RSV-induced cytopathic effect can be determined by adding 20 µl of CellTiter-Glo Viability Reagent. After a 10-min incubation at 25° C., cell viability can be determined by measuring luminescence using an Envision plate reader. Cell viability is expected to be increased by about 5%, 10%, 15%, 20%, 25%, 30%, 35%, 40%, 45%, 50%, 55%, 60%, 65%, 70%, 75%, 80%, 85%, 90%, 95% or 100% or between about 2%-5%, about 5%-10%, about 10%-20%, about 20%-30%, about 30%-40%, about 40%-50%, about 50%-60%, about 60%-70%, about 70%-80%, about 80%-90%, about 90%-100% when the cells are treated with antiviral drugs and/or mammalian protease inhibitors.

In some embodiments, antiviral assays can be conducted in 384-or 96-well plates in BSL-4 using a high-content imaging system to quantify virus antigen production as a measure of virus infection. A ‘no virus’ control and a ‘1% DMSO’ control can be included to determine the 0% and 100% virus infection, respectively. The primary and secondary antibodies and dyes used for nuclear and cytoplasmic staining are listed. The primary antibody specific for a particular viral protein can be diluted 1,000-fold in blocking buffer (1 × PBS with 3% BSA) and added to each well of the assay plate. The assay plates can be incubated for 60 min at room temperature. The primary antibody can be removed, and the cells can be washed three times with 1 × PBS. The secondary detection antibody can be an anti-mouse (or rabbit) IgG conjugated with Dylight488 (Thermo Fisher Scientific, catalogue number 405310). The secondary antibody can be diluted 1,000-fold in blocking buffer and can be added to each well in the assay plate. Assay plates can be incubated for 60 min at room temperature. Nuclei can be stained using Draq5 (Biostatus) or 33342 Hoechst (ThermoFisher Scientific) for Vero and HFF-1 cell lines. Both dyes can be diluted in 1 × PBS. The cytoplasm of HFF-1 (EBOV assay) and Vero E6 (MERS assay) cells can be counter-stained with CellMask Deep Red (Thermo Fisher Scientific). Cell images can be acquired using a Perkin Elmer Opera confocal plate reader (Perkin Elmer) using a ×10 air objective to collect five images per well. Virus-specific antigen can be quantified by measuring fluorescence emission at a 488 nm wavelength and the stained nuclei can be quantified by measuring fluorescence emission at a 640 nm wavelength. Acquired images can be analyzed using Harmony and Acapella PE software. The Draq5 signal can be used to generate a nuclei mask to define each nucleus in the image for quantification of cell number. The CellMask Deep Red dye can be used to demarcate the Vero and HFF-1 cell borders for cell-number quantitation. The viral-antigen signal can be compartmentalized within the cell mask. Cells that exhibited antigen signal higher than the selected threshold can be counted as positive for viral infection. The ratio of virus-positive cells to total number of analyzed cells can be used to determine the percentage of infection for each well on the assay plates. The effect of antiviral drugs and/or mammalian protease inhibitors on the viral infection can be assessed as percentage of inhibition of infection in comparison to control wells. The combination of the mammalian protease inhibitor and the antiviral drug is expected to decrease the percentage of infection by about 5%, 10%, 15%, 20%, 25%, 30%, 35%, 40%, 45%, 50%, 55%, 60%, 65%, 70%, 75%, 80%, 85%, 90%, 95% or 100% or between about 2%-5%, about 5%-10%, about 10%-20%, about 20%-30%, about 30%-40%, about 40%-50%, about 50%-60%, about 60%-70%, about 70%-80%, about 80%-90%, about 90%-100%. The resultant cell number and percentage of infection can be normalized for each assay plate. Analysis of dose-response curve can be performed using GeneData Screener or similar software applying Levenberg-Marquardt algorithm for curve-fitting strategy. The curve-fitting process, including individual data point exclusion, will be pre-specified by default software settings. R2 value quantified goodness of fit and fitting strategy can be considered acceptable at R2 > 0.8.

### Additional Therapeutic Agents

In some embodiments, the drug combinations, i.e., antiviral drug and mammalian protease inhibitors of the disclosure can be combined with other therapeutic agents. Other therapeutic agents can include additional cathepsin inhibitors or protease inhibitors. Drug combinations of the disclosure can include, but are not limited to, for example, Drug combinations according to the disclosure can be, but are not limited to, for example: Relacatib (GSK-462795, SB-462795), in combination with T-705 (Avigan) for use in the treatment of arenavirus infections; Relacatib (GSK-462795, SB-462795), in combination with T-705 (Avigan) for use in the treatment of SARS-CoV-2 infections; MIV-711, in combination with T-705 (Favipiravir, Avigan) for use in treatment of SARS-CoV-2 and other coronaviruses such as MERS and SARS-CoV; AM-3701, MIV-701, MIV-710, MIV-711, NC-2300, ORG-219517 or Relacatib (GSK-462795, SB-462795), in combination with JNJ-64041575, JNJ-1575, ALS-008176, AL-8176 (Lumicitabine) for use in the treatment of RSV infections; AM-3701, MIV-701, MIV-710, MIV-711, NC-2300, ORG-219517 or Relacatib (GSK-462795, SB-462795), in combination with JNJ-64041575, JNJ-1575, ALS-008176, AL-8176 (Lumicitabine) for use in the treatment of SARS-CoV-2 infections; AM-3701, MIV-701, MIV-710, MIV-711, NC-2300, ORG-219517 or Relacatib (GSK-462795, SB-462795) or Odanacatib (MK-0822) or Balicatib (AAE581), in combination with JNJ-64041575, JNJ-1575, ALS-008176, AL-8176 (Lumicitabine) for use in the treatment of RSV infections; AM-3701, MIV-701, MIV-710, MIV-711, NC-2300, ORG-219517 or Relacatib (GSK-462795, SB-462795) in combination with T-705 (Avigan, Favipiravir) or BCX 4430 (Galidesivir) for use in the treatment of SARS-CoV-2 infections; AM-3701, MIV-701, MIV-710, MIV-711, NC-2300, ORG-219517 or Relacatib (GSK-462795, SB-462795), in combination with GS-5734 (Redmdesivir) in the treatment of SARS-CoV-2 infections; Odanacatib (MK-0822) or Balicatib (AAE581), in combination with GS-5734 (Remdesivir) in the treatment of SARS-CoV-2 infections; Relacatib (GSK-462795, SB-462795), in combination with T-705 (Avigan, Favipiravir) along with lopinavir/ritonavir (Kaletra) for use in the treatment of filovirus infections; Relacatib (GSK-462795, SB-462795), in combination with T-705 (Avigan), along with lopinavir/ritonavir (Kaletra) for use in the treatment of coronavirus infections such as SARS-CoV-2 infections; Relacatib (GSK-462795, SB-462795), in combination with T-705 (Avigan), along with lopinavir/ritonavir (Kaletra) for use in the treatment of coronavirus infections such as SARS-CoV-2 and other coronaviruses such as MERS and SARS-CoV; AM-3701, MIV-701, MIV-710, MIV-711, NC-2300, ORG-219517 or Relacatib (GSK-462795, SB-462795), in combination with JNJ-64041575, JNJ-1575, ALS-008176, AL-8176 (Lumicitabine) for use in the treatment of RSV infections; AM-3701, MIV-701, MIV-710, MIV-711, NC-2300, ORG-219517 or Relacatib (GSK-462795, SB-462795), in combination with BCX4430 (Galidesivir), T-705 (Avigan, Favipiravir) along with a protease inhibitor such as darunavir (Prezista) in the treatment of coronavirus infections such as SARS-CoV-2 infections; AM-3701, MIV-701, MIV-710, MIV-711, NC-2300, ORG-219517 or Relacatib (GSK-462795, SB-462795), in combination with GS-5734 (Remdesivir), along with a protease inhibitor such as darunavir (Prezista) in the treatment of coronavirus infections such as SARS-CoV-2 infections; Odanacatib (MK-0822) or Balicatib (AAE581), in combination with GS-5734 (Remdesivir), along with a protease inhibitor such as darunavir (Prezista) in the treatment of coronavirus infections such as SARS-CoV-2 infections.

### Disease-Modifying Anti-Rheumatic Drugs (DMARDs)

In some embodiments, compositions can include gold preparations. As used herein, the term gold preparations can include auranofin. In some embodiments, compositions can include penicillamine, which can include D-penicillamine. In some embodiments, compositions can include aminosalicylic acid preparations, which can include sulfasalazine, mesalazine, olsalazine, balsalazide. In some embodiments, compositions can include antimalarials, which can include chloroquine. In some embodiments, compositions can include pyrimidine synthesis inhibitors, which can include leflunomide. In some embodiments, compositions can include prograf.

### Anti-Cytokine Drug

In some embodiments, compositions can include anti-cytokine drugs. As used herein, anti-cytokine drugs can include TNF inhibitors such as etanercept, infliximab, adalimumab, certolizumab pegol, golimumab, PASSTNF-alpha, soluble TNF-alpha receptor, TNF-alpha binding protein, anti-TNF-alpha antibody. As used herein, anti-cytokine drugs can include interleukin-1 inhibitors, such as anakinra (interleukin-1 receptor antagonist), soluble interleukin-1 receptor and the like; interleukin-6 inhibitors such as tocilizumab (anti-interleukin-6 receptor antibody), anti-interleukin-6 antibody. As used herein, anti-cytokine drugs can include interleukin-10 drugs such as interleukin-10. As used herein, anti-cytokine drugs can include interleukin-12/23 inhibitors such as ustekinumab, briakinumab (anti-interleukin-12/23 antibody). As used herein, anti-cytokine drugs can include B cell activation inhibitors such as rituximab, belimumab and the like; co-stimulatory molecules-related protein preparations such as abatacept and the like; complement mediated inhibitors both synthetic and biologic.

In some embodiments, compositions can include anti-cytokine drugs such as MAPK inhibitors such as BMS-582949. As used herein, anti-cytokine drugs can include gene modulators; inhibitors of molecule involved in signal transduction, such as NF-kappa, NF-kappaB, IKK-1, IKK-2, AP-1. As used herein, anti-cytokine drugs can include cytokine and chemokine production inhibitors, receptor binding inhibitors such as iguratimod, tetomilast. As used herein, anti-cytokine drugs can include TNF-alpha converting enzyme inhibitors; interleukin-1 beta converting enzyme inhibitors such as VX-765. As used herein, anti-cytokine drugs can include interleukin-6 antagonists such as HMPL-004. As used herein, anti-cytokine drugs can include interleukin-8 inhibitors such as IL-8 antagonist, CXCR1 & CXCR2 antagonist, reparixin. As used herein, anti-cytokine drugs can include Chemokine antagonists such as CCR9 antagonist (CCX-282, CCX-025), MCP-1 antagonist. As used herein, anti-cytokine drugs can include interleukin-2 receptor antagonists such as denileukin, diftitox. As used herein, anti-cytokine drugs can include therapeutic vaccines such as TNF-alpha vaccine. As used herein, anti-cytokine drugs can include gene therapy drugs such as drugs promoting the expression of a gene having an anti-inflammatory action such as interleukin-4, interleukin-10, soluble interleukin-1 receptor, soluble TNF-alpha receptor. As used herein, anti-cytokine drugs can include antisense compounds such as ISIS-104838. Integrin inhibitor

In some embodiments, compositions can include integrin inhibitors such as natalizumab, vedolizumab, AJM300, TRK-170, E-600.

### Immunomodulator (Immunosuppressant)

In some embodiments, compositions can include immunomodulators such as cyclophosphamide, MX-68, atiprimod dihydrochloride, BMS-188667, CKD-461, rimexolone, cyclosporine, tacrolimus, gusperimus, azathiopurine, antilymphocyte serum, freeze-dried sulfonated normal immunoglobulin, erythropoietin, colony stimulating factor, interleukin, interferon, intravenous immunoglobulin, anti-thymocyte globulin, RSLV-132. Proteasome inhibitor

In some embodiments, compositions can include proteasome inhibitors such as bortezomib.

### JAK Inhibitor

In some embodiments, compositions can include JAK inhibitors such as tofacitinib.

### Steroids

In some embodiments, compositions can include steroids. As used herein, steroid can include dexamethasone, hexestrol, methimazole, betamethasone, triamcinolone, triamcinolone acetonide, fluocinonide, fluocinolone acetonide, predonisolone, methylpredonisolone, cortisone acetate, hydrocortisone, fluorometholone, beclomethasone dipropionate, estriol.

### Angiotensin Converting Enzyme Inhibitors

In some embodiments, compositions can include angiotensin converting enzyme inhibitors. As used herein, angiotensin converting enzyme inhibitors can include enalapril, captopril, ramipril, lisinopril, cilazapril, perindopril.

### Angiotensin II Receptor Antagonists

In some embodiments, compositions can include angiotensin II receptor antagonists. As used herein, angiotensin II receptor antagonists can include candesartan, candesartan cilexetil (TCV-116), valsartan, irbesartan, olmesartan, eprosartan.

### Diuretic Substances

In some embodiments, compositions can include a diuretic. As used herein, a diuretic can include hydrochlorothiazide, spironolactone, furosemide, indapamide, bendrofluazide, cyclopenthiazide.

### Cardiotonic Substances

In some embodiments, compositions can include a cardiotonic substance. As used herein, a cardiotonic substance can include digoxin, dobutamine.

### Beta Receptor Antagonists

In some embodiments, compositions can include a beta receptor antagonist. As used herein, a beta receptor antagonist can include carvedilol, metoprolol, atenolol.

### Ca Sensitizers

In some embodiments, compositions can include a Ca sensitizer. As used herein, a CA sensitizer can include MCC-135.

### Ca Channel Antagonists

In some embodiments, compositions can include Ca channel antagonists. As used herein, a Ca channel antagonist can include nifedipine, diltiazem, verapamil.

### Anti-Platelet Drug, Anticoagulator

In some embodiments, compositions can include an anti-platelet substance or anticoagulator. As used herein, an anti-platelet substance or anticoagulator can include heparin, aspirin, warfarin.

### HMG-CoA Reductase Inhibitors

In some embodiments, compositions can include an HMG-CoA reductase inhibitor. As used herein, an anti-platelet substance or anticoagulator can include atorvastatin, simvastatin.

### Other Substances

In some embodiments, compositions can include other substances which improve functionality of the antiviral drugs and/or mammalian protease inhibitors. As used herein, other substances can include T cell inhibitors, inosine monophosphate dehydrogenase (IMPDH) inhibitor mycophenolate mofetil. As used herein, other substances can include adhesion molecule inhibitor such as ISIS-2302, selectin inhibitor, ELAM-1, VCAM-1, ICAM-1. As used herein, other substances can include thalidomide, a combination of cathepsin inhibitor or a single cathepsin inhibitor, matrix metalloprotease (MMPs) inhibitor such as V-85546. As used herein, other substances can include glucose-6-phosphate dehydrogenase inhibitor, Dihydroorotate dehydrogenase (DHODH) inhibitor, phosphodiesterase IV (PDE IV) inhibitor such as roflumilast, CG-1088. As used herein, other substances can include a phospholipase A2 inhibitor, iNOS inhibitor such as VAS-203. As used herein, other substances can include microtubule stimulating compound such as paclitaxel. As used herein, other substances can include microtubule inhibitor such as reumacon. As used herein, other substances can include MHC class II antagonist, prostacyclin agonist such as iloprost. As used herein, other substances can include CD4 antagonist such as zanolimumab. As used herein, other substances can include CD23 antagonist, LTB4 receptor antagonist such as DW-1305. As used herein, other substances can include 5-lipoxygenase inhibitor such as zileuton. As used herein, other substances can include cholinesterase inhibitor such as galanthamine. As used herein, other substances can include a tyrosine kinase inhibitor such as Tyk2 inhibitor (WO 2010/142752). As used herein, other substances can include cathepsin B inhibitor. As used herein, other substances can include adenosine deaminase inhibitor such as pentostatin. As used herein, other substances can include osteogenesis stimulator, dipeptidylpeptidase inhibitor, collagen agonist, capsaicin cream, hyaluronic acid derivative synvisc (hylan G-F 20), orthovis. As used herein, other substances can include glucosamine sulfate, amiprilose. As used herein, other substances can include CD-20 inhibitors such as rituximab, ibritumomab, tositumomab, ofatumumab. As used herein, other substances can include BAFF inhibitors such as belimumab, tabalumab, atacicept, A-623. As used herein, other substances can include CD52 inhibitors such as alemtuzuma

In some embodiments, compositions can include other substances which improve functionality of the antiviral drug and/or the mammalian protease inhibitor. As used herein, other substances can include antiviral substances such as idoxuridine, acyclovir, vidarabine, gancyclovir. As used herein, other substances can include anti-HIV agents such as zidovudine, didanosine, zalcitabine, indinavir sulfate ethanolate, ritonavir.

The simultaneous combination of sub-optimal doses of the drug along with one or more protease inhibitor, for example one or more cathepsin inhibitor, achieves an increase in function or efficacy of the drug, wherein the increase is any increase of about 2% and above, or between about 2%-5%, about 5%-10%, about 10%-20%, about 20%-30%, about 30%-40%, about 40%-50%, about 50%-60%, about 60%-70%, about 70%-80%, about 80%-90%, about 90%-100%, about 100%-150%, about 150%-200%, about 200%-300%, about 300%-400%, about 400%-500%, about 500%-1000%, 1000%-5000%, about 5000%-7000%, about 7000%-10,000% or more, or about 0.001-fold to about 0.01 fold, about 0.05-fold to about 0.1-fold, about 0.1-fold to about 0.5-fold, about 0.5-fold to about 1-fold, about 1-fold to about 2-fold, about 3-fold to about 5-fold, about 5-fold to about 10-fold, about 10-fold to about 20-fold, about 20-fold to about 40-fold, about 50-fold to about 75-fold, about 80 fold to about 100-fold, or more, such that the effective dose is decreased for each drug mentioned in this disclosure. Effective dose is that that achieves 50% of the effect which is also termed Inhibitory Concentration 50% (IC₅₀) or Effective Concentration (EC₅₀) in assays in vitro, wherein the EC₅₀ is decreased by about 5% or more, or by about 5%-10%, about 10%-20%, about 20%-30%, about 30%-40%, about 40%-50%, about 50%-60%, about 60%-70%, about 70%-80%, about 80%-90%, about 90%-100%, about 100%-150%, about 150%-200%, about 200%-300%, about 300%-400%, about 400%-500%, about 500%-1000%, 1000%-5000%, about 5000%-7000%, about 7000%-10,000% or more. In this disclosure, “sub-optimal doses” refers to doses which do not reach EC₅₀ or IC₅₀.

The combination or mixture of the present disclosure can be used together with other drugs for the prophylaxis or treatment of various diseases. For example, when the combination or mixture of the present disclosure is used as an antiviral therapy, it can be used together with the following drugs:

### Non-Steroidal Anti-Inflammatory Drugs (NSAIDs)

In some embodiments, compositions of antiviral drug and/or mammalian protease inhibitors can include classical non-steroidal anti-inflammatory drugs (NSAID). As used herein, the term NSAID can include, but are not limited to, alcofenac, aceclofenac, sulindac, tolmetin, etodolac, fenoprofen, thiaprofenic acid, meclofenamic acid, meloxicam, tenoxicam, lomoxicam, nabumeton, acetaminophen, phenacetin, ethenzamide, sulpyrine, antipyrine, migrenin, aspirin, mefenamic acid, flufenamic acid, diclofenac sodium, loxoprofen sodium, phenylbutazone, indomethacin, ibuprofen, ketoprofen, naproxen, oxaprozin, flurbiprofen, fenbufen, pranoprofen, floctafenine, piroxicam, epirizole, tiaramide hydrochloride, zaltoprofen, gabexate mesylate, ulinastatin, colchicine, probenecid, sulfinpyrazone, benzbromarone, allopurinol, sodium aurothiomalate, hyaluronate sodium, sodium salicylate, morphine hydrochloride, salicylic acid, atropine, scopolamine, morphine, pethidine, levorphanol, oxymorphone or a salt thereof and the like.

In some embodiments, compositions can include cyclooxygenase inhibitors. As used herein the term cyclooxygenase inhibitors can include, but are not limited to, (COX-1 selective inhibitors, COX-2 selective inhibitors, salicylic acid derivatives (e.g., celecoxib, aspirin), etoricoxib, valdecoxib, diclofenac, indomethacin, loxoprofen and the like.

In some embodiments, compositions can include Nitric oxide-releasing NSAIDs.

### Mechanism of Action

Although applicant is not bound by a mechanism, it is believed that the antiviral drugs and/or mammalian protease inhibitors of the disclosure are useful for treating enveloped viral infection by interfering with the critical role by cathepsins of proteolysis of the GP1 glycoprotein subunit to trigger membrane fusion and cell entry. The specific data presented herein suggest that GP1 proteolysis is a multistep process.

Changes in the integrated systemic concentrations over time are indicated by area under the curve (AUC) or Cₘₐₓ, both parameters well known in the art. AUC is determined by plotting the serum or plasma concentration of a drug along the ordinate (Y-axis) against time along the abscissa (X-axis). Generally, the values for the AUC represent drug concentrations over time in units of mass-time/volume. When efficacy of a drug is being measured, the amount and form of the drug administered should be the same in both a) the administration of the drug in combination with a protease inhibitor, for example a cathepsin inhibitor, or b) the administration of the drug alone.

Clearance of a drug normally occurs from the liver and kidneys and it is assumed that only free and non-protein bound drugs are available for clearance. For hepatic clearance, passive diffusion through the lipid core of the hepatocyte membranes, available to lipophilic drugs, is augmented by sinusoidal carrier systems particularly for ionized molecules (anionic and cationic) of molecular weights of approximately 3-400. Likewise, other transporters on the canalicular face transport drugs or their metabolites into bile. This system has two separate processes, hepatic uptake and biliary excretion. With small sized lipophilic drugs that readily traverse membranes hepatic uptake is not a maj or factor, but with higher molecular weight compounds (above 500) and those containing considerable H-bonding hepatic uptake can become the key clearance process, even if metabolism occurs subsequent to this.

Low bioavailability can occur when a drug rapidly dissolves and readily crosses the intestinal membranes. This absorption tends to be complete, but absorption of orally administered drugs is not always complete. Before reaching the vena cava, a drug must move down the gastrointestinal tract and pass through the gut wall and liver, common sites of drug metabolism. Thus, a drug can be metabolized during first-pass metabolism before it can be measured in the systemic circulation. Many drugs have low oral bioavailability because of expensive first-pass metabolism.

Low bioavailability is most common with oral dosage forms of poorly water-soluble, slowly absorbed drugs. More factors can affect bioavailability when absorption is slow or incomplete than when it is rapid and complete. That is, slow or incomplete absorption leads to variable therapeutic responses. Slow absorption in the gastrointestinal tract also leads to increased acute and delayed-phase chemotherapy induced nausea and vomiting.

Insufficient time in the gastrointestinal tract is a common cause of low bioavailability. Ingested drug is exposed to the entire gastrointestinal tract for no more than one to two days and to the small intestine for only 2 to 4 hours. If the drug does not dissolve readily or cannot penetrate the epithelial membrane (e.g., if it is highly ionized and polar), time at the absorption site can be insufficient. In such cases, bioavailability tends to be highly variable as well as low. Age, sex, activity, genetic phenotype, stress, disease, or previous gastrointestinal surgery can affect drug bioavailability.

Reactions that compete with absorption can reduce bioavailability. They include complex formation, hydrolysis by gastric acid or digestive enzymes, conjugation in the gut wall, absorption of other drugs and metabolism by luminal micro flora.

Assessment of bioavailability from plasma concentration-time data usually involves determining maximum peak concentration, the time at which maximum peak plasma drug concentration occurs, and the area under the plasma concentration time curve (AUC). The plasma drug concentration increases with the extent of absorption. The peak is reached when the drug elimination rate equals absorption rate. AUC is the most reliable measure of bioavailability. It is directly proportional to the total amount of unchanged drug that reaches the systemic circulation.

Drug products can be considered bioequivalent in extent and rate of absorption if their plasma level curves are essentially super imposable. Drug products that have similar AUCs but differently shaped plasma level curves are equivalent in extent but differ in their absorption rate-time profiles.

Absorption occurs by one of three methods, either passive diffusion, active transport or facilitated active transport. Passive diffusion is simply the passage of molecules across the mucosal barrier until the concentration of molecules reaches osmotic balance on both sides of the membrane. In active transport the molecule is actively pumped across the mucosa. In facilitated transport, a carrier generally a protein, is required to convey the molecule across the membrane for absorption.

### III. Methods

The present disclosure provides methods of use related to the compositions comprising antiviral drugs and/or mammalian protease inhibitors described herein. In some embodiments, the methods described herein can include a method of inhibiting the replication of a virus. Such methods can include contacting the virus with the compositions of the disclosure. As a non-limiting example the compositions of the disclosure can include mammalian protease inhibitors. The methods can involve contacting the virus with a mammalian protease inhibitor. In some embodiments, the replication of the virus is inhibited in vivo in a subject. In some aspects, the replication of the virus is inhibited in the presence of a cell or a population of cells infected by the virus. In some embodiments, the replication of the virus is inhibited by about 10%, 20%, 30%, 40%, 50%, 60%, 70%, 80% and/or 90%. In some embodiments the replication of the virus is inhibited by 5-15%, 10-20%, 15-25%, 20-30%, 25-35%, 30-40%, 35-45%, 40-50%, 45-55%, 50-60%, 55-65%, 60-70%, 65-75%, 70-80%, 75-85%, and/or 90-100%.

In some embodiments, the methods described herein can include a method of reducing the percentage of virus infected cells in a population. Such methods can include contacting the virus infected cells with the compositions e.g., antiviral drug and/or mammalian protease inhibitors of the disclosure. As a non-limiting example, the compositions of the disclosure can include mammalian protease inhibitors. The methods can involve contacting the virus infected cells with a mammalian protease inhibitor. In some embodiments, the percentage of the virus infected cells is reduced in vivo in a subject. In some embodiments, the percentage of virus infected cells in a population is reduced by about 10%, 20%, 30%, 40%, 50%, 60%, 70%, 80% and/or 90%. In some embodiments the percentage of virus infected cells in a population is reduced by 5-15%, 10-20%, 15-25%, 20-30%, 25-35%, 30-40%, 35-45%, 40-50%, 45-55%, 50-60%, 55-65%, 60-70%, 65-75%, 70-80%, 75-85%, and/or 90-100%.

The amount of the compositions of the disclosure to be utilized can be identified using methods described here. In some embodiments, the concentration of the protease inhibitors is determined using an MTS assays and cytotoxicity assays described herein or any other methods known in the art. Assay output can be analyzed to determine EC₅₀ (50% inhibition of virus replication), EC₉₀ (90% inhibition of virus replication), EC₉₅ (95% inhibition of virus replication), CC₅₀ (50% cytotoxicity), CC₉₅ (95% cytotoxicity). In some embodiments, the selectivity index (SI) for each protease inhibitor is determined by dividing the CC₅₀ by the EC₅₀.

In some embodiments, the methods of the disclosure selectivity index (SI) for each protease inhibitor for each virus infection can be determined. In some embodiments, the SI is determined for coronavirus. In one aspect, the SI can be at least 300, at least 400, at least 500, at least 600, at least 700, at least 800, at least 900 or more.

In one embodiment of the disclosure, the methods described herein correspond to a subject or patient or infected by a virus, such as RNA or DNA viruses which are pathogenic to humans and animals.

In some embodiments, the present disclosure provides antiviral drugs and/or mammalian protease inhibitors and compositions can be used to reduce the coronavirus infection levels in a subject. Such methods can include providing the compositions of the disclosure to a subject in need. The levels of coronavirus infection can be measured in the subject by any methods known in the art, such as, but not limited to, measuring coronavirus antigen levels, measuring anti-coronavirus antibodies and/or measuring levels of coronavirus nucleic acids in the subject. Treatment with the compositions of the disclosure is expected to reduce the coronavirus infection levels.

In another embodiment of the disclosure, the subject has an infection by a Type I enveloped virus. In some embodiments, the compositions of the disclosure can be provided or administered to a subject with an infection associated with a Type I enveloped virus e.g. a filovirus. In still another embodiment, the compositions can be used in the treatment of or can be provided to a subject infected with a coronavirus, a filovirus such as an Ebola virus or a Marburg virus. In yet another embodiment, the compositions can be used in the treatment of or can be provided to a subject infected with a Type I enveloped virus such as an orthomyxovirus. In still another embodiment, the compositions can be used in the treatment of or can be provided to a subject infected with a Type I enveloped virus such as a paramyxovirus. In still another embodiment, the Type I enveloped virus is an Arenavirus.

In some embodiments, the compositions of the disclosure can be used to inhibit a virus and/or reduce the percentage of virus infected cells in a population of cells. In some aspects, the virus can be a coronavirus, an enterovirus, an adenovirus, a dengue virus, human immunodeficiency virus, a parainfluenza virus, a respiratory syncytial virus (RSV), a coxsackie virus, a rhinovirus, Measles, Influenza virus.

In some embodiments, the virus can be a virus in the family, Coronaviridae, or a virus in the sub-family Orthocoronavirinae, or a virus in the order Nidovirales, In some embodiments, the methods of the disclosure can be used to inhibit the replication of any coronavirus. In one embodiment, the virus can be a coronavirus. In some embodiments, the coronavirus can be a SARS-CoV-2 virus, SARS-CoV-1 virus, MERS-CoV virus, 229E virus, NL63 virus, OC43 virus, HKU1 virus, or variants thereof. As a non-limiting example, the virus can be a SARS-CoV-2 virus.

In one embodiment, the virus can be an enterovirus.

In an embodiment of the disclosure, the subject or patient has, or is at risk of, an infection by a virus. These viruses, such as RNA or DNA viruses, are pathogenic for humans and animals. In an embodiment of the disclosure, the subject has or is at risk of infection by a Type I enveloped virus. In an embodiment, the Type I enveloped virus is a filovirus. In an embodiment, the filovirus is an Ebola virus or a Marburg virus. In an embodiment, the Type I enveloped virus is an orthomyxovirus. In an embodiment, the Type I enveloped virus is a paramyxovirus. In an embodiment, the Type I enveloped virus is an Arenavirus.

In an embodiment of the disclosure, the subject has or is at risk of infection by a virus such as, but not limited to, filoviruses, flaviviruses such as hepatitis-C virus, bunyaviruses, poxvirus, arboroviruses such as Togaviruses, bunyaviruses, orthomyxoviridae, paramyxoviridae, poxviruses, herpesviruses, henipaviruses, hepadnaviruses, rhabdoviruses, bomaviruses, arteriviruses, papillomaviridae, human retroviruses, polyomaviridae, picomaviridae, coronaviruses, and adenoviridae.

In some embodiments, the disclosure provides for methods of treating infection by a virus of the family Filoviridae, a family of viruses with a single-stranded, unsegmented (-) sense RNA genome. Filoviruses can cause severe hemorrhagic fever in humans and non-human primates. So far, only two genuses of this virus family have been identified: Marburg and Ebola. Four species of Ebola virus have been identified: Cote d‘Ivoire (CI), Sudan (S), Zaire (Z), and Reston (R). The Reston subtype is the only known filovirus that is not known to cause fatal disease in humans; however, it can be fatal in monkeys.

The family Orthomyxoviridae can include, without limitation, influenza A virus, influenza B virus, influenza C virus, Thogotovirus, Dhori virus, and infectious salmon anemia virus. Influenza type A viruses can be divided into subtypes based on two proteins on the surface of the virus. These proteins are called hemagglutinin (HA) and neuraminidase (NA). There are 15 different HA subtypes and 9 different NA subtypes. Subtypes of influenza A virus are named according to their HA and NA surface proteins, and many different combinations of HA and NA proteins are possible. For example, an “H7N2 virus” designates an influenza A subtype that has an HA 7 protein and an NA 2 protein. Similarly an “H5N1” virus has an HA 5 protein and an NA 1 protein. Only some influenza A subtypes (i.e., H1N1, H2N2, and H3N2) are currently in general circulation among people. Other subtypes such as H5 N1 are found commonly in other animal species and in a small number of humans, where it is highly pathogenic. For example, H7N7 and H3N8 viruses cause illness in horses. Humans can be infected with influenza types A, B, and C. However, the only subtypes of influenza A virus that normally infect people are influenza A subtypes H1N1, H2N2, and H3N2 and recently, H5N1.

The family Paramyxoviridae can include, without limitation, human parainfluenza virus, human respiratory syncytial virus (RSV), Sendai virus, Newcastle disease virus, mumps virus, rubeola (measles) virus, Hendra virus, Nipah virus, avian pneumovirus, and canine distemper virus.

The family Rhabdoviridae can include, without limitation, rabies virus, vesicular stomatitis virus (VSV), Mokola virus, Duvenhage virus, European bat virus, salmon infectious hematopoietic necrosis virus, viral hemorrhagic septicaemia virus, spring viremia of carp virus, and snakehead rhabdovirus. The family Bomaviridae can include, without limitation, Boma disease virus.

The family Bunyaviridae can include, without limitation, Bunyamwera virus, Hantaan virus, Crimean Congo virus, California encephalitis virus, Rift Valley fever virus, and sandfly fever virus. The family Arenaviridae includes, without limitation, Old World Arenaviruses, such as Lassa virus (Lassa fever), Ippy virus, Lymphocytic choriomeningitis virus (LCMV), Mobala virus, and Mopeia virus and New World Arenaviruses, such as Junin virus (Argentine hemorrhagic fever), Sabia (Brazilian hemorrhagic fever), Amapari virus, Flexal virus, Guanarito virus (Venezuela hemorrhagic fever), Machupo virus (Bolivian hemorrhagic fever), Latino virus, Boliveros virus, Parana virus, Pichinde virus, Pirital virus, Tacaribe virus, Tamiami virus, and Whitewater Arroyo virus.

The arboviruses are a large group (more than 400) of enveloped RNA viruses that are transmitted primarily (but not exclusively) by arthropod vectors (mosquitoes, sand-flies, fleas, ticks, lice, etc). More recently, the designated Arborviruses have been split into four virus families, including the togaviruses, flaviviruses, arenaviruses and bunyaviruses.

As used herein, the term “togavirus” refers to members of the family Togaviridae, which includes the genuses Alphavirus (e.g., Venezuela equine encephalitis virus, Sindbis virus, which causes a self-limiting febrile viral disease characterized by sudden onset of fever, rash, arthralgia or arthritis, lassitude, headache and myalgia) and Rubivirus (e.g. Rubella virus, which causes Rubella in vertebrates).

Flaviviridae is a member of the family of (+)-sense RNA enveloped viruses. Flaviviridae includes flavivirus, Pestivirus, and Hepacivirus. Flavivirus genus including yellow fever virus, dengue fever virus, and Japanese encaphilitis (JE) virus. The Pestivirus genus includes the three serotypes of bovine viral diarrhea, but no known human pathogens. Genus Hepacivirus consists of hepatitis C virus and hepatitis C-like viruses. The Japanese encephalitis antigenic complex includes Alfuy, Japanese encephalitis, Kokobera, Koutango, Kunjin, Murray Valley encephalitis, St. Louis encephalitis, Stratford, Usutu, and West Nile viruses. West Nile virus is the most widespread of the flaviviruses, with geographic distribution including Africa and Eurasia. The genus Pestivirus has been divided into bovine viral diarrhea virus (BVDV), classical swine fever virus (CSFV), and border disease virus (BDV). The Hepacivirus genus includes the hepatitis C virus (HCV).

Arenaviridae is a member of the family of (-) sense RNA viruses. As used herein, the term “Arenavirus” refers to members of the genus Arenavirius, a family of viruses whose members are generally associated with rodent-transmitted disease in humans, including Lymphocytic choriomeningitis virus (LCMV), Lassa virus, Junin virus, which causes Argentine hemorrhagic fever, Machupo virus, which causes Bolivian hemorrhagic fever, Guanarito virus, which causes Venezuelan hemorrhagic fever, and Sabia, which causes Brazilian hemorrhagic fever. LCMV causes which causes lymphocytic choriomeningitis, a mild disease that is occasionally severe with hemorrhaging.

The Phlebovirus Rift valley fever virus produces an acute, flu-like illness and is transmitted by mosquitoes from animal reservoirs (e.g., sheep) to man. Sand fly fever is transmitted to man by Phlebotomous flies (sand-flies) and causes an acute, febrile illness characterized by fever, malaise, eye pain, and headache.

Hendra and Nipah virus in the Henipavirus genus of the subfamily Paramyxovirinae are distinguished by fatal disease in both animal and human hosts.

Riboviria are all RNA viruses that replicate using RNA-dependent RNA polymerase. Examples of viruses that cause infections in humans include SARS-CoV-1, MERS-CoV, and SARS-CoV-2, 229E, NL63, OC43, KHU1.

Herpesviridae is a large family of DNA viruses that cause disease in animals, including humans. Herpesviruses include herpes simplex virus types 1 and 2, varicella-zoster virus, cytomegalovirus, Esptein-Barr virus, human herpesvirus 6 (variants A and B), human herpesvirus 7, and Kaposi’s sarcoma virus or human herpesvirus 8.

Hepadnaviridae is a family of DNA viruses that cause hepatitis in humans and animals. Hepadnaviridae include hepatitis B virus isolated from mammals or birds.

Papillomaviridae is a family of non-enveloped DNA viruses with over a hundred species of papillomaviruses including Alpha papillomavirus, Beta papillomavirus, Gamma papillomavirus, Mu papillomavirus and Nupapillomavirus. HPVs are most associated with cutaneous and genital legions, cervical carcinoma and recurrent respiratory papillomatosis, among other diseases.

Human retroviruses, including human T-cell leukemia virus (HTLV-1, 2, 3 and 4) and adult T-cell leukemia virus (ATLV) Human T-lymphotropic virus cause serious diseases in humans, including adult T-cell leukemia/lymphoma (ATL) and neurological disease (HTLV-associated myelopathy/tropical spastic paraparesis), uveitis, and rheumatic syndromes.

Polyomaviridae family of viruses are non-enveloped DNA viruses that cause disease in immunocompromised hosts. Human polyomaviruses BKV and JCV cause hemorrhagic cystitis and leukoencephalopathy. Merkel cell polyomavirus (MCPyV or MCV) shares some traits to plyomaviruses and is thought to be linked to Merkel Cell Carcinoma (MCC), a neuroendocrine cancer.

Poxviridae is a large family of DNA viruses including molluscipoxvirus, parapoxvirus (Orf virus, pseudocowpox virus, bovine popular stomatitis virus), Orthopoxvirus (cowpox virus, monkeypox virus, vaccinia virus, variola virus), Yatapoxvirus (tanapoxvirus, yaba monkey tumor poxvirus).

Picomaviridae are a family of viruses with single-stranded positive-sense RNA genomes and includes, without limitation, enteroviruses A through L, coxsackieviruses, echoviruses, polioviruses 1-3, and rhinoviruses A and B, hepatoviruses (Hepatitis A virus), cardioviruses (infect rodents, aphthoviruses (foot-and-mouth disease virus which infects cloven-hoofed animals and occasionally humans).

Adenoviridae is a family of double-stranded DNA viruses and include more than 100 antigenic types with human adenoviruses divided in subgenuses A-F and Serotypes 1-47, e.g. HAdV-B3, -E4, and -B7.

To test efficacy of the compositions against Marburg virus, HeLa cells can be seeded at 2,000 cells per well in a 384-well plate, and antiviral drugs and/or mammalian protease inhibitors can be added to the assay plates. Assay plates can be transferred to the BSL-4 suite and infected with 1 PFU per cell MARV, which can result in 50% to 70% of the cells expressing virus antigen in a 48-h period.

To test efficacy of the compositions against Sudan virus, HeLa cells can be seeded at 2,000 cells per well in a 384-well plate, and antiviral drugs and/or mammalian protease inhibitors can be added to the assay plates. Assay plates can be transferred to the BSL-4 suite and infected with 0.08 PFU SUDV per cell, which can result in 50% to 70% of the cells expressing virus antigen in a 48-h period.

To test efficacy against Lassa fever virus, HeLa cells can be seeded at 2,000 cells per well in a 384-well plate, and antiviral drugs and/or mammalian protease inhibitors can be added to the assay plates. Assay plates can be transferred to the BSL-4 suite and infected with 0.1 PFU per cell LASV, which can result in >60% of the cells expressing virus antigen in a 48-h period.

To test efficacy against Middle East respiratory syndrome, SARS-CoV-1, and SARS-CoV-2 Vero E6 cells can be seeded at 4,000 cells per well in a 384-well plate, and antiviral drugs and/or mammalian protease inhibitors can be added to the assay plates in a dose dependent manner. Assay plates can be transferred to the BSL-¾ suite and infected with 0.5 or other PFU per cell of MERS, SARS-CoV-1 and 2 virus, which can result in >70% of the cells expressing virus antigen in a 48-h period.

To test efficacy against Chikungunya virus, U2OS cells can be seeded at 3,000 cells per well in a 384-well plate, and antiviral drugs and/or mammalian protease inhibitors can be added to the assay plates. Assay plates can be transferred to the BSL suite and infected with 0.5 PFU per cell of CHIK, which can result in >80% of the cells expressing virus antigen in a 48-h period.

To test efficacy against Venezuelan equine encephalitis virus, HeLa cells can be seeded at 4,000 cells per well in a 384-well plate, and antiviral drugs and/or mammalian protease inhibitors can be added to the assay plates. Assay plates can be transferred to the BSL-4 suite and infected with 0.1 PFU per cell VEEV, which can result in >60% of the cells expressing virus antigen in a 20-h period.

In some embodiments, cytotoxicity assays can be conducted using antiviral drugs and/or mammalian protease inhibitors. HEp-2 (1.5 × 10³ cells per well) and MT-4 (2 × 10³ cells per well) cells can be plated in 384-well plates and incubated with the appropriate medium containing threefold serially diluted antiviral drugs and/or mammalian protease inhibitors ranging from 15 nM to 100,000 nM. PC-3 cells (2.5 × 10³ cells per well), HepG2 cells (4 × 10³ cells per well), hepatocytes (1 × 10⁶ cells per well), quiescent PBMCs (1 × 10⁶ cells per well), stimulated PBMCs (2 × 10⁵ cells per well), and RPTEC cells (1 × 10³ cells per well) can be plated in 96-well plates and incubated with the appropriate medium containing threefold serially diluted antiviral drugs and/or mammalian protease inhibitors ranging from 15 nM to 100,000 nM. Cells can be cultured for 4-5 days at 37° C. Following the incubation, the cells can be allowed to equilibrate to 25° C., and cell viability can be determined by adding Cell-Titer Glo viability reagent. The mixture can be incubated for 10 min, and the luminescence signal may be quantified using an Envision plate reader. Upon treatment with the combination of antiviral drug and mammalian protease inhibitor, cell viability can be increased by about 5%, 10%, 15%, 20%, 25%, 30%, 35%, 40%, 45%, 50%, 55%, 60%, 65%, 70%, 75%, 80%, 85%, 90%, 95% or 100% or between about 2%-5%, about 5%-10%, about 10%-20%, about 20%-30%, about 30%-40%, about 40%-50%, about 50%-60%, about 60%-70%, about 70%-80%, about 80%-90%, about 90%-100%, when compared to treatment with varying doses of the antiviral drug alone. Cell lines may be not authenticated and may be not tested for mycoplasma as part of routine use in cytotoxicity assays.

RNA synthesis by the RSV polymerase can be reconstituted in vitro using purified RSV L/P complexes and an RNA oligonucleotide template (Dharmacon), representing nucleotides 1-14 of the RSV leader promoter. RNA synthesis reactions can be performed as described previously, except that the reaction mixture can contain 250 µM guanosine triphosphate (GTP), 10 µM uridine triphosphate (UTP), 10 µM cytidine triphosphate (CTP), supplemented with 10 µCi [α-³²P] CTP, and either included 10 µM adenosine triphosphate (ATP) or no ATP. Under these conditions, the polymerase is able to initiate synthesis from the position 3 site of the promoter, but not the position 1 site. The NTP metabolite of GS-5734 can be serially diluted in DMSO and included in each reaction mixture at concentrations of 10, 30, or 100 µM as specified. RNA products can be analyzed by electrophoresis on a 25% polyacrylamide gel, containing 7 M urea, in Tris-taurine-EDTA buffer, and radiolabeled RNA products can be detected by autoradiography.

In some embodiments, RSV A2 polymerase inhibition assay can be performed. Transcription reactions can contain 25 µg of crude RSV RNP complexes in 30 µL of reaction buffer (50 mM Tris-acetate (pH 8.0), 120 mM potassium acetate, 5% glycerol, 4.5 mM MgCl₂, 3 mM DTT, 2 mM EGTA, 50 µg ml⁻¹ BSA, 2.5 U RNasin, 20 µM ATP, 100 µM GTP, 100 µM UTP, 100 µM CTP, and 1.5 µCi [α-³²P]ATP (3,000 Ci mmol⁻¹). The radiolabeled nucleotide used in the transcription assay can be selected to match the nucleotide analogue being evaluated for inhibition of RSV RNP transcription.

To determine whether nucleotide analogues inhibited RSV RNP transcription, antiviral drugs and/or mammalian protease inhibitors can be added using a six-step serial dilution in fivefold increments. After a 90-min incubation at 30° C., the RNP reactions can be stopped with 350 µl of Qiagen RLT lysis buffer, and the RNA can be purified using a Qiagen RNeasy 96 kit. Purified RNA can be denatured in RNA sample loading buffer at 65° C. for 10 min and run on a 1.2% agarose/MOPS gel containing 2 M formaldehyde. The agarose gel can be dried, exposed to a Storm phosphorimaging screen, and developed using a Storm phosphorimager.

### IV. Pharmaceutical Compositions

According to the present disclosure the antiviral drugs and/or mammalian protease inhibitors can be prepared as pharmaceutical compositions. It will be understood that such compositions necessarily comprise the antiviral drug and/or the mammalian protease inhibitor and, most often, a pharmaceutically acceptable excipient.

Relative amounts of the anti-viral drug, mammalian protease inhibitor, a pharmaceutically acceptable excipient, and/or any additional ingredients in a pharmaceutical composition in accordance with the present disclosure can vary, depending upon the identity, size, and/or condition of the subject being treated and further depending upon the route by which the composition is to be administered. For example, the composition can comprise between 0.1% and 99% (w/w) of the active ingredients. By way of example, the composition can comprise between 0.1% and 100%, e.g., between 0.5 and 50%, between 1-30%, between 5-80%, at least 80% (w/w) active ingredients.

In some embodiments, the pharmaceutical compositions described herein can comprise at least one anti-viral drug and at least one protease inhibitor. As a non-limiting example, the pharmaceutical compositions can contain an anti-viral drug and a cathepsin inhibitor.

Although the descriptions of pharmaceutical compositions provided herein are principally directed to pharmaceutical compositions which are suitable for administration to humans, it will be understood by the skilled artisan that such compositions are generally suitable for administration to any other animal, e.g., to non-human animals, e.g. non-human mammals. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, rats, birds, including commercially relevant birds such as poultry, chickens, ducks, geese, and/or turkeys.

In some embodiments, compositions are administered to humans, human patients, or subjects.

The present disclosure provides methods of use of compositions of the disclosure in a subject. The methods can include methods for improving drug efficacy, drug toxicity and/or lowering drug dose in a subject.

In some embodiments, the present disclosure provides methods for improving the efficacy of a drug by combining the drug with one or more mammalian protease inhibitors. Such combinations can increase function or efficacy of the drug, by about 2% and above, or between about 2%- 5%, about 5%-10%, about 10%-20%, about 20%-30%, 10 about 30%-40%, about 40%-50%, about 50%-60%, about 60%-70%, about 70%-80%, about 80%-90%, about 90%- 100%, about 100%-150%, about 150%-200%, about 200%- 300%, about 300%-400%, about 400%-500%, about 500%-1000%, 1000%-5000%, about 5000%-7000%, about 7000%- 10,000% or more, or about 0.001-fold to about 0.01 fold, about 0.05-fold to about 0.1-fold, about 0.1- fold to about 0.5-fold, about 0.5-fold to about 1- fold, about 1-fold to about 2-fold, about 3-fold to about 5-fold, about 5-fold to about 10-fold, about 10-fold to about 20-fold, about 20-fold to about 40- fold, about 50-fold to about 75-fold, about 80 fold to about 100-fold, or more, such that the effective dose is decreased for each drug mentioned in this disclosure. As used herein, an effective dose can is one that achieves 50% of the effect (also termed Inhibitory Concentration 50% (IC₅₀) or Effective Concentration (EC₅₀)) in assays in vitro, wherein the EC₅₀ is decreased by about 5% or more, or by about 5%-10%, about 10%-20%, about 20%-30%, about 30%-40%, about 30 40%-50%, about 50%-60%, about 60%-70%, about 70%- 80%, about 80%-90%, about 90%-100%, about 100%-150%, about 150%-200%, about 200%-300%, about 300%-400%, about 400%-500%, about 500%-1000%, 1000%-5000%, about 5000%-7000%, about 7000%-10,000% or more. “Sub-optimal doses” can refer to doses which do not reach EC₅₀ or IC₅₀.

In some embodiments, the present disclosure provides methods for reducing the toxicity of a drug by combining the drug with one or more mammalian protease inhibitors. Such combinations can reduce the toxicity of the drug, by about 2% and above, or between about 2%-5%, about 5%-10%, about 10%-20%, about 20%-30%, 10 about 30%-40%, about 40%-50%, about 50%-60%, about 60%-70%, about 70%-80%, about 80%-90%, about 90%-100%, about 100%-150%, about 150%-200%, about 200%- 300%, about 300%-400%, about 400%-500%, about 500%- 1000%, 1000%-5000%, about 5000%-7000%, about 7000%-10,000% or more, or about 0.001-fold to about 0.01 fold, about 0.05-fold to about 0.1-fold, about 0.1- fold to about 0.5-fold, about 0.5-fold to about 1- fold, about 1-fold to about 2-fold, about 3-fold to about 5-fold, about 5-fold to about 10-fold, about 10-fold to about 20-fold, about 20-fold to about 40- fold, about 50-fold to about 75-fold, about 80 fold to about 100-fold, or more, such that the toxicity is decreased for each drug mentioned in this disclosure.

The compositions of the disclosure can be used in the treatment of or can be provided to a subject infected with a virus of the family Coronaviridae. Coronaviridae is a family of positive strand RNA viruses and include many of the human pathogenic CoVs such as SARS-CoV-2, SARS-CoV-1 or MERS.

In some embodiments, the present disclosure provides a method for prophylaxis or treatment of a disease, comprising administering Relacatib (GSK-462795, SB- 462795), wherein the disease is caused by a Orthomyxoviridae, influenza A virus, influenza B virus, influenza C virus, Thogotovirus, Dhori virus, infectious salmon anemia virus, Paramyxoviridae, parainfluenza virus, Sendai virus, Newcastle disease virus, mumps virus, rubeola (measles) virus, Hendra virus, avian pneumovirus, canine distemper virus, Rhabdoviridae rabies virus, vesicular stomatitis virus (VSV), Mokola virus, Duvenhage virus, European bat virus, salmon infectious hematopoietic necrosis virus, viral hemorrhagic septicaemia virus, spring viremia of carp virus, snakehead rhabdovirus, Bornaviridae, Borna disease virus, Bunyaviridae Bunyamwera virus, Crimean Congo virus, California encephalitis virus, Rift Valley fever virus, sandfly fever virus, Ippy virus, Lymphocytic choriomeningitis virus (LCMV), Mobala virus, Mopeia virus, New World Arenaviruses, Junin virus (Argentine hemorrhagic fever), Sabia (Brazilian hemorrhagic fever), Amapari virus, Flexal virus, Guanarito virus (Venezuela hemorrhagic fever), Machupo virus (Bolivian hemorrhagic fever), Latino virus, Boliveros virus, Parana virus, Pichinde virus, Pirital virus, Tacaribe virus, Tamiami virus, Whitewater Arroyo virus, arboviruses togaviruses, Sindbis virus, Rubivirus, Rubella virus, Flaviviridae, flavivirus, Pestivirus, Hepacivirus, yellow fever virus, dengue fever virus, and Japanese encaphilitis (JE) virus, Pestivirus, hepatitis C virus, hepatitis C-like viruses, Japanese encephalitis Alfuy, Japanese encephalitis, Kokobera, Koutango, Kunjin, Murray Valley encephalitis, St. Louis encephalitis, Stratford, Usutu, Eastern encephalitis virus, Pestivirus, bovine viral diarrhea virus (BVDV), classical swine fever virus (CSFV), border disease virus (BDV), Arenaviridae, Lymphocytic choriomeningitis virus (LCMV), Phlebovirus Rift valley fever virus, Hendra, Riboviria, coronaviruses, SARS-CoV-2, rhinovirus, an enterovirus, a poliovirus, and an adenovirus.

### Dosing and Administration

Compositions of the present disclosure can be administered to a human or other mammal in a safe and effective amount as described herein. These safe and effective amounts will vary according to the type and size of mammal being treated and the desired results of the treatment. Any of the various methods known by persons skilled in the art for packaging tablets, caplets, or other solid dosage forms suitable for oral administration, that will not degrade the components of the present disclosure, are suitable for use in packaging. The combinations can be packaged in glass and plastic bottles. Tablets, caplets, or other solid dosage forms suitable for oral administration can be packaged and contained in various packaging materials optionally including a desiccant, e.g. silica gel. Packaging can be in the form of unit dose blister packaging. For example, a package can contain one blister tray of drug and another blister tray of protease pills, tablets, caplets, or capsule. A patient would take one dose, e.g. a pill, from one tray and one from the other. Alternatively, the package can contain a blister tray of the co-formulated combination of drug and protease in a single pill, tablet, caplet or capsule. As in other combinations and packaging thereof, the combinations of the disclosure include physiological functional derivatives of drug and protease. The packaging material can also have labeling and information related to the formulation printed thereon.

For any compound described herein the therapeutically effective amount can be initially determined from preliminary in vitro studies and/or animal models. A therapeutically effective dose can also be determined from human data for inhibitors which have been tested in humans and for compounds which are known to exhibit similar pharmacological activities, such as other related active agents. For instance, many cathepsin inhibitors have been extensively studied. The applied dose can be adjusted based on the relative bioavailability and potency of the administered compound. Adjusting the dose to achieve maximal efficacy based on the methods described above and other methods as are known in the art is well within the capabilities of the ordinarily skilled artisan.

The drug combination and other therapeutic agent can be administered simultaneously or sequentially. When the other therapeutic agents are administered simultaneously they can be administered in the same or separate formulation, but are administered at the same time. The other therapeutic agents are administered sequentially with one another and with the drug combination, when the administration of the other therapeutic agents and the drug combination is temporally separated. The separation in time between the administration of these compounds can be a matter of minutes or it can be longer. Other therapeutic agents include but are not limited to anti-viral vaccines and anti-viral agents. In some instances, the drug combination is administered with multiple therapeutic agents, i.e., 2, 3, 4 or even more different therapeutic agents.

In the above embodiments, the other therapeutic agent can be administered in the same dosage form or as a separate dosage form. When administered as a separate dosage form, the other therapeutic agent can be administered prior to, at the same time as, or following administration of the compound of the present disclosure or a formulation thereof.

While the dose of antiviral drug and/or mammalian protease inhibitors varies depending on the target disease, symptom, subject of administration, administration method and the like, for oral administration as a therapeutic agent, for example, the dose of the antiviral drug and/or mammalian protease inhibitors can be generally about 0.01-100 mg/kg body weight, e.g., 0.05-30 mg/kg body weight, and/or 0.5-10 mg/kg body weight, as one dose of the compound of the present disclosure , which is, for example, administered once to 3 times a day, on a weekly schedule, on a twice-weekly schedule and the like.

In some embodiments, the combination or mixture of the present disclosure or a formulation thereof is administered on a weekly schedule. In some embodiments, the drug or mammalian protease inhibitor, for example a cathepsin inhibitor, or a formulation thereof is administered on a weekly schedule. In some embodiments, the combination or mixture of the present disclosure or a pharmaceutical composition thereof is administered on days 1, 8, and 15 of a 28-day cycle. In some embodiments, the drug or mammalian protease inhibitor, for example a cathepsin inhibitor, or a formulation thereof is administered on days 1, 8, and 15 of a 28-day cycle.

In some embodiments, the combination or mixture of the present disclosure or a pharmaceutical composition thereof is administered on a twice weekly schedule. In some embodiments, the drug or the mammalian protease inhibitor, for example a cathepsin inhibitor, or a formulation thereof is administered on a twice-weekly schedule.

In some embodiments, the combination or mixture of the present disclosure or a formulation thereof is administered on days 1, 4, 8, and 11 of a 21-day cycle. In some embodiments, the drug or the mammalian protease inhibitor, for example a cathepsin inhibitor, or a formulation thereof is administered on days 1, 4, 8, and 11 of a 21-day cycle.

Additionally, an article of manufacture can contain a brochure, report, notice, pamphlet, or leaflet containing product information. This form of pharmaceutical information is referred to in the pharmaceutical industry as a “package insert.” A package insert can be attached to or included with a pharmaceutical article of manufacture. The package insert and any article of manufacture labeling provides information relating to the formulations. The information and labeling provides various forms of information utilized by health-care professionals and patients, describing the composition, its dosage and various other parameters required by regulatory agencies such as the United States Food and Drug Administration and other drug regulatory bodies such as EMA (European Medical Authorities).

### Administration

The composition of the present disclosure can be administered by any delivery route which results in a therapeutically effective outcome. These include, but are not limited to, enteral (into the intestine), gastroenteric, epidural (into the dura mater), oral (by way of the mouth), transdermal, intracerebral (into the cerebrum), intracerebroventricular (into the cerebral ventricles), epicutaneous (application onto the skin), intradermal (into the skin itself), subcutaneous (under the skin), nasal administration (through the nose), intravenous (into a vein), intravenous bolus, intravenous drip, intra-arterial (into an artery), intramuscular (into a muscle), intracardiac (into the heart), intraosseous infusion (into the bone marrow), intrathecal (into the spinal canal), intraparenchymal (into brain tissue), intraperitoneal (infusion or injection into the peritoneum), intravesical infusion, intravitreal (through the eye), intracavernous injection (into a pathologic cavity) intracavitary (into the base of the penis), intravaginal administration, intrauterine, extra-amniotic administration, transdermal (diffusion through the intact skin for systemic distribution), transmucosal (diffusion through a mucous membrane), transvaginal, insufflation (snorting), sublingual, sublabial, enema, eye drops (onto the conjunctiva), ear drops, auricular (in or by way of the ear), buccal (directed toward the cheek), conjunctival, cutaneous, dental (to a tooth or teeth), electro-osmosis, endocervical, endosinusial, endotracheal, extracorporeal, hemodialysis, infiltration, interstitial, intra-abdominal, intra-amniotic, intra-articular, intrabiliary, intrabronchial, intrabursal, intracartilaginous (within a cartilage), intracaudal (within the cauda equine), intracisternal (within the cisterna magna cerebellomedularis), intracorneal (within the cornea), dental intracoronal, intracoronary (within the coronary arteries), intracorporus cavemosum (within the dilatable spaces of the corporus cavernosa of the penis), intradiscal (within a disc), intraductal (within a duct of a gland), intraduodenal (within the duodenum), intradural (within or beneath the dura), intraepidermal (to the epidermis), intraesophageal (to the esophagus), intragastric (within the stomach), intragingival (within the gingivae), intraileal (within the distal portion of the small intestine), intralesional (within or introduced directly to a localized lesion), intraluminal (within a lumen of a tube), intralymphatic (within the lymph), intramedullary (within the marrow cavity of a bone), intrameningeal (within the meninges), intramyocardial (within the myocardium), intraocular (within the eye), intraovarian (within the ovary), intrapericardial (within the pericardium), intrapleural (within the pleura), intraprostatic (within the prostate gland), intrapulmonary (within the lungs or its bronchi), intrasinal (within the nasal or periorbital sinuses), intraspinal (within the vertebral column), intrasynovial (within the synovial cavity of a joint), intratendinous (within a tendon), intratesticular (within the testicle), intrathecal (within the cerebrospinal fluid at any level of the cerebrospinal axis), intrathoracic (within the thorax), intratubular (within the tubules of an organ), intratumor (within a tumor), intratympanic (within the aurus media), intravascular (within a vessel or vessels), intraventricular (within a ventricle), iontophoresis (by means of electric current where ions of soluble salts migrate into the tissues of the body), irrigation (to bathe or flush open wounds or body cavities), laryngeal (directly upon the larynx), nasogastric (through the nose and into the stomach), occlusive dressing technique (topical route administration which is then covered by a dressing which occludes the area), ophthalmic (to the external eye), oropharyngeal (directly to the mouth and pharynx), parenteral, percutaneous, periarticular, peridural, perineural, periodontal, rectal, respiratory (within the respiratory tract by inhaling orally or nasally for local or systemic effect), retrobulbar (behind the pons or behind the eyeball), soft tissue, subarachnoid, subconjunctival, submucosal, topical, transplacental (through or across the placenta), transtracheal (through the wall of the trachea), transtympanic (across or through the tympanic cavity), ureteral (to the ureter), urethral (to the urethra), vaginal, caudal block, diagnostic, nerve block, biliary perfusion, cardiac perfusion, photopheresis, and spinal.

In some embodiments, compositions can be administered in a way which allows them to cross the blood-brain barrier, vascular barrier, or other epithelial barrier. The viral particles of the present disclosure can be administered in any suitable form, either as a liquid solution or suspension, as a solid form suitable for liquid solution or suspension in a liquid solution. The viral particles can be formulated with any appropriate and pharmaceutically acceptable excipient.

In some embodiments, the composition of the present disclosure can be delivered to a subject via a single route administration.

In some embodiments, the composition of the present disclosure can be delivered to a subject via a multi-site route of administration. A subject can be administered at 2, 3, 4, 5, or more than 5 sites.

In some embodiments, a subject can be administered the composition of the present disclosure using a bolus infusion.

In some embodiments, a subject can be administered the composition of the present disclosure using sustained delivery over a period of minutes, hours, or days. The infusion rate can be changed depending on the subject, distribution, formulation or another delivery parameter.

In some embodiments, the composition of the present disclosure can be delivered by oral administration. Non-limiting examples of oral administration include a digestive tract administration and a buccal administration. In some embodiments, the composition of the present disclosure can be delivered by intraocular delivery route. A non-limiting example of intraocular administration include an intravitreal injection. In some embodiments, the composition of the present disclosure can be delivered by intranasal delivery route. Non-limiting examples of intranasal delivery include administration of nasal drops or nasal sprays. In some embodiments, the composition can be delivered by systemic delivery. As a non-limiting example, the systemic delivery can be by intravascular administration. In some embodiments, the composition of the present disclosure can be administered to a subject by intraparenchymal administration. In some embodiments, the composition of the present disclosure can be administered to a subject by intramuscular administration. In some embodiments, the composition of the present disclosure is administered to a subject and transduce muscle of a subject. As a non-limiting example, the composition is administered by intramuscular administration. In some embodiments, the composition of the present disclosure can be administered to a subject by intravenous administration. In some embodiments, the composition of the present disclosure can be administered to a subject by subcutaneous administration. In some embodiments, the composition of the present disclosure can be administered to a subject by topical administration. In some embodiments, the composition can be delivered by direct injection into the brain. As a non-limiting example, the brain delivery can be by intrastriatal administration. In some embodiments, the composition can be delivered by more than one route of administration. As non-limiting examples of combination administrations, composition can be delivered by intrathecal and intracerebroventricular, or by intravenous and intraparenchymal administration.

### Dosing

The present disclosure additionally provides a method of delivering to a subject, including a mammalian subject, any of the above-described composition comprising administering to the subject said composition, or administering to the subject a formulation comprising said composition, or administering to the subject any of the described compositions, including pharmaceutical compositions.

When the individual components of the combination are administered separately they are generally each presented as a pharmaceutical formulation. The references hereinafter to formulations refer unless otherwise stated to formulations containing either the combination or a component compound thereof. It will be understood that the administration of the combination of the disclosure by means of a single patient pack, or patient packs of each formulation, within a package insert diverting the patient to the correct use of the disclosure is a desirable additional feature of this disclosure. The disclosure also includes a double pack comprising in association for separate administration, formulations of the drug and protease, or a physiologically functional derivative of either or both thereof. The combination therapies of the disclosure include: (1) a combination of drug and mammalian protease inhibitor or (2) a combination containing a physiologically functional derivative of either or both thereof.

For any compound described herein the therapeutically effective amount can be initially determined from preliminary in vitro studies and/or animal models. A therapeutically effective dose can also be determined from human data for inhibitors which have been tested in humans and for compounds which are known to exhibit similar pharmacological activities, such as other related active agents. For instance, many cathepsin inhibitors have been extensively studied. The applied dose can be adjusted based on the relative bioavailability and potency of the administered compound. Adjusting the dose to achieve maximal efficacy based on the methods described above and other methods as are well-known in the art is well within the capabilities of the ordinarily skilled artisan.

In some embodiments, the dose of the compositions of the disclosure can be determined based on the concentration of the protease inhibitor required to inhibit the replication of a virus or reduce the percentage of virus infected cells. The concentration of the protease inhibitor can be from about 1 × 10⁻¹² M to about 1×10⁻³ M, for example, from about 0.1 µM to about 50 µM. In some embodiments, the concentration of the protease inhibitor can be 0.01 µM -0.1 µM, 0.1 µM -1 µM, 1 µM -10 µM, 10 µM -100 µM. In some embodiments, the concentration of the protease inhibitor can be 0.1 µM, 0.3 µM, 1 µM, 3 µM, 10 µM or 30 µM.

The mammalian protease inhibitor can have an effective concentration (EC₅₀) of from about 0.25 µM to about 50 µM. In some embodiments, the effective concentration of the protease inhibitor can be about 0.01 µM -0.1 µM, 0.1 µM -1 µM, 1 µM -10 µM, 10 µM -100 µM. In some embodiments, the effective concentration (EC₅₀) of the protease inhibitor is from about 15 µM to about 30 µM. In some embodiments, the effective concentration (EC₅₀) of the protease inhibitor is from about 0.25 µM to about 0.5 µM.

The mammalian protease inhibitor can have an effective concentration (EC₉₀) of from about 0.25 µM to about 50 µM. In some embodiments, the effective concentration of the protease inhibitor can be about 0.01 µM -0.1 µM, 0.1 µM -1 µM, 1 µM -10 µM, 10 µM -100 µM. In some embodiments, the effective concentration (EC₉₀) of the protease inhibitor is from about 1 µM to about 100 µM. In some embodiments, the effective concentration (EC₉₀) of the protease inhibitor is from about 1 µM to about 3 µM.

While the dose of the antiviral drug and/or mammalian protease inhibitors varies depending on the target disease, symptom, subject of administration, administration method and the like, for oral administration as a therapeutic agent, for example, it is generally about 0.01-1000 mg/kg body weight. The dose can be 0.01-0.1 mg/kg, 0.1-1 mg/kg, 1-10 mg/kg, 10-100 mg/kg, 100-1000 mg/kg, 0.05-30 mg/kg body weight, 0.5-10 mg/kg body weight, as one dose of the of the antiviral drug and/or mammalian protease inhibitors of the present disclosure, which is, for example, administered once to 3 times a day, on a weekly schedule, on a twice-weekly schedule and the like. In some embodiments, the antiviral drug and/or mammalian protease inhibitors of the disclosure can be administered daily for one, two, three, four, five or more times per day. The dose can be 10, 20, 30, 40, 50, 60, 70, 80, 90, 100, 150, 200, 250, 300, 350, 400, 450, 500, 550, 600, 650, 700 mg/kg body weight.

In some embodiments, the dose can be 400 mg oral dosing of Balicatib (HB-121) which can result in plasma concentrations of Balicatib (HB-121) above EC₅₀ for 16-20 hrs. In some aspects, the dosing of 400 mg can be provided orally once or twice daily.

In some embodiments, the compositions of the present disclosure or a pharmaceutical composition thereof is administered on a weekly schedule. In some embodiments, the compositions are administered on a weekly schedule.

In some embodiments, the compositions of the present disclosure or a pharmaceutical composition thereof is administered on a twice-weekly schedule. In some embodiments, the compositions of the present disclosure, for example a cathepsin inhibitor, or a pharmaceutical composition thereof is administered on a twice-weekly schedule.

In some embodiments, the compositions of the present disclosure of the present disclosure or a pharmaceutical composition thereof is administered on days 1, 4, 8, and 11 of a 21-day cycle.

In some embodiments, the compositions of the present disclosure or a pharmaceutical composition thereof is administered in conjunction with another therapeutic modality.

In certain such embodiments, the other therapeutic modality is one that is normally administered to patients with the disease to be treated or prevented. In some such embodiments, the other therapeutic modality is radiotherapy or plasmapheresis or another therapeutic agent.

In the above embodiments, the other therapeutic modality can be administered in the same dosage form or as a separate dosage form. When administered as a separate dosage form, the other therapeutic agent can be administered prior to, at the same time as, or following administration of the compound of the present disclosure or a pharmaceutical composition thereof.

Pharmaceutical formulations containing the antiviral drug and/or the mammalian protease inhibitor can be in any form suitable for the intended method of administration. When used for oral use for example, tablets, troches, lozenges, aqueous or oil suspensions, dispersible powders or granules, emulsions, hard or soft capsules, syrups or elixirs can be prepared an in Remington’s Pharmaceutical Sciences (Mack Publishing Co., Easton, PA, which is incorporated herein by reference in its entirety.

Compositions intended for oral use can be prepared according to any method known to the art for the manufacture of pharmaceutical compositions and such compositions can contain one or more agents including antioxidants, sweetening agents, flavoring agents, coloring agents and preserving agents, in order to provide a palatable preparation. Tablets containing the antiviral drug and/or the mammalian protease inhibitor in admixture with non-toxic pharmaceutically acceptable excipient which are suitable for manufacture of tablets are acceptable. These excipients can be, for example, inert diluents, such as calcium or sodium carbonate, lactose, lactose monohydrate, croscarmellose sodium, povidone, calcium or sodium phosphate; granulating and disintegrating agents, such as maize starch, or alginic acid; binding agents, such as cellulose, microcrystalline cellulose, starch, gelatin or acacia; and lubricating agents, such as magnesium stearate, stearic acid or talc. Tablets can be uncoated or can be coated by known techniques including microencapsulation to delay disintegration and absorption in the gastrointestinal tract and thereby provide a sustained action over a longer period. For example, a time delay material such as glyceryl monostearate or glyceryl distearate alone or with a wax can be employed. Formulations for oral use can be also presented as hard gelatin capsules where the antiviral drug and/or the mammalian protease inhibitor is mixed with an inert solid diluent, for example pregelatinized starch, calcium phosphate or kaolin, or as soft gelatin capsules wherein the antiviral drug and/or the mammalian protease inhibitor is mixed with water or an oil medium, such as peanut oil, liquid paraffin or olive oil.

The present disclosure provides pharmaceutical formulations combining the drug (e.g., the antiviral drug), a mammalian protease inhibitor, or physiologically functional derivatives thereof, in a sufficiently homogenized form, and a method for using this pharmaceutical formulation. An object of the present disclosure is to utilize glidants to reduce the segregation of antiviral drug and/or the mammalian protease inhibitor in pharmaceutical compositions during pre-compression material handling. Formulations include those suitable for oral, rectal, nasal, topical (including transdermal, buccal and sublingual), vaginal or parenteral (including subcutaneous, intramuscular, intravenous and intradermal) administration. The formulations can conveniently be presented in unit dosage form and can be prepared by any methods well known in the art of pharmacy. Such methods represent a further feature of the present disclosure and include the step of bringing into association the antiviral drug and/or the mammalian protease inhibitor with the carrier, which constitutes one or more accessory ingredients, and maintaining chemical stability.

In general, the formulations are prepared by uniformly and intimately bringing into association the antiviral drug and/or the mammalian protease inhibitor with liquid carriers or finely divided solid carriers or both, and then if necessary shaping the product. Formulations of the present disclosure suitable for oral administration can be presented as discrete units such as capsules, caplets, cachets or tablets each containing a predetermined amount of the antiviral drug and/or the mammalian protease inhibitor; as a powder or granules; as a solution or a suspension in an aqueous or non-aqueous liquid; or as an oil-in-water liquid emulsion or a water-in-oil liquid emulsion. The antiviral drug and/or the mammalian protease inhibitor can also be presented as a bolus, electuary or paste. A tablet can be made by compression or molding, optionally with one or more accessory ingredients. Compressed tablets can be prepared by compressing in a suitable machine the antiviral drug and/or the mammalian protease inhibitor in a free-flowing form such as a powder or granules, optionally mixed with a binder (e.g. povidone, gelatin, hydroxypropyl methylcellulose), lubricant, inert diluent, preservative, disintegrant (e.g. sodium starch glycollate, cross linked povidone, cross-linked sodium carboxymethyl cellulose) surface-active or dispersing agent. Molded tablets can be made by molding a mixture of the powdered compound moistened with an inert liquid diluent in a suitable machine. The tablets can optionally be coated or scored and can be formulated so as to provide slow or controlled release of the antiviral drug and/or the mammalian protease inhibitor therein using, for example, cellulose ether derivatives (e.g., hydroxypropyl methylcellulose) or methacrylate derivatives in varying proportions to provide the desired release profile. Tablets can optionally be provided with an enteric coating, to provide release in parts of the gut other than the stomach. Formulations suitable for topical administration in the mouth include lozenges comprising the antiviral drug and/or the mammalian protease inhibitor in a flavored base, usually sucrose and acacia or tragacanth; pastilles comprising the antiviral drug and/or the mammalian protease inhibitor in an inert basis such as gelatin and glycerin, or sucrose and acacia; and mouthwashes comprising the antiviral drug and/or the mammalian protease inhibitor in a suitable liquid carrier.

Formulations for rectal administration can be presented as a suppository with a suitable base comprising, for example, cocoa butter or a salicylates. Topical administration can also be by means of a transdermal iontophoretic device. Formulations suitable for vaginal administration can be presented as pessaries, tampons, creams, gels, pastes, foams or spray formulations containing in addition to the antiviral drug and/or the mammalian protease inhibitor such carriers as are known in the art to be appropriate. Formulations suitable for penile administration for prophylactic or therapeutic use can be presented in condoms, creams, gels, pastes, foams or spray formulations containing in addition to the antiviral drug and/or the mammalian protease inhibitor such carriers as are known in the art to be appropriate. Pharmaceutical formulations suitable for rectal administration wherein the carrier is a solid can be presented as unit dose suppositories. Suitable carriers include cocoa butter and other materials commonly used in the art. The suppositories can be conveniently formed by admixture of the active combination with the softened or melted carrier(s) followed by chilling and shaping in molds. Formulations suitable for parenteral administration include aqueous and nonaqueous isotonic sterile injection solutions which can contain anti-oxidants, buffers, bacteriostats and solutes which render the formulation isotonic with the blood of the intended recipient; and aqueous and non-aqueous sterile suspensions which can include suspending agents and thickening agents; and liposomes or other microparticulate systems which are designed to target the compound to blood components or one or more organs. The formulations can be presented in unit-dose or multi-dose sealed containers, for example, ampoules and vials, and can be stored in a freeze-dried (lyophilized) condition requiring only the addition of the sterile liquid carrier, for example water for injection, immediately prior to use. Extemporaneous injection solutions and suspensions can be prepared from sterile powders, granules and tablets of the kind previously described.

Exemplary unit dosage formulations are those containing a daily dose or daily subdose of the antiviral drug, and/or the mammalian protease inhibitor, as hereinbefore recited, or an appropriate fraction thereof. It should be understood that in addition to the ingredients particularly mentioned above the formulations of this disclosure can include other agents conventional in the art having regard to the type of formulation in question, for example, those suitable for oral administration can include such further agents as sweeteners, thickeners and flavoring agents.

### Aqueous Suspensions

Aqueous suspensions of the disclosure contain the antiviral drug and/or mammalian protease inhibitor in admixture with excipients suitable for the manufacture of aqueous suspensions. Such excipients include a suspending agent, such as sodium carboxymethylcellulose, methylcellulose, hydroxypropyl methylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., 1polyoxyethylene sorbitan monooleate).

The aqueous suspension can also contain one or more preservatives such as ethyl or n-propyl p-hydroxybenzoate, one or more coloring agents, one or more flavoring agents and one or more sweetening agents, such as sucrose, sucralose or saccharin. Oil suspensions can be formulated by suspending the antiviral drug and/or the mammalian protease inhibitor in a vegetable oil, such as arachis oil, olive oil, sesame oil or coconut oil, or in a mineral oil such as liquid paraffin. The oral suspensions can contain a thickening agent, such as beeswax, hard paraffin or cetyl alcohol. Sweetening agents, such as those set forth above, and flavoring agents can be added to provide a palatable oral preparation. These compositions can be preserved by the addition of an antioxidant such as ascorbic acid, BHT, etc. Dispersible powders and granules of the disclosure suitable for preparation of an aqueous suspension by the addition of water provide the antiviral drug and/or the mammalian protease inhibitor in admixture with a dispersing or wetting agent, a suspending agent, and one or more preservatives. Suitable dispersing or wetting agents and suspending a-gents are exemplified by those disclosed above. Additional excipients, for example sweetening, flavoring and coloring agents, can also be present.

The combinations of the disclosure can conveniently be presented as a pharmaceutical formulation in a unitary dosage form. A convenient unitary dosage formulation contains the antiviral drug and/or the mammalian protease inhibitor in any amount from 1 mg to 1 g each, for example but not limited to, 10 mg to 300 mg. The synergistic effects of a drug in combination with a mammalian protease inhibitor can be realized over a wide ratio, for example 1:1,000 to 1,000: 1 (drug: mammalian protease inhibitor). In one embodiment, the ratio can range from about 1:10 to 10:1. In another embodiment, the weight/weight or concentration/concentration ratio of drug to mammalian protease inhibitor in a co-formulated combination dosage form, such as a pill, tablet, caplet or capsule will be about 1, i.e., an approximately equal amount of drug and mammalian protease inhibitor. In other exemplary co-formulations, there can be more or less drug than mammalian protease inhibitor. For example, 300 mg drug and 200 mg mammalian protease inhibitor can be co formulated in a ratio of 1.5:1 (drug: mammalian protease inhibitor). In one embodiment, each compound will be employed in the combination in an amount at which it exhibits antiviral activity when used alone. Other ratios and amounts of the compounds of said combinations are contemplated within the scope of the disclosure.

It will be appreciated by those skilled in the art that the amount of antiviral drug and/or the mammalian protease inhibitor in the combinations of the disclosure required for use in treatment will vary according to a variety of factors, including the nature of the condition being treated and the age and condition of the patient, and will ultimately be at the discretion of the attending physician or health care practitioner. The factors to be considered include the route of administration and nature of the formulation, the animal’s body weight, age and general condition and the nature and severity of the disease to be treated.

It is also possible to combine the antiviral drug and/or the mammalian protease inhibitor in a unitary dosage form for simultaneous or sequential administration with a third active ingredient. The three-part combination can be administered simultaneously or sequentially. When administered sequentially, the combination can be administered in two or three administrations. Third active ingredients can have drug enhancing activity, similar activity as the drug, for example having anti-viral activity and include protease inhibitors (PI), nucleoside reverse transcriptase inhibitors (NRTI), non-nucleoside reverse transcriptase inhibitors (NNRTI), and integrase 5 inhibitors, or additional protease inhibitors. Exemplary third active ingredients to be administered in combination with drug and protease inhibitor, and their physiological functional derivatives, are 5,6 dihydro-5-azacytidine 5-aza 2′deoxycytidine 5-azacytidine 5 5-yl-carbocyclic 2′- deoxyguanosine (BMS200,475) 9 (arabinofuranosyl)guanine; 9-(2′ deoxyribofuranosyl)guanine 9-(2′-deoxy 2 ′fluororibofuranosyl)-2,6-diaminopurine 9-(2′-deoxy 2′fluororibofuranosyl)guanine 9-(2′-deoxyribofuranosyl)-2,6-diaminopurine 9-(arabinofuranosyl)-2,6 diaminopurine Abacavir, Ziagen® Acyclovir, ACV; 9-(2- hydroxyethoxylinethyl)guanine Adefovir dipivoxil, Hepsera® amdoxivir, DAPD 15 Aniprenavir, Agenerase® araA; 9-0-D-arabinofuranosyladenine (Vidarabine) atazanivir sulfate (Reyataz®) AZT; 3′-azido-2′,3′-dideoxythymidine, Zidovudine, (Retrovir®) BHCG; (.-i-.)-(1 a, 2 b, 3 a)-9-[2,3-bis(hydroxymethyl)cyclobutyl]guanine BMS200,475; 5-yl-carbocyclic 2′-deoxyguanosine Buciclovir;, (R) 9-(3,4-dihydroxybutyl)guanine BvaraU; 1-]1-Darabinofuranosyl-E-5-(2-bromovinyl)uracil (Sorivudine) Calanolide A Capravirine CDG; carbocyclic 2′-deoxyguanosine Cidofovir, HPMI′C; (S)-9-(3-hydroxy-2-phosphonyhnethoxypropyl)cytosine Clevudine, L-FMAU; 2′-Fluoro-5-methyl4--arabino-furanosyluraciI Combivir® (lamivudine/zidovudine) Cytallene; [1-(4′-hydroxy-1′,2′-butadienyl)cytosine] 30 d4C; 3′- deoxy-2′,3′-didehydrocytidine DAPD; (-)-1-D-2,6-diaminopurine dioxolane ddA; 2′,3′-dideoxyadenosine ddAPR; 2,6-diaminopurine-2′,3′-dideoxyriboside ddC; 2′,3′-dideoxycytidine (Zalcitabine) ddI; 2′,3′- 5 dideoxyinosine, didanosine, (Videx®, Videx® EC) Delavirdine, Rescriptor® Didanosine, ddI, Videx®; 2′,3′-dideoxyinosine DXG; dioxolane guanosine E-5- (2-bromovinyl)-2′-deoxyuridine Efavirenz, Sustiva®D Enfuvirtide, Fuzeon® F-ara-A; fluoroarabinosyladenosine (Fludarabine) FDOC; (-)-1-D-5-fluoro- 1-12-(hydroxymethyl)-1,3-dioxolane]cytosine FEAU; 2′-deoxy-2′-fluoro--p3-Darabinofuranosy-5-ethyluraciI FIAC; 1-(2-deoxy-2-fluoro-p3-D-arabinofuranosyl)-5-iodocytosine 15 FIAU; 1-(2-deoxy-2-fluoro-p3-D-arabinofuranosyl)-5- iodouridine FLG; 2′,3′-dideoxy-3′-fluoroguanosine FLT; 3′-deoxy-3′-fluorothymuidine Fluclarabine; Fara-A; fluoroarabinosyladenosine FMAU; 2′-Fluoro-5-methy1-fp-L-arabino-furanosyluraciI FMdC Foscarnet; phosphonoformic acid, PFA FPMPA; 9-(3-fluoro-2-phosphonylmethoxypropyl)adenine Gancyclovir, GCV; 9-(1,3-dihydroxy-2-propoxymethyl)guanine GS-7340; 9-[R-24[[(S)-[[(S)-1-(isopropoxycarbonyl)ethyl]amino] phenoxyphosphinyllmethoxy]propyl adenine HPA; (S)-9-(3-hydroxy-2-phosphonylmethoxypropyl)adenine HPMPC; (S)-9-(3-hydroxy-2-phosphonylmeffioxypropyl)cytosine (Cidofovir) Hydroxyurea, Droxia® Indinavir, Crixivan®, Kaletra® (lopinavir/ritonavir) Lamnivudine, 3TC, Epivi T m ; (2R, 5S, cis)-4-amino-1-(2-hyclroxymethyl-1,3 oxathiolan-5-yl)-(1H)-pyiidin-2-one L-d4C; L-3′-deoxy-2′,3′-didehydrocytidine L-ddC; L-2′,3′-dideoxycytidine 5 L-Fd4C; L-3′-deoxy-2′,3′-didehydro-5-fluorocytidine L-FddC; L-2′,3′-dideoxy 5-fluorocytidine Lopinavir Nelfinavir, Viracept® Nevirapine, Viramune® Oxetanocin A; 9-(2-deoxy-2-hydroxymethy-3-D-erythro-oxetanosyl)adenine Oxetanocin G; 9-(2-deoxy-2-hydroxymethy-3-D-5 erythroxetanosyl)guanine Penciclovir PMEBDAP; 9-(2-phosphonylinethoxyethyl)-2,6-diaminopurine PMPA, tenofovir; (R)-9-(2-phosphonylmethoxypropyl)adenine PPA; phosphonoacetic acid Ribavirin; 1-1-Dribofuranosyl-,2,4-triazole-3-carboxamide Ritonavir, Norvir® Saquinavir, Invirase®, Fortovase® Sonivudine, BvaraU; l-J0-D-arabinofuranosy1-E-5-(2- bromovinyl)uracil Stavudine, d4T, Zerit®; 2′,3′-didehydro-3′-deoxythyinidine Trifluorothymidine, TFT; Trifluorothyinidine Trizivir® (abacavir sulfate/lamivudine/zidovudine) Vidarabine, araA; 9-1-D-arabinofuranosyladenine Zalcitabine, Hlivid®,ddC; 2′,3′-dideoxycytidine 25 Zidovudine, AZT, Retrovir®; 3-azido-2′,3′-dideoxythymdine Zonavir; 5-propynyl-1-arabinosyluracil. Another aspect of the present disclosure is a three-part combination comprising tenofovir DF, FTC, and 9-[(R)-2-[[(S)-[[(S) 1(isopropoxycarbonyl)ethyl]amino]phenoxyphosphinyl]methoxy]propyl]adenine, also designated herein as GS-7340, GS-5734 (Remdesivir), A1-8170, and JNJ-64041575, JNJ-1575, ALS-008176, A1-8176 (Lumicitabine).

A further aspect of the disclosure is a patient pack comprising drug, mammalian protease inhibitor, or a physiologically functional derivative of either of the combination and an information package or product insert containing directions on the use of the combination of the disclosure. Segregation of antiviral drug and/or the mammalian protease inhibitor in pharmaceutical powders and granulations is a widely recognized problem that can result in inconsistent dispersions of the antiviral drug and/or the mammalian protease inhibitor in final dosage forms. Some of the main factors contributing to segregation are particle size, shape and density. Segregation is particularly troublesome when attempting to formulate a single homogenous tablet containing ingredients having different densities and different particle sizes. Glidants are substances that have traditionally been used to improve the flow characteristics of granulations and powders by reducing interparticulate friction. See Lieberman, Lachman, & Schwartz, Pharmaceutical Dosage Forms: Tablets, Volume 1, p. 177-178 (1989), incorporated herein by reference.

Glidants are typically added to formulations immediately prior to tablet compression to facilitate the flow of granular material into the die cavities of tablet presses. Glidants include: colloidal silicon dioxide, asbestos free talc, sodium aluminosilicate, calcium silicate, powdered cellulose, microcrystalline cellulose, corn starch, sodium benzoate, calcium carbonate, magnesium carbonate, metallic stearates, calcium stearate, magnesium stearate, zinc stearate, stearowet C, starch, starch 1500, magnesium lauryl sulfate, and magnesium oxide. Glidants can be used to increase and aid blend composition homogeneity in formulations of drugs (U.S. Pat. No. 6113920). The novel compositions of the present disclosure can contain glidants to effect and maintain homogeneity of antiviral drug and/or the mammalian protease inhibitor during handling prior to tablet compression.

The present disclosure provides pharmaceutical formulations combining a drug, a mammalian protease inhibitor, or physiologically functional derivatives thereof, in a sufficiently homogenized form, and a method for using this pharmaceutical formulation. An object of the present disclosure is to utilize glidants to reduce the segregation of antiviral drug and/or the mammalian protease inhibitor in pharmaceutical compositions during pre-compression material handling. Another object of the present disclosure is to provide a pharmaceutical formulation combining the drug and mammalian protease inhibitor, or physiologically functional derivatives thereof, with a pharmaceutically acceptable glidant, resulting in a mixture characterized by a pharmaceutically acceptable measure of homogeneity. Formulations include those suitable for oral, rectal, nasal, topical (including transdermal, buccal and sublingual), vaginal or parenteral (including subcutaneous, intramuscular, intravenous and intradermal) administration. The formulations can conveniently be presented in unit dosage form and can be prepared by any methods well known in the art of pharmacy. Such methods represent a further feature of the present disclosure and include the step of bringing into association the antiviral drug and/or the mammalian protease inhibitor with the carrier, which constitutes one or more accessory ingredients, and maintaining chemical stability.

In general, the formulations are prepared by uniformly and intimately bringing into association the antiviral drug and/or the mammalian protease inhibitor with liquid carriers or finely divided solid carriers or both, and then if necessary shaping the product. Formulations of the present disclosure suitable for oral administration can be presented as discrete units such as capsules, caplets, cachets or tablets each containing a predetermined amount of the antiviral drug and/or the mammalian protease inhibitor; as a powder or granules; as a solution or a suspension in an aqueous or non-aqueous liquid; or as an oil-in water liquid emulsion or a water-in-oil liquid emulsion. The antiviral drug and/or the mammalian protease inhibitor can also be presented as a bolus, electuary or paste. A tablet can be made by compression or molding, optionally with one or more accessory ingredients. Compressed tablets can be prepared by compressing in a suitable machine the antiviral drug and/or the mammalian protease inhibitor in a free-flowing form such as a powder or granules, optionally mixed with a binder (e.g. povidone, gelatin, hydroxypropyl methylcellulose), lubricant, inert diluent, preservative, disintegrant (e.g. sodium starch glycollate, cross linked povidone, cross-linked sodium carboxymethyl cellulose) surface-active or dispersing agent. Molded tablets can be made by molding a mixture of the powdered compound moistened with an inert liquid diluent in a suitable machine. The tablets can optionally be coated or scored and can be formulated so as to provide slow or controlled release of the antiviral drug and/or the mammalian protease inhibitor therein using, for example, cellulose ether derivatives (e.g., hydroxypropyl methylcellulose) or methacrylate derivatives in varying proportions to provide the desired release profile. Tablets can optionally be provided with an enteric coating, to provide release in parts of the gut other than the stomach. Formulations suitable for topical administration in the mouth include lozenges comprising the antiviral drug and/or the mammalian protease inhibitor in a flavored base, usually sucrose and acacia or tragacanth; pastilles comprising the antiviral drug and/or the mammalian protease inhibitor in an inert basis such as gelatin and glycerin, or sucrose and acacia; and mouthwashes comprising the antiviral drug and/or the mammalian protease inhibitor in a suitable liquid carrier.

Formulations for rectal administration can be presented as a suppository with a suitable base comprising, for example, cocoa butter or salicylates. Topical administration can also be by means of a transdermal iontophoretic device. Formulations suitable for vaginal administration can be presented as pessaries, tampons, creams, gels, pastes, foams or spray formulations containing in addition to the antiviral drug and/or the mammalian protease inhibitor such carriers as are known in the art to be appropriate. Formulations suitable for penile administration for prophylactic or therapeutic use can be presented in condoms, creams, gels, pastes, foams or spray formulations containing in addition to the antiviral drug and/or the mammalian protease inhibitor such carriers as are known in the art to be appropriate. Pharmaceutical formulations suitable for rectal administration wherein the carrier is a solid can presented as unit dose suppositories. Suitable carriers include cocoa butter and other materials commonly used in the art. The suppositories can be conveniently formed by admixture of the active combination with the softened or melted carrier(s) followed by chilling and shaping in molds. Formulations suitable for parenteral administration include aqueous and nonaqueous isotonic sterile injection solutions which can contain anti-oxidants, buffers, bacteriostats and solutes which render the formulation isotonic with the blood of the intended recipient; and aqueous and non-aqueous sterile suspensions which can include suspending agents and thickening agents; and liposomes or other microparticulate systems which are designed to target the compound to blood components or one or more organs. The formulations can be presented in unit-dose or multi-dose sealed containers, for example, ampoules and vials, and can be stored in a freeze-dried (lyophilized) condition requiring only the addition of the sterile liquid carrier, for example water for injection, immediately prior to use. Extemporaneous injection solutions and suspensions can be prepared from sterile powders, granules and tablets of the kind previously described.

Exemplary unit dosage formulations are those containing a daily dose or daily subdose of the antiviral drug and/or the mammalian protease inhibitor, as hereinbefore recited, or an appropriate fraction thereof. It should be understood that in addition to the ingredients particularly mentioned above the formulations of this disclosure can include other agents conventional in the art having regard to the type of formulation in question, for example, those suitable for oral administration can include such further agents as sweeteners, thickeners and flavoring agents. The compounds of the combination of the present disclosure can be obtained in a conventional manner, known to those skilled in the art.

### Emulsions

The pharmaceutical compositions of the disclosure can also be in the form of oil in-water emulsions or liposome formulations. The oily phase can be a vegetable oil, such as olive oil or arachis oil, a mineral oil, such as liquid paraffin, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan monooleate, and condensation products of these partial esters with ethylene oxide, such as polyoxyethylene sorbitan monooleate. The emulsion can also contain sweetening and flavoring agents. Syrups and elixirs can be formulated with sweetening agents, such as glycerol, sorbitol or sucrose. Such formulations can also contain a demulcent, a preservative, a flavoring or a coloring agent.

### Injectable

The pharmaceutical compositions of the disclosure can be in the form of a sterile injectable preparation, such as a sterile injectable aqueous or oleaginous suspension. This suspension can be formulated according to the known art using those suitable dispersing or wetting agents and suspending agents which have been mentioned above. The sterile injectable preparation can also be a sterile injectable solution or suspension in a non-toxic parenterally acceptable diluent or solvent, such as a solution in 1,3-butane diol or prepared as a lyophilized powder. Among the acceptable vehicles and solvents that can be employed are water, Ringer’s solution and isotonic sodium chloride solution. In addition, sterile fixed oils can conventionally be employed as a solvent or suspending medium. For this purpose any bland fixed oil can be employed including synthetic mono- or diglycerides. In addition, fatty acids such as oleic acid can likewise be used in the preparation of injectables.

The pharmaceutical compositions of the disclosure can be injected parenterally, for example, intravenously, intraperitoneally, intrathecally, intraventricularly, intrasystemically, intracranially, intramuscularly or subcutaneously, or they can be administered by infusion techniques. They are best used in the form of a sterile aqueous solution which can contain other substances, for example, enough salts or glucose to make the solution isotonic with blood. The aqueous solutions should be suitably buffered (e.g., to a pH of from 3 to 9), if necessary. The preparation of suitable parenteral formulations under sterile conditions is readily accomplished by standard pharmaceutical techniques well known to those skilled in the art.

### Intranasal

The pharmaceutical compositions of the disclosure can also be administered intranasally or by inhalation and are conveniently delivered in the form of a dry powder inhaler or an aerosol spray presentation from a pressurized container or a nebulizer with the use of a suitable propellant, e.g., dichlorodifluoromethane, trichlorofluoromethane, dichlorotetrafluoroethane, a hydrofluoroalkane such as 1,1,1,2-tetrafluoroethane (HFC 134a), carbon dioxide or other suitable gas. In the case of a pressurized aerosol, the dosage unit can be determined by providing a valve to deliver a metered amount. The pressurized container or nebulizer can contain a solution or suspension of the composition, e.g. using a mixture of ethanol and the propellant as the solvent, which can additionally contain a lubricant, e.g. sorbitan trioleate.

### Oral

Capsules and cartridges (made, for example, from gelatin) for use in an inhaler or insufflator can be formulated to contain a powder mix of a pharmaceutical composition of the disclosure and a suitable powder base such as lactose or starch. Aerosol or dry powder formulations can be arranged so that each metered dose or “puff contains from 20 pg to 200 mg of a composition for delivery to the patient. The overall daily dose with an aerosol or nebulizer will be in the range of from 20 pg to 200 mg which can be administered in a single dose or, more usually, in divided doses throughout the day. The amount of antiviral drug and/or the mammalian protease inhibitor that can be combined with the carrier material to produce a single dosage form will vary depending upon the host treated and the particular mode of administration. For example, a time-release formulation intended for oral administration to humans can contain approximately 1 to 1000 mg of the antiviral drug and/or the mammalian protease inhibitor with an appropriate and convenient amount of carrier material which can vary from about 5 to about 95% of the total compositions (weight: weight).

The pharmaceutical composition can be prepared to provide easily measurable amounts for administration. For example, an aqueous solution intended for intravenous infusion can contain from about 3 to 500 mg of the antiviral drug and/or the mammalian protease inhibitor per milliliter of solution in order that infusion of a suitable volume at a rate of about 30 ml/hr can occur.

As noted above, formulations of the present disclosure suitable for oral administration can be presented as discrete units such as capsules, cachets or tablets each containing a predetermined amount of the antiviral drug and/or the mammalian protease inhibitor; as a powder or granules; as a solution or a suspension in an aqueous or non-aqueous liquid; or as an oil-in-water liquid emulsion or a water-in-oil liquid emulsion. The antiviral drug and/or the mammalian protease inhibitor can also be administered as a bolus, electuary or paste.

### V. Formulations

Formulations of the pharmaceutical compositions described herein can be prepared by any method known or hereafter developed in the art of pharmacology. As used herein the term “pharmaceutical composition” refers to compositions comprising the antiviral drug and/or the mammalian protease inhibitor and optionally one or more pharmaceutically acceptable excipients.

Formulations of the anti-viral, protease inhibitors, and pharmaceutical compositions described herein can be prepared by any method known or hereafter developed in the art of pharmacology. In general, such preparatory methods include the step of bringing the antiviral drug and/or the mammalian protease inhibitor into association with an excipient and/or one or more other accessory ingredients, and then, if necessary and/or desirable, dividing, shaping and/or packaging the product into a desired single- or multi-dose unit.

A pharmaceutical composition in accordance with the present disclosure can be prepared, packaged, and/or sold in bulk, as a single unit dose, and/or as a plurality of single unit doses. As used herein, a “unit dose” refers to a discrete amount of the pharmaceutical composition comprising a predetermined amount of the antiviral drug and/or the mammalian protease inhibitor. The amount of the antiviral drug and/or the mammalian protease inhibitor ingredient is generally equal to the dosage of the antiviral drug and/or the mammalian protease inhibitor ingredient which would be administered to a subject and/or a convenient fraction of such a dosage such as, for example, one-half or one-third of such a dosage.

Relative amounts of the antiviral drug and/or the mammalian protease inhibitor (e.g. anti-viral, protease inhibitor), the pharmaceutically acceptable excipient, and/or any additional ingredients in a pharmaceutical composition in accordance with the present disclosure can vary, depending upon the identity, size, and/or condition of the subject being treated and further depending upon the route by which the composition is to be administered. For example, the composition can comprise between 0.1% and 99% (w/w) of the antiviral drug and/or the mammalian protease inhibitor. By way of example, the composition can comprise between 0.1% and 100%, e.g., between 0.5 and 50%, between 1-30%, between 5-80%, or at least 80% (w/w) antiviral drug and/or the mammalian protease inhibitor.

### Excipients and Diluents

The anti-viral and protease inhibitor of the disclosure can be formulated using one or more excipients or diluents to (1) increase stability; (2) increase absorption; (3) permit the sustained or delayed release; or (4) alter the biodistribution (e.g., target the antiviral drugs and/or mammalian protease inhibitors to specific tissues or cell types).

In some embodiments, a pharmaceutically acceptable excipient can be at least 95%, at least 96%, at least 97%, at least 98%, at least 99%, or 100% pure. In some embodiments, an excipient is approved for use for humans and for veterinary use. In some embodiments, an excipient can be approved by United States Food and Drug Administration. In some embodiments, an excipient can be of pharmaceutical grade. In some embodiments, an excipient can meet the standards of the United States Pharmacopoeia (USP), the European Pharmacopoeia (EP), the British Pharmacopoeia, and/or the International Pharmacopoeia.

Excipients, as used herein, include, but are not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 21st Edition, A. R. Gennaro, Lippincott, Williams & Wilkins, Baltimore, MD, 2006; incorporated herein by reference in its entirety). The use of a conventional excipient medium can be contemplated within the scope of the present disclosure, except insofar as any conventional excipient medium can be incompatible with a substance or its derivatives, such as by producing any undesirable biological effect or otherwise interacting in a deleterious manner with any other component(s) of the pharmaceutical composition.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and/or combinations thereof.

### Inactive Ingredients

In some embodiments, formulations can comprise at least one inactive ingredient. As used herein, the term “inactive ingredient” refers to one or more agents that do not contribute to the activity of the active ingredient of the pharmaceutical composition included in formulations. In some embodiments, all, none or some of the inactive ingredients which can be used in the formulations of the present disclosure can be approved by the US Food and Drug Administration (FDA).

Formulations of the disclosure can also include one or more pharmaceutically acceptable salts. As used herein, “pharmaceutically acceptable salts” refers to derivatives of the disclosed antiviral drugs and/or mammalian protease inhibitors wherein the parent antiviral drugs and/or mammalian protease inhibitors is modified by converting an existing acid or base moiety to its salt form (e.g., by reacting the free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, acetic acid, adipate, alginate, ascorbate, aspartate, benzenesulfonate, benzene sulfonic acid, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent antiviral drugs and/or mammalian protease inhibitors formed, for example, from non-toxic inorganic or organic acids.

Solvates can be prepared by crystallization, recrystallization, or precipitation from a solution that includes organic solvents, water, or a mixture thereof. Examples of suitable solvents are ethanol, water (for example, mono-, di-, and tri-hydrates), N-methylpyrrolidinone (NMP), dimethyl sulfoxide (DMSO), N,N′-dimethylformamide (DMF), N,N′-dimethylacetamide (DMAC), 1,3-dimethyl-2-imidazolidinone (DMEU), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, benzyl benzoate, and the like. When water is the solvent, the solvate is referred to as a “hydrate.”

### Non-Steroidal Anti-Inflammatory Drugs (NSAIDs)

In some embodiments, formulations of antiviral drugs and/or mammalian protease inhibitors can include classical non-steroidal anti-inflammatory drugs (NSAID). As used herein, the term NSAID can include, but are not limited to, alcofenac, aceclofenac, sulindac, tolmetin, etodolac, fenoprofen, thiaprofenic acid, meclofenamic acid, meloxicam, tenoxicam, lornoxicam, nabumeton, acetaminophen, phenacetin, ethenzamide, sulpyrine, antipyrine, migrenin, aspirin, mefenamic acid, flufenamic acid, diclofenac sodium, loxoprofen sodium, phenylbutazone, indomethacin, ibuprofen, ketoprofen, naproxen, oxaprozin, flurbiprofen, fenbufen, pranoprofen, floctafenine, piroxicam, epirizole, tiaramide hydrochloride, zaltoprofen, gabexate mesylate, ulinastatin, colchicine, probenecid, sulfinpyrazone, benzbromarone, allopurinol, sodium aurothiomalate, hyaluronate sodium, sodium salicylate, morphine hydrochloride, salicylic acid, atropine, scopolamine, morphine, pethidine, levorphanol, oxymorphone or a salt thereof and the like.

In some embodiments, formulations of antiviral drugs and/or mammalian protease inhibitors can include cyclooxygenase inhibitors. As used herein the term cyclooxygenase inhibitors can include, but are not limited to, (COX-1 selective inhibitors, COX-2 selective inhibitors, salicylic acid derivatives (e.g., celecoxib, aspirin), etoricoxib, valdecoxib, diclofenac, indomethacin, loxoprofen and the like.

In some embodiments, formulations can include Nitric oxide-releasing NSAIDs.

### Salts

If a pharmaceutically acceptable salt of the combination or mixture of the disclosure or a citric acid ester thereof is utilized in these compositions, the salt can be derived from an inorganic or organic acid or base. For reviews of suitable salts, see, e.g., Berge et al, J. Pharm. Sci. 66:1-19 (1977) and Remington: The Science and Practice of Pharmacy, 20th Ed., ed. A. Gennaro, Lippincott Williams & Wilkins, 2000.

As used herein, non-limiting examples of suitable acid addition salts include the following: acetate, adipate, alginate, aspartate, benzoate, benzene sulfonate, bisulfate, butyrate, citrate, camphorate, camphor sulfonate, cyclopentanepropionate, digluconate, dodecylsulfate, ethanesulfonate, fumarate, lucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenyl-propionate, picrate, pivalate, propionate, succinate, tartrate, thiocyanate, tosylate and undecanoate.

As used herein, suitable base addition salts include, without limitation, ammonium salts, alkali metal salts, such as lithium, sodium and potassium salts; alkaline earth metal salts, such as calcium and magnesium salts; other multivalent metal salts, such as zinc salts; salts with organic bases, such as dicyclohexylamine, N-methyl-D-glucamine, t-butylamine, ethylene diamine, ethanolamine, and choline; and salts with amino acids such as arginine, lysine, and so forth.

The pharmaceutical composition comprises the combination, whether as separate antiviral drugs and/or mammalian protease inhibitors or as a mixture, of the present disclosure and a pharmaceutically acceptable carrier.

The term “pharmaceutically acceptable carrier” is used herein to refer to a material that is compatible with a recipient subject, e.g., a mammal, such as a human, and is suitable for delivering an active agent e.g., antiviral drug and/or the mammalian protease inhibitor to the target site without terminating the activity of the agent. The toxicity or adverse effects, if any, associated with the carrier can be commensurate with a reasonable risk/benefit ratio for the intended use of the active agent.

The terms “carrier”, “adjuvant”, or “vehicle” are used interchangeably herein, and include any and all solvents, diluents, and other liquid vehicles, dispersion or suspension aids, surface active agents, pH modifiers, isotonic agents, thickening or emulsifying agents, preservatives, solid binders, lubricants and the like, as suited to the particular dosage form desired. Remington: The Science and Practice of Pharmacy, 20th Ed., ed. A. Gennaro, Lippincott Williams & Wilkins, 2000 discloses various carriers used in formulating pharmaceutically acceptable compositions and known techniques for the preparation thereof, which is incorporated by reference herein, in its entirety. Except insofar as any conventional carrier medium is incompatible with the compound of the disclosure, such as by producing any undesirable biological effect or otherwise interacting in a deleterious manner with any other component(s) of the pharmaceutically acceptable composition, its use is contemplated to be within the scope of this disclosure. Some examples of materials which can serve as pharmaceutically acceptable carriers include, but are not limited to, ion exchangers, alumina, aluminum stearate, lecithin, serum proteins, such as human serum albumin, buffer substances such as phosphates, carbonates, magnesium hydroxide and aluminum hydroxide, glycine, sorbic acid, or potassium sorbate, partial glyceride mixtures of saturated vegetable fatty acids, water, pyrogen-free water, salts or electrolytes such as protamine sulfate, disodium hydrogen phosphate, potassium hydrogen phosphate, sodium chloride, and zinc salts, colloidal silica, magnesium trisilicate, polyvinyl pyrrolidone, polyacrylates, waxes, polyethylene-polyoxypropylene-block polymers, wool fat, sugars such as lactose, glucose, sucrose, and mannitol, starches such as corn starch and potato starch, cellulose and its derivatives such as sodium carboxymethyl cellulose, ethyl cellulose and cellulose acetate, powdered tragacanth; malt, gelatin, talc, excipients such as cocoa butter and suppository waxes, oils such as peanut oil, cottonseed oil, safflower oil, sesame oil, olive oil, corn oil and soybean oil, glycols such as propylene glycol and polyethylene glycol, esters such as ethyl oleate and ethyl laurate, agar, alginic acid, isotonic saline, Ringer’s solution, alcohols such as ethanol, isopropyl alcohol, hexadecyl alcohol, and glycerol, cyclodextrins such as hydroxypropyl beta-cyclodextrin and sulfobutylether beta-cyclodextrin, lubricants such as sodium lauryl sulfate and magnesium stearate, petroleum hydrocarbons such as mineral oil and petrolatum. Coloring agents, releasing agents, coating agents, sweetening, flavoring and perfuming agents, preservatives and antioxidants can also be present in the composition, according to the judgment of the formulator.

It is possible for the mammalian protease inhibitor and the drug of the combination to be administered alone and separately as monotherapies, or to administer them as a pharmaceutical co-formulation. A two-part or three-part combination can be administered simultaneously or sequentially. When administered sequentially, the combination can be administered in one, two, or three administrations. In some embodiments, two-part or three-part combinations are administered in a single pharmaceutical dosage form. In some embodiments, a two-part combination is administered as a single oral dosage form and a three-part combination is administered as two identical oral dosage forms.

It will be appreciated that the compounds of the combination can be administered: (1) simultaneously by combination of the compounds in a co-formulation or (2) by alternation, i.e., delivering the compounds serially, sequentially, in parallel or simultaneously in separate pharmaceutical formulations.

In alternation therapy, the delay in administering the second, and optionally a third active ingredient, should not be such as to lose the benefit of a synergistic therapeutic effect of the combination of the active ingredients. By either method of administration (1) or (2), ideally the combination should be administered to achieve peak plasma concentrations of each of the active ingredients. A one pill once-per-day regimen by administration of a combination co formulation can be feasible for some IV-positive patients. Effective peak plasma concentrations of the active ingredients of the combination will be in the range of approximately 0.001 pM to 10 uM. Optimal peak plasma concentrations can be achieved by a formulation and dosing regimen prescribed for a particular patient. It will also be understood that either active ingredient, or the physiologically functional derivatives of either thereof, whether presented simultaneously or sequentially, can be administered individually, in multiples, or in any combination thereof. In general, during alternation therapy (2), an effective dosage of each compound is administered serially, where in co-formulation therapy (1), effective dosages of two or more compounds are administered together.

When the individual components of the combination are administered separately they are generally each presented as a pharmaceutical formulation. The references hereinafter to formulations refer unless otherwise stated to formulations containing either the combination or a component compound thereof. It will be understood that the administration of the combination of the disclosure by means of a single patient pack, or patient packs of each formulation, within a package insert diverting the patient to the correct use of the disclosure is a desirable additional feature of this disclosure.

The combination can be formulated in a unit dosage formulation comprising a fixed amount of each active pharmaceutical ingredient for a periodic, e.g., daily, dose or sub-dose of the active ingredients. Pharmaceutical formulations according to the present disclosure comprise a combination according to the disclosure together with one or more pharmaceutically acceptable carriers or excipients and optionally other therapeutic agents.

### VI. Definitions

While the disclosure will be described in conjunction with the enumerated claims, it will be understood that they are not intended to limit the disclosure to those claims. On the contrary, the disclosure is intended to cover all alternatives, modifications, and equivalents, which can be included within the scope of the present disclosure as defined by the claims.

Unless otherwise defined, all technical and scientific terms used herein have the same meaning as commonly understood by one of ordinary skill in the art to which this disclosure belongs. Although suitable methods and materials are described below, methods and materials similar or equivalent to those described herein can be used in the practice of the present disclosure. In case of conflict, the patent specification, including definitions, will control. All materials, methods, and examples are illustrative only and are not intended to be limiting.

When tradenames are used herein, applicants intend to independently include the tradename product and the active pharmaceutical ingredient(s) of the tradename product.

About: The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%.

Absorption: “Absorption” rate is important because even when a drug is absorbed completely, it can be absorbed too slowly to produce a therapeutic blood level quickly enough or so rapidly that toxicity results from high drug concentrations given to achieve the therapeutic level after each dose. The simultaneous combination of sub-optimal doses from the drug along with one or more mammalian protease inhibitor, for example one or more cathepsin inhibitor, achieves an increase in function or efficacy of the drug, wherein the increase is any increase of about 2% and above, or between about 2%- 5%, about 5%-10%, about 10%-20%, about 20%-30%, 10 about 30%-40%, about 40%-50%, about 50%-60%, about 60%-70%, about 70%-80%, about 80%-90%, about 90%- 100%, about 100%-150%, about 150%-200%, about 200%- 300%, about 300%-400%, about 400%-500%, about 500%-1000%, 1000%-5000%, about 5000%-7000%, about 7000%- 10,000% or more, or about 0.001-fold to about 0.01 fold, about 0.05-fold to about 0.1-fold, about 0.1- fold to about 0.5-fold, about 0.5-fold to about 1- fold, about 1-fold to about 2-fold, about 3-fold to about 5-fold, about 5-fold to about 10-fold, about 10-fold to about 20-fold, about 20-fold to about 40-fold, about 50-fold to about 75-fold, about 80 fold to about 100-fold, or more, such that the effective dose is decreased for each drug mentioned in this disclosure. “Absorption” rate is important because even when a drug is absorbed completely, it can be absorbed too slowly to produce a therapeutic blood level quickly enough or so rapidly that toxicity results from high drug concentrations given to achieve the therapeutic level after each dose.

“Bioavailability” is the degree to which a pharmaceutically active agent e.g., antiviral drug and/or the mammalian protease inhibitor becomes available to the target tissue after the agent’s introduction into the body. Enhancement of the bioavailability of a pharmaceutically active agent can provide a more efficient and effective treatment for patients because, for a given dose, more of the pharmaceutically active agent will be available at the targeted tissue sites. The compounds of the combinations of the disclosure can be referred to as “active ingredients” or pharmaceutically active agents.”

“Bioequivalence” refers to chemical equivalents that, when administered to the same person in the same dosage regimen, result in equivalent concentrations of drug in blood and tissues.

“Chemical equivalence” refers to drug products that contain the same compound in the same amount and that meet current official standards. However, inactive ingredients in drug products can differ.

“Clearance” of drug occurs by perfusion of blood to the organs of extraction. “Extraction” refers to the proportion of drug presented to the organ which is removed irreversibly (excreted) or altered to a different chemical form (metabolism). Clearance (CL) is therefore calculated as the product of the flow of blood through the organ and proportion of the drug extracted by the organ.

As used herein, the term “effective amount,” “effective concentration,” or “effective dose” means an amount that is sufficient upon appropriate administration to a patient (a) to cause a detectable decrease in the severity of the disorder or disease state being treated; (b) to ameliorate or alleviate the patient’s symptoms of the disease or disorder; or (c) to slow or prevent advancement of, or otherwise stabilize or prolong stabilization of, the disorder or disease state being treated. It should also be understood that a specific dosage and treatment regimen for any particular patient will depend upon a variety of factors, including the activity of the specific compound employed, the age, body weight, general health, sex, and diet of the patient, time of administration, rate of excretion, drug combinations, the judgment of the treating physician, and the severity of the particular disease being treated.

As used herein, the “maximum tolerated dose” (MTD) is the highest possible but still tolerable dose level with respect to a pre-specified clinical limiting toxicity. In general, these limits refer to the average patient population. For instances in which there is a large difference between the MED and MTD, it is stated that the drug has a large therapeutic window. Conversely, if the range is relatively small, or if the MTD is less than the MED, then the pharmaceutical product will have little to no practical value.

The term “method” refers to manners, means, techniques, processes and procedures for accomplishing a given task including, but not limited to, those manners, means, techniques, processes and procedures either known to, or readily developed from known manners, means, techniques, processes and procedures by practitioners of chemistry and/or pharmacology.

As used herein, the “minimum effective dose” (MED) is defined as the lowest dose level of a pharmaceutical product that provides a clinically significant response in average efficacy, which is also statistically significantly superior to the response provided by the placebo.

The term “physiologically functional derivative” means a pharmaceutically active compound e.g., antiviral drug and/or the mammalian protease inhibitor with equivalent or near equivalent physiological functionality when administered in combination with another pharmaceutically active compound alone or in combination with another compound. As used herein, the term “physiologically functional derivative” includes any: physiologically acceptable salt, ether, ester, prodrug, solvate, stereoisomer including enantiomer, diastereomer or stereoisomerically enriched or racemic mixture, and any other compound, which upon administration to a recipient, is capable of providing (directly or indirectly) such a compound or an active metabolite or residue thereof.

The term “potentiating” effect as used herein refers to and enhancement of an effect or action of an agent, a drug, or a chemical. A potentiating agent can be a chemical, an agent or a drug that enhances or intensifies an effect or action of another agent, chemical or drug.

The term “prodrug” as used herein refers to any compound that when administered to a biological system generates the drug substance, i.e., active ingredient e.g., antiviral drug and/or the mammalian protease inhibitor, as a result of spontaneous chemical reaction(s), enzyme catalyzed chemical reaction(s), and/or metabolic chemical reaction(s). “Prodrug moiety” means a labile functional group which separates from the active inhibitory compound during metabolism, systemically, inside a cell, by hydrolysis, enzymatic cleavage, or by some other process (Bundgaard, Hans, “Design and Application of Prodrugs” in Textbook of Drug Design and Development (1991), P. Krogsgaard Larsen and H. Bundgaard, Eds. Harwood Academic Publishers, pp. 113-191).

“Side effects” or “toxicity” or “adverse drug reactions” of drugs are side effects which can minor, severe, quite severe, or disabling and reversible or irreversible. In medicine, a side effect is an adverse effect that is secondary to the one intended; an unintended, consequences of the use of a drug whether in the targeted or untargeted parts of the body.

As used herein, the term “subject” or “patient” is a mammal, and examples thereof include human, dog, cat, bovine, horse, swine, or human.

The terms “synergy” and “synergistic” mean that the effect achieved when the drug and compound are used together is greater than the sum of the effects that results from using the drug and the compound separately, i.e., greater than what would be predicted based on the two active ingredients e.g., antiviral drug and/or the mammalian protease inhibitor administered separately. A synergistic effect can be attained when the drug and compound are: (1) co-formulated and administered or delivered simultaneously in a combined formulation; (2) delivered by alternation or in parallel as separate formulations; or (3) by some other regimen. When delivered in alternation therapy, a synergistic effect can be attained when the drug and compound are administered or delivered sequentially, e.g. in separate tablets, pills or capsules, or by different injections in separate syringes. In general, during alternation therapy, an effective dosage of each active ingredient e.g., antiviral drug and/or the mammalian protease inhibitor is administered sequentially, i.e. serially, whereas in combination therapy, effective dosages of two or more active ingredients are administered together. A synergistic antiviral effect denotes an antiviral effect which is greater than the predicted purely additive effects of the individual active ingredients of the combination.

As used herein, the term “treatment” means treating a patient having, or at risk of developing or experiencing a recurrence of the relevant disorder being treated, including suppression of progression of the relevant disorder being treated.

“Therapeutic equivalence” refers to drug products that, when administered to the same person in the same dosage regimen, provide essentially the same therapeutic effect or toxicity. Bioequivalent products are expected to be therapeutically equivalent. Sometimes therapeutic equivalence can be achieved despite differences in bioavailability, for example when the therapeutic index is wide (ratio of maximum tolerated dose to the minimum effective dose).

The details of one or more embodiments of the disclosure are set forth in the accompanying description below. Any materials and methods similar or equivalent to those described herein can be used in the practice or testing of the present disclosure. Other features, objects and advantages of the disclosure will be apparent from the description. In the description, the singular forms also include the plural unless the context clearly dictates otherwise. Unless defined otherwise, all technical and scientific terms used herein have the same meaning as commonly understood by one of ordinary skill in the art to which this disclosure belongs. In the case of conflict, the present description will control.

In order that this disclosure be more fully understood, the following preparative and testing examples are set forth. These examples illustrate how to make or test specific compounds and are not to be construed as limiting the scope of the disclosure in any way.

Techniques for practicing the specific aspect of this disclosure will employ, unless otherwise indicated, conventional techniques of molecular biology, microbiology, and recombinant DNA manipulation and production, which are routinely practiced by one of skill in the art. See, e.g., Sambrook et al., Molecular cloning, a laboratory manual, second ed., vol. 1-3. (Cold Spring Harbor Laboratory, 1989), A Laboratory Manual, Second Edition; DNA Cloning, Volumes I and II (Glover, Ed. 1985); and Transcription and Translation (Hames & Higgins, Eds. 1984). Western blot analysis or Northern blot analysis or any other technique used for the quantification of transcription of a nucleotide sequence, the abundance of its mRNA its protein (see Short Protocols in Molecular Biology, Ausubel et al., (editors), John Wiley & Sons, Inc., 4.sup.th edition, 1999; Current Protocols in Molecular Biology, volume 1-3 (1994-1998). Ed. by Ausubel, F. M., Brent, R., Kunston, R. E., Moore, D. D., Seidman, J. G., Smith, J. A. and Struhl, K. Published by John Wiley and sons, Inc., USA, Greene Publish. Assoc. & Wiley Interscience), (Short Protocols in Molecular Biology, 1999, Ed. Ausubel et al., John Wiley & Sons, Inc., Unit 10.11) etc. The details of one or more embodiments of the disclosure are set forth in the accompanying description below. Other features, objects and advantages of the disclosure will be apparent from the description. In the description, the singular forms also include the plural unless the context clearly dictates otherwise. Unless defined otherwise, all technical and scientific terms used herein have the same meaning as commonly understood by one of ordinary skill in the art to which this disclosure belongs. In the case of conflict, the present description will control.

The embodiments illustratively described herein suitably can be practiced in the absence of any element or elements, limitation or limitations that are specifically or not specifically disclosed herein. Thus, for example, in each instance herein any of the terms “comprising,” “consisting essentially of,” and “consisting of” can be replaced with either of the other two terms, while retaining their ordinary meanings. The terms and expressions which have been employed are used as terms of description and not of limitation, and there is no intention that in the use of such terms and expressions of excluding any equivalents of the features shown and described or portions thereof, but it is recognized that various modifications are possible within the scope of the claims. Thus, it should be understood that although the present methods and compositions have been specifically disclosed by embodiments and optional features, modifications and variations of the concepts herein disclosed can be resorted to by those skilled in the art, and that such modifications and variations are considered to be within the scope of the compositions and methods as defined by the description and the appended claims.

Any single term, single element, single phrase, group of terms, group of phrases, or group of elements described herein can each be specifically excluded from the claims.

The present disclosure is further illustrated by the following non-limiting examples.

The present disclosure is further illustrated by the following non-limiting examples.

### EXAMPLES

### Example 1. Efficacy of Balicatib Against Viruses

The efficacy of Balicatib (also referred to herein as HB-121) against several viruses was tested. Many viruses use cathepsins to enter the host cells. Cathepsins e.g. cathepsin B also help activate viral proteins. Accordingly, Balicatib (HB-121) can be effective against viruses. Table 1 provides the list of these viruses tested. In Table 1, CPE indicates cytopathic effect; and IF indicates immunofluorescent assays.

TABLE 1

Virus Strains

| | | | |
|---|---|---|---|
| Virus | Strain | Positive Control | Assay Readout (duration) |
| Adenovirus (Type 5) | Type 5 | Anti-Ad5 | CPE (6 days) |
| Dengue virus, serotype 2 (DENV2) | New Guinea C | Ribavirin | CPE (5 days) |
| HIV-1 (IIIB) | IIIB | AZT | CPE (6 days) |
| Parainfluenza Type 3 | Type 3 C243 | Ribavirin | CPE (7 days) |
| Respiratory syncytial virus (RSV) | Long | TMC353121 | CPE (5 days) |
| Coxsackie (A7) (CoxA7) | A7 | Enviroxime | CPE (4 days) |
| Rhinovirus 14 (HRV 14) | 1059 | Enviroxime | CPE (4 days) |
| Measles | Edmonston | STK316629 | CPE (10 days |
| Influenza A (Flu A. Cal) | A/California/07/2009 | Zanamivir | CPE (3 days) |
| Influenza B (Flu B.Bris) | B/Brisbane/60/2008 | Zanamivir | CPE (3 days) |
| Enterovirus (EV-71(H)) | 71H | Enviroxime | CPE (4 days) |

Viruses and cells were mixed in the presence of Balicatib (HB-121) and incubated for the assay duration as specified in Table 1. Each virus was pre-titered such that control wells exhibited 85 to 95% loss of cell viability due to virus replication. An antiviral effect or cytoprotective effect was considered to be observed when therapeutic agent (Balicatib or positive control indicated in Table 1) prevented virus replication. A standardized plate format was used for cytoprotective assays. Each plate contained cell control wells (cells only), virus control wells (cells plus virus), therapeutic agent cytotoxicity wells (cells plus therapeutic agent only), therapeutic agent colorimetric control wells (therapeutic agent only), background control wells (media only), as well as experimental wells (therapeutic agent plus cells plus virus). Samples were evaluated for antiviral efficacy with triplicate measurements to determine cytotoxicity, if detectable. Table 2 shows a representative plate format for evaluating test therapeutic agent (Balicatib/ HB-121; labeled “Drug 1” in Table 2) at 6 concentrations (2-fold dilutions) using representative high-test concentrations of 50 µM and control therapeutic agent (labelled “Drug 2” in Table 2) at 6 concentrations (10-fold dilutions) using representative high-test concentration of 200 IU/mL. Interferon (IFN) was used as positive controls in the assays were noted as IU/mL (bioactivity defined by International Units/mL was used from the certificate of analysis information provided by the vendor, PBL Assay Science, Piscataway, NJ). In Table 2, fields labeled as “Drug 1” or “Drug 2” indicate Cells + Virus + Drug; fields labeled as “Tox 1” or “Tox 2” indicate Cells + Drug 1 or Drug 2, respectively (toxicity tested in duplicate); Cells labeled as “Color 1” or “Color 2” = Media + Drug 1 or Drug 2, respectively (colorimetric background, no cells).

TABLE 2

Plate Format for Viral Assays

| | 1 | 2 | 3 | 4 | 5 | 6 | 7 | 8 | 9 | 10 | 11 | 12 |
|---|---|---|---|---|---|---|---|---|---|---|---|---|
| A | Reagent Background Control Wells (Media plus MTS, no cells) | | | | | | | | | | | |
| B | Tox 1 1.56 µM | Cell Contr ol | Drug 1 Low-Test 1.56 µM | | | Tox 1 1.56 µM | Tox 2 0.002 IU/mL | Drug 2 Low-Test 0.002 IU/mL | | | Cell Control | Tox 2 0.002 IU/mL |
| C | Tox 1 3.13 µM | | Drug 1 3.13 µM | | | Tox 1 3.13 µM | Tox 2 0.02 IU/mL | Drug 2 0.02 IU/mL | | | | Tox 2 0.02 IU/mL |
| D | Tox 1 6.25 µM | | Drug 1 6.25 µM | | | Tox 1 6.25 µM | Tox 2 0.20 IU/mL | Drug 2 0.20 IU/mL | | | | Tox 2 0.20 IU/mL |
| E | Tox 1 12.5 µM | Virus Control | Drug 1 12.5 µM | | | Tox 1 12.5 µM | Tox 2 2.00 IU/mL | Drug 2 2.00 IU/mL | | | Virus Control | Tox 2 2.00 IU/mL |
| F | Tox 1 25 µM | | Drug 1 25 µM | | | Tox 1 25 µM | Tox 2 20.0 IU/mL | Drug 2 20.0 IU/mL | | | | Tox 2 20.0 IU/mL |
| G | Tox 1 50 µM | | Drug 1 High-Test 50 µM | | | Tox 1 50 µM | Tox 2 200 IU/mL | Drug 2 High-Test 200 IU/mL | | | | Tox 2 200 IU/mL |
| H | Color 1 50 µM | Color 1 25 µM | Color 1 12.5 µM | Color 1 6.25 µM | Color 1 3.13 µM | Color 1 1.56 µM | Color 2 200 IU/mL | Color 2 20.0 IU/m L | Color 2 2.00 IU/m L | Color 2 0.20 IU/m L | Color 2 0.02 IU/m L | Color 2 0.002 IU/mL |

MTS Staining for Cell Viability

At assay termination, the assay plates were stained with the soluble tetrazolium-based dye MTS (CellTiter®96 Reagent, Promega) to determine cell viability and quantify compound toxicity. MTS is metabolized by the mitochondrial enzymes of metabolically active cells to yield a soluble formazan product, allowing the rapid quantitative analysis of cell viability and compound cytotoxicity. This reagent is a stable, single solution that does not require preparation before use. At termination of the assay, 10-25 µL of MTS reagent was added per well (10% final concentration based on volume) and the microtiter plates were then incubated for 2-3 hours at 37° C., (except Rhinovirus assay plates, which were incubated at 33° C.) 5% CO₂ to assess cell viability. Adhesive plate sealers were used in place of the lids, the sealed plates were inverted several times to mix the soluble formazan product and the plate was read spectrophotometrically at 490/650 nm with a Molecular Devices SpectraMax i3 plate reader.

### Data Analysis of MTS Staining

Using an in-house computer program, the CPE data analysis was conducted and included the calculation of EC₅₀ (50% inhibition of virus replication), EC₉₅ (95% inhibition of virus replication), CC₅₀ (50% cytotoxicity), CC₉₅ (95% cytotoxicity) and selectivity index values (SI = CC/EC; also referred to as Antiviral Index or AI).

### Cytotoxicity Assay

Cells were seeded at 2,500 to 40,000 cells per well in 96-well plates in replication medium as specified in Table 3. The plates were incubated overnight at 37° C. and 5% CO₂. The following day, test compound was prepared in assay medium. Growth medium was removed from the plates and replaced with the prepared test compound. Each dilution of compound was tested in triplicate. Each toxicity plate contained the necessary cell control and compound color controls. After the assay readout (duration) as specified in Table 1, cell viability was determined using MTS dye reduction. In Table 3, DMEM indicates Dulbecco’s Modified Eagle’s Medium; FBS indicates Fetal Bovine Serum; and TPCK indicates N-p-Tosyl-L-phenylalanine chloromethyl ketone.

TABLE 3

Cells and Media used for Antiviral Evaluations

| | | |
|---|---|---|
| Virus | Cell Line | Assay Medium |
| Adenovirus (Type 5) | HeLa | DMEM+2% FB S |
| Dengue virus, serotype 2 | BHK-21 | DMEM+2% FB S |
| HIV-1 (IIIB) | MT-4 | R10 |
| Parainfluenza Type 3 (C243) | LLC-MK2 7.1 | DMEM+2% FB S |
| Respiratory syncytial virus | HEp-2 | DMEM+2% FB S |
| Cox (A7) | LLC-MK2 7.1 | DMEM+2% FB S |
| Rhinovirus 14 (assay conducted at 33° C.) | H1 HeLa | MEM+2%FBS² |
| Measles Virus (Edmonston) | MRC-5 | DMEM+2% FB S |
| Influenza A | MDCK | DMEM w/HEPES, 0.5 ug/mL TPCK treated Trypsin |
| Influenza B | MDCK | DMEM w/HEPES, 0.5 ug/mL TPCK treated Trypsin |
| Enterovirus | RD | DMEM+2% FB S |

### Analysis of Cytotoxicity Assay

The minimum inhibitory drug concentration that reduced plaque formation by 50% (EC₅₀) and the minimum drug concentration that inhibited cell growth by 50% (CC₅₀) were calculated. The selectivity index (SI) for each active compound was determined by dividing the CC₅₀ by the EC₅₀.

### Results

Balicatib (HB-121) was tested against multiple viruses such as Influenza strains listed in Table 1 and Table 3. Balicatib (HB-121) showed antiviral activity against Enterovirus 71 at about 20 µM EC₅₀. The results are shown in Table 4 below:

TABLE 4

Efficacy of Balicatib against Enterovirus 71

| | | | |
|---|---|---|---|
| DRUG: HB-121 | 25% | 50% | 95% |
| CC (µM) | > 30.0 | > 30.0 | > 30.0 |
| EC (µM) | 5.85 | 22.1 | >30.0 |
| Selectivity Index (SI) | >5.13 | >1.36 | N/A |

Balicatib (HB-121) was not effective against the other viruses listed in Table 1 at the concentrations tested.

### Example 2. Evaluation Assay for SARS-CoV

Calu-3 cells were seeded in 96 well plate. After 24 h, cells were washed and treated with various concentrations of Balicatib (HB-121 or compound 21 in this assay) or two other cathepsin inhibitors compounds ONO-5334 (compound 19), or Odanacatib (MK-0822 (compound 22) in a serum-free medium (n=6). The incubation of cells with PBS served as a negative control (n=6). After 4 h of treatment, cells were washed and replaced with serum-free media for infection with SARS-CoV-2 (strain: BEI_USA-WA1/2020) multiplicity of infection (MOI) of 0.01 for 1 h at 37° C. After infection, cells were washed and replaced with 5% FBS containing media. After 48 h post-infection, cells were fixed with 4% buffered paraformaldehyde (Electron Microscopy Sciences) for 15 min at room temperature. The fixed cells were washed with PBS then permeabilized in 0.1% Triton X100 PBS solution for 15 min then blocked in 3% BSA PBS solution. The cells were incubated with anti-S protein Rab (Sino Biological, PA, USA) at 1:1000 in the blocking solution overnight at 4° C., followed by incubation with 1:2000 diluted Alexa Fluor 488 conjugated secondary antibody (Thermo Fisher, MA, USA) for 1 h at room temperature. The cells were counterstained for nuclei with Hoechst 33342 (Thermo Fisher, MA, USA). The fluorescent images were captured by using an Operetta system. Total and virus-infected cells were counted by imaging and %infection was plotted. The compounds were blinded to the testers in the experiments.

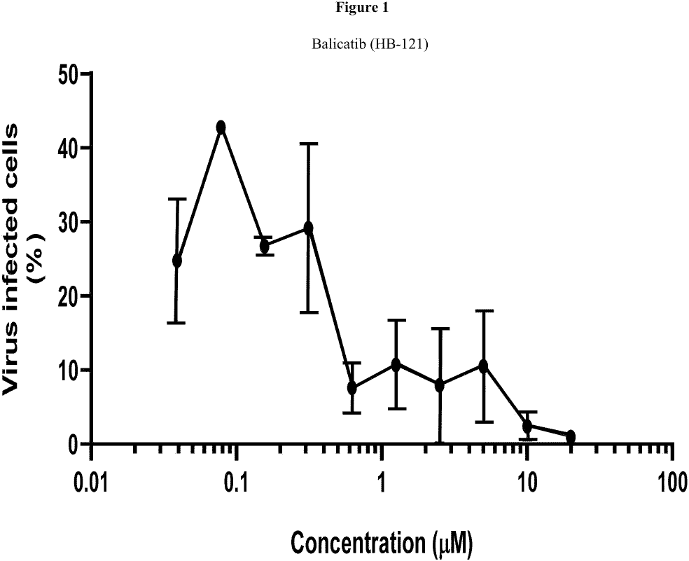

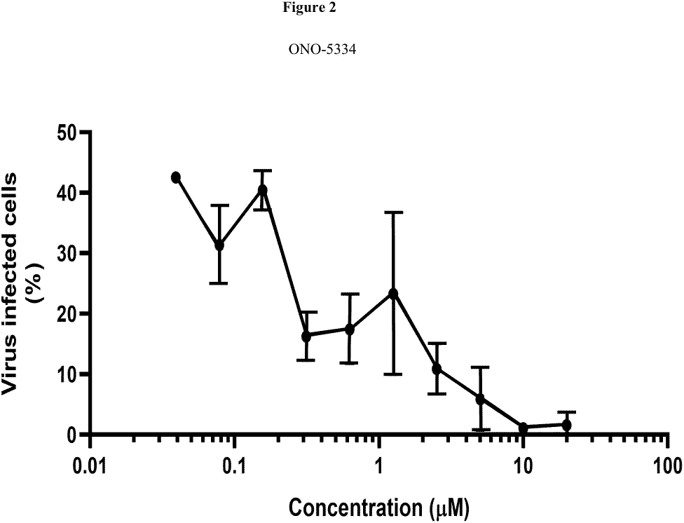

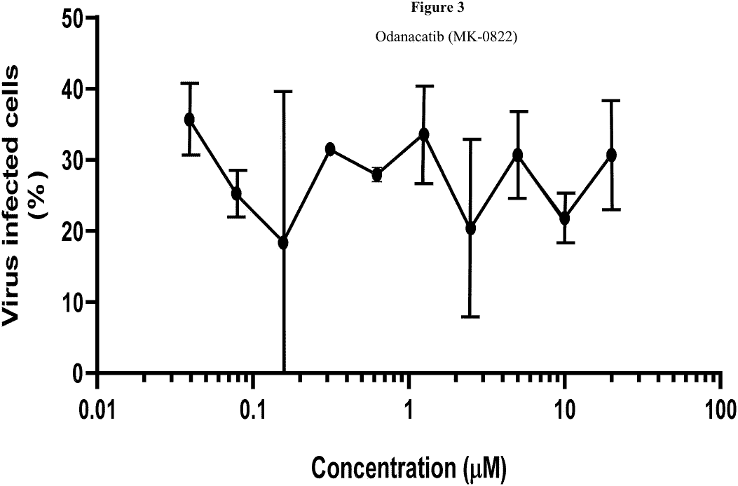

Robust antiviral efficacy of Balicatib (HB-121) with approximate EC₅₀ of 0.25 µM to 0.5 µM was observed. (See FIG. 1). Based on human clinical dosing of Balicatib (HB-121), 400 mg oral dosing of Balicatib (HB-121) will result in plasma concentrations of Balicatib (HB-121) which is above EC₅₀ for 16-20 hrs. This suggests dosing of 400 mg orally once or twice daily can be appropriate to observe clinical benefit from Balicatib (HB-121) against SARS-CoV-2. FIG. 2 shows that ONO-5334 demonstrated antiviral activity. FIG. 3 shows the percentage of virus infected cells treated with varying concentrations of Odanacatib (MK-0822). There was no drug dose dependent antiviral activity observed for Odanacatib.

### Example 3. Evaluation Assay for SARS-CoV

Many negative and positive strand RNA viruses must bind, fuse, enter, and use host proteins to be able to productively infect human cells. Many negative strand RNA viruses such as Ebola, Marburg, Nipah, SARS, SARS-CoV-2, and MERS use cathepsin inhibitors to enter and replicate within human cells. Some viruses such as SARS-CoV-2 have their own proteases which allows them to invade host detection or use their proteases to cleave their proteins. However, these proteases alone are not able to sustain cellular infection and the virus must use host protases to be able to infect and replicate within human cells. SARS-CoV-2 has at least two essential proteases and at least one cleavage site in its spike protein for cathepsins. There are several cell proteases/cathepsins that are essential for SARS-CoV-2 productive replication.

Proteases such as cysteine cathepsins can required for viral replication and inhibition of these cathepsins with cysteine cathepsin inhibitor such as Balicatib protects against SARS-CoV-2 infection. An objective is to test clinically advanced cathepsin inhibitors against SARS-CoV-2 infection in relevant human cells and obtaining EC₅₀/₉₀ and determining the best drug-like advanced candidate for repurposing against SARS-CoV-2 infection. This hypothesis is supported by the data for Balicatib, a cathepsin inhibitor which has been in phase 2 clinical trial for osteoporosis. Balicatib (HB 0121) acts a potent antiviral against SARS-CoV-2 with an effective concentration (EC) which inhibits 50% of SARS-CoV-2 infection in nM range. Balicatib (HB 0121) has a (SI) and cell cytotoxicity (CC) profile as shown below. The data shows that Balicatib has a SI of >300 and CC₅₀ of over 100 µM in Calu-3 human cells. Based on these data and Balicatib’s pharmacokinetics in human after a single dose of 5, 50, or 400 mg, the calculated adult human dose is about 50 mg-3,600 mg daily dosing. Clinical dosing can be further refined based on the exact EC₅₀ and EC₉₀ of Balicatib against SARS-CoV-2.

Balicatib was purchased from MedChemExpress (www.medchemexpress.com). The vial contained 5 mg of Balicatib (HB 0121) the compound was over 97% pure and was dissolved in DMSO to obtain a 10mM concentration. The efficacy of Balicatib was determined in two separate studies. In study one 0.1, 1, and 10 µM final concentration was tested and in study two six doses’ concentrations 0.1, 0.3, 1, 3, 10, and 30 µM of Balicatib (HB 0121) was examined.

Below is the protocol that was used to test the efficacy of Balicatib in 6 doses against SARS-CoV-2 using Calu-3 cells. The study number 1, Balicatib (HB 0121) was diluted to 20 µM and then 1:10 serial dilutions to achieve 2 µM, 0.2 µM. The rest of the study was similar as outlined below. Calu-3 Cells were seeded at 2e5cells/well in a 24 well plates in the BSL-2 lab the previous day (cells are at passage 6). Preparation of HB 0121 (Balicatib) inhibitor stock for the experiment: the inhibitor stock vial HB 0121 was prepared at 10 mM concentration in DMSO. The inhibitor was tested at concentrations of 30 uM, 10 uM, 3 uM, 1 uM, 0.3 uM and 0.1 uM.

The stock concentrations was prepared according to the following steps: 10 mM stock: 10 ul + 90 ul of direct EMEM media without FBS = 1 mM stock; 1 mM stock: 150 ul + 1.35 ml of direct EMEM media without FBS= 100 uM stock; 100 uM stock: 900 ul + 600 ul of direct EMEM media without FBS= 60 uM stock; 100 uM stock: 300 ul + 1.2 ml of direct EMEM media without FBS= 20 uM stock; 60 uM stock: 150 ul + 1.35 ml of direct EMEM media without FBS= 6 uM stock; 20 uM stock: 150 ul + 1.35 ml of direct EMEM media without FBS= 2 uM stock; 6 uM stock: 150 ul + 1.35 ml of direct EMEM media without FBS= 0.6 uM stock; 2 uM stock: 150 ul + 1.35 ml of direct EMEM media without FBS= 0.2 uM stock.

The experiment was set up as follows (8 Wells total): 1) Control well (no infection and no treatment); 2) Control well treated with drug diluent (DMSO at the same final conc. as 30 uM treatment); 3) Inhibitor at 30 uM concentration with infection; 4) Inhibitor at 10 uM concentration with infection; 5) Inhibitor at 3 uM concentration with infection; 6) Inhibitor at 1 uM concentration with infection; 7) Inhibitor at 0.3 uM concentration with infection; 8) Inhibitor at 0.1 uM concentration with infection.

All reagents and the plate were taken into the BSC hood. The cells were pretreated with 500 µl of the preparation for each corresponding labeled well. The plates were placed inside the 37° C./5% CO2 incubator and allowed to incubate for 4 hrs. After the incubation time, the plate was taken into the BSC and the contents of each well were removed and stored in a separate tube as backup. The cells were infected with SARS-CoV-2 virus (obtained from ATCC/BEI resources Washington strain) at MOI of 0.1 in 100 ul of EMEM containing 10% FBS (original titer of stock virus is 4.5e6/ml; so, 4.44 ul of the virus stock will be added to each well to achieve 0.1 MOI). The plates were transferred to 37° C. /5% CO₂ incubator for 1 hr (rock plate gently every 10 minutes for even distribution of virus). The plates were taken back into the BSC and the virus was removed. The discarded virus was bleached based on GMU SOP. Cells were washed two times with PBS. Mixture of 500 ul each of the above inhibitor mixed with 500 ul of EMEM containing 20% FBS were prepared and added to each of the corresponding wells. The plates were placed in 37° C./5% CO₂ incubator and allowed to incubate for 72 hrs. The cells were observed daily for CPE, and their viability were measured at the 72 hour time point. At 72 hours, the supernatants were collected and spun down at 1200 × g for 5 minutes to remove floating cells, and the remaining supernatants were used for plaque assay analysis. The residual cells, pellets, and other tissue culture reagents were bleached per GMU SOP.

### Method: Plaque Assay Protocol for SARS-CoV-2

The reagents for the plaque assay include one or more cell culture media. Complete EMEM+++++ media was prepared using 1× bottle 2× EMEM for plaques (500 ml), 10% FBS (50 mL), 1% Minimum Essential Amino Acids (NEAA) (5 ml), 1% Sodium Pyruvate (5 ml), 1% L-glutamine (200 mM) (5 ml), 1% Pen/Strep (5 ml). Complete DMEM+++ media was prepared using 1× bottle Dulbecco’s modified eagle medium (DMEM) (500 mL), 1× 25 mL aliquot FBS (5%), 1× L-glutamine (200 mM) (5 ml), 1× Pen/Strep (5ml).

Crystal violet solution was prepared using 1% Crystal violet, 20% Ethanol, 79% dH2O, Agarose (0.6%) and 0.6 g in 100 mL of H2O.

On day one, cells were prepared. The day before the assay, 2.5×105 Vero cells/mL were seeded in a 12 well plate and incubated at 37° C./5% CO2 in order to achieve a 90 -100% confluency the following day. On day 2, samples were prepared. The confluency and health of the Vero cells was checked before starting the assay. Ten-fold dilutions of each test sample in DMEM was performed using deep-well 96 well plates (sample undiluted testing was included as needed). 450 µl DMEM was added to all wells. 50 µl of each test sample was added to the well in the first row containing DMEM and mixed the content of the well by pipetting up and down multiple times. The pipet tips were changed and 50 µl was transferred from the first dilution wells in the first row to the next row of wells using a multichannel pipette. The samples were mixed, the pipette tips were changed and the process was repeated at this step until all desired dilutions are prepared.

On day 3, the infection was carried out. Media was aspirated from the 12 well-plate, and 200 µl of each test sample dilution was added to two separate wells containing the Vero cell monolayer (to prepare technical duplicates). To prevent wells from drying up while all the sample dilutions were added, aspirated-2-4 wells at a time. The plate was incubated for 1 hr at 37° C./5%CO2, rocking it gently every 10-15 mins to prevent drying in the center. The overlay was prepared. A 1:1 mixture of 0.6% agarose in diH2O and EMEM was prepared. The agarose was heated on a hot plate or in a microwave until it melted and was cooled down in the water bath to about 56° C. The agarose was added to cold EMEM so that the mixture could reach a temperature below 50° C. It is essential not to add the overlay to the cells when it is hotter than 50° C. or the cells will die.

Further, provided 100 ul of HB 0121 in DMSO at 10 mM concentration was provided. The investigator was blinded to name of the compound to reduce any potential investigator bias on the outcome of the studies. Balicatib (HB 0121) was added to cells at various concentrations and after a few hours of incubation the SARS-CoV-2 was added. The infection was stopped at 72 hours and the amount of infection was recorded based on plaque formation. The control sample contained the highest amounts of DMSO which was used to dilute Balicatib and was used as “control”.

Once the infection was complete, the plates were checked the plates under microscope again. 0.8-1 mL of the agarose overlay and incubated at 37° C./5% CO2 for 2 days. The plates were not shaken or agitated during this period to avoid getting smudged plaques.

The cells were fixed with 10% formaldehyde in diH2O (~1 mL per well) using a pasture pipet and left for 1 hour inside the chemical hood. The formaldehyde was then gently removed using a pasture pipet, making sure not to touch the cells with the pipet tip. The formaldehyde was discarded in the appropriate container. The plate was inverted to expel out the overlay onto a sheet of paper towel inside the sink. A few drops of crystal violet were added to each well and allowed to sit for 5 mins. Using a pasture pipet, added enough diH2O to each well to wash out excess crystal violet. Extra washes could be done if needed.

The plaques in each well were counted, taking the average of technical replicates of the same dilution: The plaques were counted the plaques in each well, taking the average of technical replicates of the same dilution. Pfu/ml was calculated as the average number of plaques divided by the product of dilution (D) and volume of diluted virus added to the plate. After the number of plaques was determined for control and experimental treatments, the following formula was used to obtain % inhibition of the drug. The results are shown in Table 5 and Table 6.

$$\text{\%} \text{inhibition} \text{=} \begin{matrix}\# \text{of plaques from treatment} \\ \text{------------------------------------------ X} 100 \\ \# \text{of plaques from control}\end{matrix}$$

TABLE 5

Antiviral activity of Balicatib (HB 0121) against SARS-CoV-2 infection using human lung cells (Calu-3 cells)

| | % Inhibition | % Inhibition | Overall % Inhibition (average of two experiments) |
|---|---|---|---|
| Concentration | Experiment 1 | Experiment 2 | Combined |
| 100 nM | 97.3 | 18 | 57.75 |
| 300 nM | NA | 90 | 90 |
| 1 µM | 64 | 72.5 | 68.25 |
| 3 µM | NA | 100 | 100 |
| 10 µM | 98.8 | 100 | 99.4 |
| 30 µM | NA | 100 | 100 |
| DMSO Control | 4750 pfu | 28000 pfu | NA |

In Table 6, Cell Appearance: No CPEs (no clumping, cell swelling or shrinkage, and no cell detachment). Cells were seeded at 2.0E5 per well of a 24-well plate in a 1 ml volume of media. Cells were at 98.7% viability at seeding.

TABLE 6

Effect of Balicatib (HB 0121) at 100 µM on Calu 3 cell viability and proliferation

| Sample | Count (cells/ml) at 24 hr time point | Viability (%) at 24 hr time point |
|---|---|---|
| Control #1 (no treatment) | 1.92e5 | 90.8 |
| Control #2 (no treatment) | 2e5 | 76.1 |
| 100 µM (#1) | 1.8e5 | 71.0 |
| 100 µM (#2) | 1.6e5 | 72.1 |

The data clearly shows that Balicatib (HB 0121) is a SARS-CoV-2 antiviral at nM concentrations, as shown in Table 5 and Table 6.

Table 5 depicts the data obtained from two experiments in which cells were treated with Balicatib (HB 0121) and infected with SARS-CoV-2 virus at MOI (Multiplicity of Infection) of 0.1. The data clearly shows that Balicatib (HB 0121) inhibited the replication of SARS-CoV-2. Balicatib (HB 0121) inhibited almost 100% of the viral replication at 3, 10, and 30 uM. At and below 1 µM the efficacy of Balicatib was reduced but was substantially different than control. The efficacy, as measured by % inhibition of SARS-CoV-2 replication of Balicatib (HB 0121) was about 68% and 57% at 1 µM and 100 nM, respectively. Hence, based on these two studies, it is asserted that the EC50 of Balicatib is in low to high nM range (50-600 nM) and EC₉₀ is in very low uM range (1-3 µM). Because in this study Balicatib (HB 0121) was not dosed down enough, below 100 nM, a clear EC₅₀ was not reached. In future studies, Balicatib (HB 0121) can be reduced to 10 nM.

These studies were performed by plaque assay, which is the “gold standard” for analyzing efficacy of antivirals. The plaque assay examines actual infection. Further, the size of the plaque, shape of the plaque, and the quality of the plaques allows one to better understand the quality of antiviral activity of the compounds. However, the plaque assay is difficult to perform and is within 0.5 log accuracy and reproducibility. Therefore, it is not unusual to see differences between several replicate studies, specially at the low end of the effective concentrations of antivirals. Here, at lower concentration of Balicatib (HB0121), the data was variable between the two studies specifically at the 100-300 nM. To attempt to decrease variation between studies, Balicatib (HB 0121) will be tested against SARS-CoV-2 and other viruses that use cathepsins for entry and replication by various methods such as QTRPCR and immunostaining or high-content imaging. By using other technologies to test Balicatib against SARS-CoV-2 an opportunity exists to better define Balicatib’s EC₅₀ and EC₉₀. Table 5 shows that Balicatib does not alter cellular viability and it has little to no effect on cell growth. Based on these data Selectivity Index (SI) and Cell Cytotoxicity for Balicatib were calculated to be at >300 and >100 µM, respectively.

The above method (plaque assay) was used to perform antiviral testing for ONO-5334 (HB 0119) and Odanacatib (HB 0122) against SARS-CoV-2 infection using human lung cells (Calu-3 cells). The data shows that ONO-5334 (HB 0119) has EC₅₀ value of less than 1 µM and EC₉₀ is between 1-10 µM, as shown in Table 7. Odanacatib (HB 0122) another cysteine cathepsin inhibitor has lower activity against SARS-CoV-2 infection using Calu-3 cells. This antiviral has EC₅₀ of about 1 µM and EC₉₀ 1-10 µM. More studies are being performed to fully define EC₅₀ and EC₉₀ of these compounds by various methods such as QRTPCR and high content imaging. The data clearly show that both of these compounds are antiviral against SARS-CoV-2 and can be useful for pre or post exposure to the virus in clinical settings.

TABLE 7

Antiviral activity of ONO-5334 (HB 0119) and Odanacatib (HB 0122) against SARS-CoV-2 infection using human lung cells (Calu-3 cells)

| | ONO-5334 | Odanacatib |
|---|---|---|
| Concentration | % Inhibition | % Inhibition |
| 1 µM | 79 | 48.4 |
| 10 µM | 100 | 97.1 |

### Example 4. Testing for Synergistic Compounds

The disclosure provides methods for the identification of a compound that produces synergistic activity with a drug of choice. In certain aspects, the disclosure provides methods for the identification of a compound that reduces the effective dosage of a drug of choice. Any technique well-known to the skilled artisan can be used to screen for a compound that would reduce the effective dose of a drug. As an example, a cell is contacted with a test mammalian protease inhibitor in combination with a drug of choice, for example an antiviral drug. A control without the test mammalian protease inhibitor is provided. The cell can be contacted with a test mammalian protease inhibitor before, concurrently with, or subsequent to the administration of the drug. In certain embodiments, the cell is incubated with multiple concentrations of the drug and test mammalian protease inhibitor, for at least 1 minute to at least during the experiment. The effect of the combination on the viral replication is measured at any time during the assay. In certain embodiments, a time course of viral replication in the culture is determined. If the viral replication is inhibited or reduced in the presence of the test compound at reduced drug concentrations wherein the effect is more than an additive effect, the test mammalian protease inhibitor is identified as being effective in producing a synergistic activity.

### Example 5. Assays for Evaluating the Combinations

The combinations of the disclosure can be tested for in vitro activity against a disease or microorganism and sensitivity, and for cytotoxicity in laboratory adapted cell lines or cultured cells such as peripheral blood mononuclear cells (PBMC), human fibroblast cells, hepatic, renal, epithelium cells, according to standard assays developed for testing compounds. Combination assays can be performed at varying concentrations of the compounds of the combinations to determine EC 50 by serial dilutions.

### In Vitro Assays

Cells: HEp-2 (CCL-23), PC-3 (CCL-1435), HeLa (CCL-2), U2OS (HTB-96), Vero (CCL-81), HFF-1 (SCRC- 1041), Calu-3, Hela cells with human ACE-2, Hu7.5 cells, MRC5 cells and HepG2 (HB-8065) cell lines can be purchased from the American Type Culture Collection. HEp-2 cells can be cultured in Eagle’s Minimum Essential Media (MEM) with GlutaMAX supplemented with 10% fetal bovine serum (FBS) and 100 U ml-1 penicillin and streptomycin. PC-3 cells can be cultured in Kaighn’s F12 media supplemented with 10% FBS and 100 U ml-1 penicillin and streptomycin. HeLa, U2OS, and Vero cells can be cultured in MEM supplemented with 10% FBS, 1% L-glutamine, 10 mM HEPES, 1% non-essential amino acids, and 1% penicillin/streptomycin. HFF-1 cells can be cultured in MEM supplemented with 10% FBS and 0.5 mM sodium pyruvate. HepG2 cells can be cultured in Dulbecco’s Modified Eagle Medium (DMEM) with GlutaMAX supplemented with 10% FBS, 100 U ml-1 penicillin and streptomycin, and 0.1 mM non-essential amino acids. The MT-4 cell line can be obtained from the NIH AIDS Research and Reference Reagent Program and cultured in RPMI-1640 medium supplemented with 10% FBS, 100 U ml-1 penicillin and streptomycin, and 2 mM L glutamine. The Huh-7 cell line can be obtained from C. M. Rice (Rockefeller University) and cultured in DMEM supplemented with 10% FBS, 100 U ml-1 penicillin and streptomycin, and non-essential amino acids. Primary human hepatocytes or other primary cell can be purchased from Invitrogen and cultured in William’s Medium E medium containing cell maintenance supplement. Donor profiles will be limited to 18- to 65-year-old nonsmokers with limited alcohol consumption. Upon delivery, the cells will be allowed to recover for 24 h in complete medium with supplement provided by the vendor at 37° C. Human PBMCs will be isolated from human buffy coats obtained from healthy volunteers (Stanford Medical School Blood Center, Palo Alto, California) and maintained in RPMI-1640 with GlutaMAX supplemented with 10% FBS, 100 U ml-1 penicillin and streptomycin. To test viral inhibition in primary nonhuman primate cells, Rhesus fresh whole blood will be obtained from Valley Biosystems or other suppliers. PBMCs will be isolated from whole blood by Ficoll Hypaque density gradient centrifugation. Briefly, blood will be overlaid on 15 ml Ficoll-Paque (GE Healthcare Bio-Sciences AB), and centrifuged at 500 g for 20 min. The top layer containing platelets and plasma will be removed, and the middle layer containing PBMCs will be transferred to a fresh tube, diluted with Tris buffered saline up to 50 ml, and centrifuged at 500 g for 5 min. The supernatant will be removed and the cell pellet will be resuspended in 5 ml red blood cell lysis buffer (155 mM ammonium chloride, 10 mM potassium bicarbonate, 0.1 mM EDTA, pH 7.5). To generate stimulated PBMCs, freshly isolated quiescent PBMCs will be seeded into a T-150 (150 cm2) tissue culture flask containing fresh medium supplemented with 10 U ml-1 of recombinant human interleukin-2 (IL-2) and 1 µg ml-1 phytohaemagglutinin-P at a density of 2 × 10⁶ cells ml-1 and incubated for 72 h at 37° C. Human macrophage cultures will be isolated from PBMCs that will be purified by Ficoll gradient centrifugation from 50 ml of blood from healthy human volunteers. PBMCs will be cultured for 7 to 8 days in in RPMI cell culture media supplemented with 10% FBS, 5 to 50 ng ml-1 granulocyte-macrophage colony-stimulating factor and 50 µM β- mercaptoethanol to induce macrophage differentiation. The cryopreserved human primary renal proximal tubule epithelial cells will be obtained from LifeLine Cell Technology and isolated from the tissue of human kidney. The cells will be cultured at 90% confluency with RenaLife complete medium in a T-75 flask for 3 to 4 days before seeding into 96-well assay plates. Immortalized human microvascular endothelial cells (HMVEC-TERT) will be obtained from R. Shao at the Pioneer Valley Life Sciences Institute. HMVEC-TERT cells will be cultured in endothelial basal media supplemented with 10% FBS, 5 µg of epithelial growth factor, 0.5 mg hydrocortisone, and gentamycin/amphotericin-B. In some experiments it can be essential to evaluate the intracellular metabolism (phosphorylation) of nucleobase and nucleoside (Nuc) these studies will be performed as below. The intracellular metabolism of nucleoside will be assessed in different cell types (HMVEC and HeLa cell lines, and primary human and rhesus PBMCs, monocytes and monocyte-derived macrophages) following 2-h pulse or 72-h continuous incubations with 10-1,000 µM of nucleobase or nucleoside. For comparison, intracellular metabolism during a 72-h incubation with 10-1,000 µM of Nuc will be completed in human monocyte-derived macrophages. For pulse incubations, monocyte-derived macrophages isolated from rhesus monkeys or humans will be incubated for 2h in compound-containing media followed by removal, Uimethylhexylamine (DMH) in water for analysis by liquid chromatography coupled to triple quadrupole mass spectrometry (LC-MS/MS). LC-MS/MS will be performed using low-flow ion pairing chromatography, similar to methods described previously (Durand-Gasselin L, et al. Nucleotide analogue prodrug tenofovir disoproxil enhances lymphoid cell loading following oral administration in monkeys. Mol. Pharm. 2009; 6:1145-1151). Briefly, analytes will be separated using a 50 × 2 mm × 2.5 µm Luna C18(2) HST column (Phenomenex) connected to a LC-20ADXR (Shimadzu) ternary pump system and HTS PAL autosampler (LEAP Technologies). A multi-stage linear gradient from 10% to 50% acetonitrile in a mobile phase containing 3 mM ammonium formate (pH 5.0) with 10 mM dimethylhexylamine over 8 min at a flow rate of 150 µl min-1 will be used to separate analytes. Detection will be performed on an API 4000 (Applied Biosystems) MS/MS operating in positive ion and multiple reaction monitoring modes. Intracellular metabolites alanine metabolite, Nuc, nucleoside monophosphate, nucleoside diphosphate, and nucleoside triphosphate will be quantified using 7- point standard curves ranging from 0.274 to 200 pmol (approximately 0.5 to 400 µM) prepared in cell extract from untreated cells. Levels of adenosine nucleotides will be also quantified to assure dephosphorylation had not taken place during sample collection and preparation. In order to calculate intracellular concentration of metabolites, the total number of cells per sample will be counted using a Countess automated cell counter (Invitrogen).

### Ebola Antiviral Testing Using Huh-7 and HMVEC

Antiviral assays can be conducted in a biosafety level 4 containment (BSL-4), for example at the Centers for Disease Control and Prevention. EBOV antiviral assays will be conducted in primary HMVEC-TERT and in Huh-7 cells. Huh-7 cells will not be authenticated and will not be tested for mycoplasma. Ten concentrations of compound will be diluted in fourfold serial dilution increments in media, and 100 µl per well of each dilution will be transferred in duplicate (Huh-7) or quadruplicate (HMVEC-TERT) onto 96-well assay plates containing cell monolayers. The plates will be transferred to BSL-4 containment, and the appropriate dilution of virus stock will be added to test plates containing cells and serially diluted compounds. Each plate will include four wells of infected untreated cells and four wells of uninfected cells that serve as 0% and 100% virus inhibition controls, respectively. After the infection, assay plates will be incubated for 3 days (Huh-7) or 5 days (HMVEC-TERT) in a tissue culture incubator. Virus replication will be measured by direct fluorescence using a Biotek HTSynergy plate reader. For virus yield assays, Huh- 7 cells will be infected with wild-type EBOV for 1 h at 0.1 plaque-forming units (PFU) per cell. The virus inoculum will be removed and replaced with 100 µl per well of media containing the appropriate dilution of compound. At 3 days post-infection, supernatants will be collected, and the amount of virus will be quantified by endpoint dilution assay. The endpoint dilution assay will be conducted by preparing serial dilutions of the assay media and adding these dilutions to fresh Vero cell monolayers in 96-well plates to determine the tissue culture infectious dose that caused 50% cytopathic effects (TCID₅₀). To measure levels of viral RNA from infected cells, total RNA will be extracted using the MagMAX-96 Total RNA Isolation Kit and quantified using a quantitative reverse transcription polymerase chain reaction (qRTPCR) assay with primers and probes specific for the EBOV nucleoprotein gene. Assay in HeLa and HFF-1 cells Antiviral assays will be conducted in BSL-4. HeLa or HFF-1 cells will be seeded at 2,000 cells per well in 384-well plates. Ten serial dilutions of compound in triplicate will be added directly to the cell cultures using the HP D300 digital dispenser (Hewlett Packard) in twofold dilution increments starting at 10 µM at 2 h before infection. The DMSO concentration in each well will be normalized to 1% using an HP D300 digital dispenser. The assay plates will be transferred to the BSL-4 suite and infected with EBOV Kikwit at a multiplicity of infection of 0.5 PFU per cell for HeLa cells and with EBOV Makona at a multiplicity of infection of 5 PFU per cell for HFF-1 cells. The assay plates will be incubated in a tissue culture incubator for 48 h. Infection will be terminated by fixing the samples in 10% formalin solution for an additional 48 h before immunostaining, as described.

### EBOV Human Macrophage Infection Assay

Antiviral assays will be conducted in BSL-4. Primary human macrophage cells will be seeded in a 96-well plate at 40,000 cells per well. Eight to ten serial dilutions of compound in triplicate will be added directly to the cell cultures using an HP D300 digital dispenser in threefold dilution increments 2 h before infection. The concentration of DMSO will be normalized to 1% in all wells. The plates will be transferred into the BSL-4 suite, and the cells will be infected with 1 PFU per cell of EBOV in 100 µl of media and incubated for 1 h. The inoculum will be removed, and the media will be replaced with fresh media containing diluted compounds. At 48 h post infection, virus replication will be quantified by immuno-staining.

### RSV A2 Antiviral Assay

For antiviral tests, compounds will be threefold serially diluted in source plates from which 100 mL. of diluted compound will be transferred to a 384-well cell culture plate using an Echo acoustic transfer apparatus. HEp-2 cells will be added at a density of 5 × 10⁵ cells per ml, then infected by adding RSV A2 at a titer of 1 × 10⁴ tissue culture infectious doses (TCID₅₀) per ml. Immediately following virus addition, 20 µl of the virus and cells mixture will be added to the 384-well cell culture plates using a µFlow liquid dispenser and cultured for 4 days at 37° C. After incubation, the cells will be allowed to equilibrate to 25° C. for 30 min. The RSV-induced cytopathic effect will be determined by adding 20 µl of CellTiter-Glo Viability Reagent. After a 10-min incubation at 25° C., cell viability will be determined by measuring luminescence using an Envision plate reader.

### High Content Imaging Assay Detecting Viral Infection

Antiviral assays will be conducted in 384-or 96-well plates in BSL-4 using a high-content imaging system to quantify virus antigen production as a measure of virus infection. A ‘no virus’ control and a ‘1% DMSO’ control will be included to determine the 0% and 100% virus infection, respectively. The primary and secondary antibodies and dyes used for nuclear and cytoplasmic staining are listed. The primary antibody specific for a particular viral protein will be diluted 1,000-fold in blocking buffer (1 × PBS with 3% BSA) and added to each well of the assay plate. The assay plates will be incubated for 60 min at room temperature. The primary antibody will be removed, and the cells will be washed three times with 1 × PBS. The secondary detection antibody will be an anti-mouse (or rabbit) IgG conjugated with Dylight488 (Thermo Fisher Scientific, catalogue number 405310). The secondary antibody will be diluted 1,000-fold in blocking buffer and will be added to each well in the assay plate. Assay plates will be incubated for 60 min at room temperature. Nuclei will be stained using Draq5 (Biostatus) or 33342 Hoechst (ThermoFisher Scientific) for Vero and HFF-1 cell lines. Both dyes will be diluted in 1 × PBS. The cytoplasm of HFF-1 (EBOV assay) and Vero E6 (MERS assay) cells will be counter-stained with CellMask Deep Red (Thermo Fisher Scientific). Cell images will be acquired using a Perkin Elmer Opera confocal plate reader (Perkin Elmer) using a ×10 air objective to collect five images per well. Virus-specific antigen will be quantified by measuring fluorescence emission at a 488 nm wavelength and the stained nuclei will be quantified by measuring fluorescence emission at a 640 nm wavelength. Acquired images will be analyzed using Harmony and Acapella PE software. The Draq5 signal will be used to generate a nuclei mask to define each nucleus in the image for quantification of cell number. The CellMask Deep Red dye will be used to demarcate the Vero and HFF-1 cell borders for cell-number quantitation. The viral-antigen signal will be compartmentalized within the cell mask. Cells that exhibited antigen signal higher than the selected threshold will be counted as positive for viral infection. The ratio of virus positive cells to total number of analyzed cells will be used to determine the percentage of infection for each well on the assay plates. The effect of compounds on the viral infection will be assessed as percentage of inhibition of infection in comparison to control wells. The resultant cell number and percentage of infection will be normalized for each assay plate. Analysis of dose- response curve will be performed using GeneData Screener or similar software applying Levenberg- Marquardt algorithm for curve-fitting strategy. The curve-fitting process, including individual data point exclusion, will be pre-specified by default software settings. R2 value quantified goodness of fit and fitting strategy will be considered acceptable at R2 > 0.8.

### Virus Assays

All virus infections will be quantified by immuno-staining using antibodies that recognized the relevant viral glycoproteins.

### Marburg Virus Assay

HeLa cells will be seeded at 2,000 cells per well in a 384-well plate, and compounds will be added to the assay plates. Assay plates will be transferred to the BSL-4 suite and infected with 1 PFU per cell MARV, which resulted in 50% to 70% of the cells expressing virus antigen in a 48-h period.

### Sudan Virus Assay

HeLa cells will be seeded at 2,000 cells per well in a 384-well plate, and compounds will be added to the assay plates. Assay plates will be transferred to the BSL-4 suite and infected with 0.08 PFU SUDV per cell, which resulted in 50% to 70% of the cells expressing virus antigen in a 48-h period.

### Junín Virus Assay

HeLa cells will be seeded at 2,000 cells per well in a 384-well plate, and compounds will be added to the assay plates. Assay plates will be transferred to the BSL-4 suite and infected with 0.3 PFU per cell JUNV, which resulted in ~50% of the cells expressing virus antigen in a 48-h period.

### Lassa Fever Virus Assay

HeLa cells will be seeded at 2,000 cells per well in a 384-well plate, and compounds will be added to the assay plates. Assay plates will be transferred to the BSL-4 suite and infected with 0.1 PFU per cell LASV, which resulted in >60% of the cells expressing virus antigen in a 48-h period.

### Middle East Respiratory Syndrome, SARS-CoV1, and SARS-CoV-2 Assays

African green monkey (Chlorocebus sp.) kidney epithelial cells (Vero E6) will be seeded at 4,000 cells per well in a 384-well plate, and compounds will be added to the assay plates in a dose dependent manner. Assay plates will be transferred to the BSL-¾ suite and infected with 0.5 or other PFU per cell of MERS, SARS-CoV1 and 2 viruses, which resulted in >70% of the cells expressing virus antigen in a 48-h period.

### SARS-CoV-2 Infection Assay

Calu-3 cells (1 × 10⁴ cells) can be seeded in 96 well plate. After 24 h, cells will be washed and treated with inhibitor (s) in a serum-free medium (n=6). The incubation of cells with media alone will serve as a negative control (n=6). After 2-16 h of inhibitor(s) treatment, cells will be washed and replaced with serum-free media for infection with SARS-CoV-2 (strain: BEI_USA-WA1/2020) multiplicity of infection (MOI) of 0.01 for 1 h at 37° C. After infection, cells will be washed and replaced with 5% FBS containing media. After 48-72 h post-infection, cells will be fixed with 4% buffered paraformaldehyde for 15 min at room temperature. The fixed cells will be washed with PBS then permeabilized in 0.1% Triton X100 PBS solution for 15 min then blocked in 3% BSA PBS solution. The cells will be incubated with anti-S protein Rab (Sino Biological, PA, USA) at 1:1000 in the blocking solution overnight at 4° C., followed by incubation with 1:2000 diluted Alexa Fluor 488 conjugated secondary antibody (Thermo Fisher, MA, USA) for 1 h at room temperature. The cells will be counterstained for nuclei with Hoechst 33342 (Thermo Fisher, MA, USA). The fluorescent images will be captured by using a Nikon Eclipse Ts2R fluorescent microscope or other plate readers such as Operetta or Opera. Total and virus-infected cells will be counted by using Nikon NIS-Elements D software. High content imaging assays will be performed as described herein.

### Chikungunya Virus Assay

U2OS cells will be seeded at 3,000 cells per well in a 384-well plate, and compounds will be added to the assay plates. Assay plates will be transferred to the BSL suite and infected with 0.5 PFU per cell of CHIK, which resulted in >80% of the cells expressing virus antigen in a 48-h period.

### Venezuelan Equine Encephalitis Virus Assay

HeLa cells will be seeded at 4,000 cells per well in a 384-well plate, and compounds will be added to the assay plates. Assay plates will be transferred to the BSL-4 suite and infected with 0.1 PFU per cell VEEV, which resulted in >60% of the cells expressing virus antigen in a 20-h period.

### Cytotoxicity Assays

HEp-2 (1.5 × 10³ cells per well) and MT-4 (2 × 10³ cells per well) cells will be plated in 384- well plates and incubated with the appropriate medium containing threefold serially diluted compound ranging from 15 nM to 100,000 nM. PC-3 cells (2.5 × 10³ cells per well), HepG2 cells (4 × 10³ cells per well), hepatocytes (1 × 10⁶ cells per well), quiescent PBMCs (1 × 10⁶ cells per well), stimulated PBMCs (2 × 105 cells per well), and RPTEC cells (1 × 10³ cells per well) will be plated in 96- well plates and incubated with the appropriate medium containing threefold serially diluted compound ranging from 15 nM to 100,000 nM. Cells will be cultured for 4-5 days at 37° C. Following the incubation, the cells will be allowed to equilibrate to 25° C., and cell viability will be determined by adding Cell-Titer Glo viability reagent. The mixture will be incubated for 10 min, and the luminescence signal will be quantified using an Envision plate reader. Cell lines will be not authenticated and will be not tested for mycoplasma as part of routine use in cytotoxicity assays. For RSV, In vitro RSV RNA synthesis assay will be used RNA synthesis by the RSV polymerase will be reconstituted in vitro using purified RSV L/P complexes and an RNA oligonucleotide template (Dharmacon), representing nucleotides 1-14 of the RSV leader promoter. RNA synthesis reactions will be performed as described previously, except that the reaction mixture contained 250 µM guanosine triphosphate (GTP), 10 µM uridine triphosphate (UTP), 10 µM cytidine triphosphate (CTP), supplemented with 10 µCi [α- 32P]CTP, and either included 10 µM adenosine triphosphate (ATP) or no ATP. Under these conditions, the polymerase is able to initiate synthesis from the position 3 site of the promoter, but not the position 1 site. The NTP metabolite of GS-5734 will be serially diluted in DMSO and included in each reaction mixture at concentrations of 10, 30, or 100 µM as specified. RNA products will be analyzed by electrophoresis on a 25% polyacrylamide gel, containing 7 M urea, in Tris- taurine-EDTA buffer, and radiolabeled RNA products will be detected by autoradiography.

### RSV A2 Polymerase Inhibition Assay

Transcription reactions contained 25 µg of crude RSV RNP complexes in 30 µL of reaction buffer (50 mM Tris-acetate (pH 8.0), 120 mM potassium acetate, 5% glycerol, 4.5 mM MgCl2, 3 mM DTT, 2 mM EGTA, 50 µg ml-1 BSA, 2.5 U RNasin, 20 µM ATP, 100 µM GTP, 100 µM UTP, 100 µM CTP, and 1.5 µCi [α-32P]ATP (3,000 Ci mmol-1). The radiolabeled nucleotide used in the transcription assay will be selected to match the nucleotide analogue being evaluated for inhibition of RSV RNP transcription. To determine whether nucleotide analogues inhibited RSV RNP transcription, compounds will be added using a six-step serial dilution in fivefold increments. After a 90-min incubation at 30° C., the RNP reactions will be stopped with 350 µl of Qiagen RLT lysis buffer, and the RNA will be purified using a Qiagen RNeasy 96 kit. Purified RNA will be denatured in RNA sample loading buffer at 65° C. for 10 min and run on a 1.2% agarose/MOPS gel containing 2 M formaldehyde. The agarose gel will be dried, exposed to a Storm phosphorimaging screen, and developed using a Storm phosphorimager.

### Example 6. Viral Yield Reduction Assay Using A549-hACE2

A549-hACE2 cells were maintained using RPMI (Gibco) with 2 mM L-glutamine, 10% FBS and 1% P/S in 37° C. with 5% CO2. SARS-CoV-2 hCoV-19/USA/WA1/2020 virus was obtained from BEI Resources and amplified for master seed stocks by infection of Vero E6 at an MOI=0.1 with 2% FBS until cytopathology was observed in 70-80% of the cells. The virus was sequenced using next-generation sequencing using Artic primers.

One day prior to infection, 1 × 10⁵ A549-hACE2 cells were seeded in 1 mL cell culture medium (containing 5% FBS) per well in a 24-well plate. Next day, cells were washed once with 1 mL PBS. Then 0.5 mL media (RPMI+2%FBS+1%Penicillin/Streptomycin) and 0.5 mL of Balicatib were added into each well with final concentrations as 10, 5, 2.5, 1.25, 0.625, and 0.315 µM in triplicate. Virus alone and cell alone were included as controls. The plate was incubated for 1 hour at 37° C. After 1 hour incubation, cells were washed with 1X PBS, and virus was loaded as MOI=0.1. Plates were incubated for 1 h at 37° C., and rocked every 15 min. After incubation, virus was removed from wells and cells were washed again with PBS. Then, 1 mL of Balicatib in cell culture medium was added again. The plates were incubated for 24 hours at 37° C. with 5% CO2. The next day, supernatant from each well was collected for TCID₅₀.

The collected supernatant was subjected to a serial 10-fold dilutions that were added to Vero E6 cells that have been pre-seeded in 96-well plates. Each sample at each dilution were loaded in quadruplicates. After 72 hours incubation, 10 µL MTT solution was added into each well using a MultiFlo FX. After incubation for 2 hours at 37° C. with 5% CO2, 100 µL stop solution was added into each well, and absorbance at 570 nm of samples was measured using a EnVision Multimode plate reader. Conversion from absorbance value to virus positive/virus negative used the following formula: If (data point) ≥ (Cell Control)/2 value = 0, it indicates no virus. If (data point < (Cell Control)/2 value = 1, it indicates virus present. By plotting number of positive wells vs. log₁₀ dilutions of supernatant, a TCID₅₀ was calculated from 4 replicates of 8 dilutions in a 96-well plate.

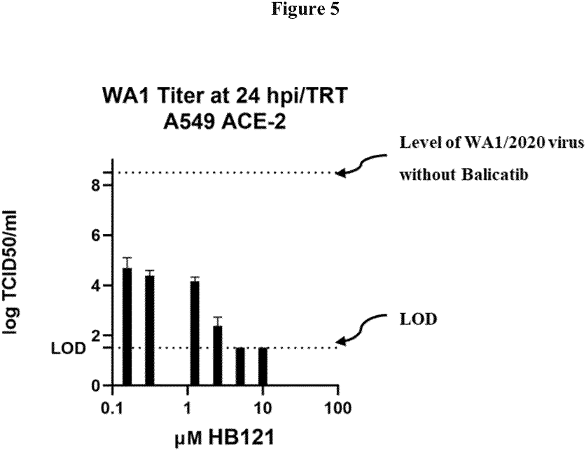

HB-121 resulted in a significant reduction of SARS-CoV-2 WA1/2020 in A549-hACE2 cells. Compared to well containing the virus but not Balicatib, treatment with 5 µM Balicatib (HB-121) treatment led to more than 6-log titer reduction in infected A549-hACE2 cells (FIG. 5). The lowest test concentration of Balicatib (HB-121) i.e., 0.3 µM of HB-121, caused more than a 3-log titer reduction in virus titer.

### Equivalents and Scope

Those skilled in the art will recognize, or be able to ascertain using no more than routine experimentation, many equivalents to the specific embodiments in accordance with the disclosure described herein. The scope of the present disclosure is not intended to be limited to the above Description, but rather is as set forth in the appended claims.

In the claims, articles such as “a,” “an,” and “the” can mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or the entire group members are present in, employed in, or otherwise relevant to a given product or process.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

In addition, it is to be understood that any particular embodiment of the present disclosure that falls within the prior art can be explicitly excluded from any one or more of the claims. Since such embodiments are deemed to be known to one of ordinary skill in the art, they can be excluded even if the exclusion is not set forth explicitly herein. Any particular embodiment of the compositions of the disclosure (e.g., any antibiotic, therapeutic or active ingredient; any method of production; any method of use; etc.) can be excluded from any one or more claims, for any reason, whether or not related to the existence of prior art.

It is to be understood that the words which have been used are words of description rather than limitation, and that changes can be made within the purview of the appended claims without departing from the true scope and spirit of the disclosure in its broader aspects.

While the present disclosure has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the disclosure.

1. A method of treatment of a coronavirus infection comprising administering to a subject in need thereof a mammalian protease inhibitor.

2. The method of claim 1, wherein the mammalian protease inhibitor has a structure of Formula (I): 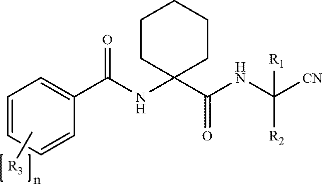
wherein,
R1 and R2 are independently H or C1-C7 lower alkyl, or R1 and R2 together with the carbon atom to which they are attached form a C3-C8 cycloalkyl ring;
R3 is an optionally substituted heterocyclic group comprising at least one nitrogen; and
n is between 1 and 3.

3. The method of claim 2, wherein the mammalian protease inhibitor has a structure of Formula (II): 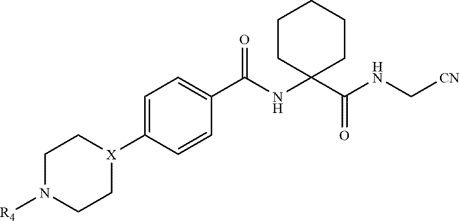
wherein X is CH or N; and
R4 is H, C1-C7 lower alkyl, C1-C7 lower alkoxy, C5-C10 aryl, or C3-C8 cycloalkyl.

4. The method of claim 1, wherein the mammalian protease inhibitor has a structure of: 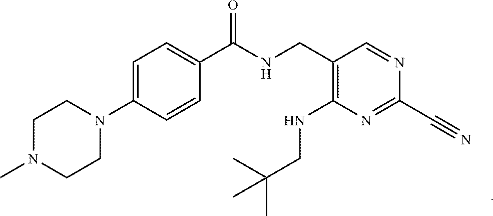
.

5. The method of claim 1, wherein the mammalian protease inhibitor has a structure of: 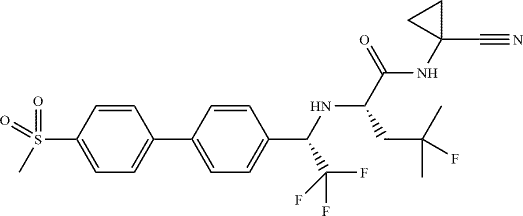 [SMILES: CC(C)(F)C[C@H](N[C@@H](c1ccc(-c2ccc(S(C)(=O)=O)cc2)cc1)C(F)(F)F)C(=O)NC1(C#N)CC1]
.

6. The method of claim 1, wherein the mammalian protease inhibitor is a cathepsin inhibitor comprising Balicatib, Odanacatib, Relacatib, a peptidyl aldehyde derivative, leupeptin, antipain, chymostatin, Ac-LVK-CH O, Z-Phe-Tyr-CHO, Z-Phe-Tyr(OtBu)-COCHO.H2O, 1-Naphthalenesulfonyl-Ile-Trp-CHO, Z-Phe-Leu-COCHO.H2O, a peptidyl semicarbazone derivative, a peptidyl methylketone derivative, peptidyl trifluoromethylketone, a biotin-Phe-Ala-fluoromethyl ketone, Z-Leu-Leu-Leu fluoromethyl ketone, Z-Phe-Phe-fluoromethyl ketone, N-Methoxysuccinyl-Phe-HOMO-Phe-fluoromethyl ketone, Z-Leu-Leu-Tyr-fluoromethyl ketone, Leupeptin trifluoroacetate, a peptidyl chloromethases, a peptidyl hydroxymate, a peptidylhydroxylamine, a peptidyl acyloxymethane, a peptidylacyloxymethyl ketone, a peptidyl aziridine , a peptidyl aryl vinylsufone , a peptidyl arylvinylsulfonate, a gallinamide analog, a peptidyl aldehyde , an azepinone-based inhibitor, a thiosemicarbazone, a propeptide mimic, a thiocarbazate, oxocarbazate, a peptidyl halomethylketone derivative, TLCK, a bis(acylamino) ketone, 1,3- Bis(CBZ-Leu-NH)-2-propanone, a peptidyl diazomethane, Z-Phe-Ala-CHN2, Z-Phe-Thr(OBzl)-CHN2, Z-Phe-Tyr (Ot-But)-CHN2, Z-Leu-Leu-Tyr-CHN2, a peptidyl methyl sulfonium salt, a peptidyl vinyl sulfone, LHVS, a peptidyl nitrile, a peptidyl disulfide, 5,5′-dithiobis[2-nitrobenzoic acid], cysteamines, 2,2′-dipyridyl disulfide, N-(4-Biphenylacetyl)-S-methyl cysteine-(D)-Arg-Phe-b phenethylamide, thiol alkylating agents, a maleimide, an azapeptide, an azobenzene, an O-acylhydroxamate, Z-Phe-Gly-NHO-Bz, Z-FG-NHO-BzOME, cystatin A, cystatin B, cystatin C, cystatin D, cystatin F, a stefin, Sialostain L, antimicrobial peptide LL-37, a procathepsin B fragment 26-50, a procathepsin fragment 36-50, SLV213, RO5459072, RWJ-445380, VBY036P1A, AM-3701, MIV-701, MIV-710, MIV-711, NC-2300, ORG-219517, ONO-5334, MK-0674, GB-111-NH2, L-873724, L-006235, AZD4996, VBY-036, RWY-445380, AM-3840, Cz-007, VBY-825, VBY-129, SAR-114137, VBY-891, Petesicatib, LY-3000328, MIV-247, CRA-028129, RG-7236, GSK2793660, BI-1181181, VBY-376, Begacestat, AL101 (BMS906024), BMS-986115 (AL-102), MK-0752 (L-000891675), EVP-0962 (EVP-0015962), SAR-164653, KGP94, VEL-0230, BLD2660, E-64, E-64a, E-64b, E-64c, E-64d, CA-074, CA-074 Me, CA-030, CA-028, chloroquine, ammonium chloride, or a derivative thereof.

7. (canceled)

8. The method of claim 6, wherein the cathepsin inhibitor is Balicatib and the concentration of Balicatib is about 0.1 µM to about 50 µM.

9-10. (canceled)

11. The method of claim 8, wherein the cathepsin inhibitor comprises an effective concentration (EC₅₀) of from about 0.25 µM to about 30 µM and/or an EC₉₀ of from about 1 µM to about 100 µM.

12-15. (canceled)

16. The method of claim 1, wherein administering to the subject the mammalian protease inhibitor inhibits replication of-a coronavirus by from about 50% to about 100%.

17. The method of claim 1, wherein the mammalian protease inhibitor comprises a selectivity index of at least 300.

18. The method of claim 16, wherein the coronavirus is a SARS-CoV-2 virus, a SARS-CoV-1 virus, a MERS-CoV virus, a 229E virus, a NL63 virus, a OC43 virus, a HKU1 virus, or variants thereof.

19-88. (canceled)

89. A composition comprising an antiviral drug and a mammalian protease inhibitor, wherein the antiviral drug is one or more of a nucleoside analog, a nucleotide analog, a viral polymerase inhibitor, a reverse transcriptase inhibitor, a viral envelope fusion inhibitor, a prophylactic agent, a protein drug, a proton transport inhibitor, or a neuraminidase inhibitor.

90. The composition of claim 89, wherein the mammalian protease inhibitor is a cathepsin inhibitor comprising Balicatib, Odanacatib, Relacatib, a peptidyl aldehyde derivative, leupeptin, antipain, chymostatin, Ac-LVK-CH O, Z-Phe-Tyr-CHO, Z-Phe-Tyr(OtBu)-COCHO.H2O, 1-Naphthalenesulfonyl-Ile-Trp-CHO, Z-Phe-Leu-COCHO.H2O, a peptidyl semicarbazone derivative, a peptidyl methylketone derivative, peptidyl trifluoromethylketone, a biotin-Phe-Ala-fluoromethyl ketone, Z-Leu-Leu-Leu fluoromethyl ketone, Z-Phe-Phe-fluoromethyl ketone, N-Methoxysuccinyl-Phe-HOMO-Phe-fluoromethyl ketone, Z-Leu-Leu-Tyr-fluoromethyl ketone, Leupeptin trifluoroacetate, a peptidyl chloromethases, a peptidyl hydroxymate, a peptidylhydroxylamine, a peptidyl acyloxymethane, a peptidylacyloxymethyl ketone, a peptidyl aziridine , a peptidyl aryl vinylsufone , a peptidyl arylvinylsulfonate, a gallinamide analog, a peptidyl aldehyde , an azepinone-based inhibitor, a thiosemicarbazone, a propeptide mimic, a thiocarbazate, oxocarbazate, a peptidyl halomethylketone derivative, TLCK, a bis(acylamino) ketone, 1,3- Bis(CBZ-Leu-NH)-2-propanone, a peptidyl diazomethane, Z-Phe-Ala-CHN2, Z-Phe-Thr(OBzl)-CHN2, Z-Phe-Tyr (Ot-But)-CHN2, Z-Leu-Leu-Tyr-CHN2, a peptidyl methyl sulfonium salt, a peptidyl vinyl sulfone, LHVS, a peptidyl nitrile, a peptidyl disulfide, 5,5′-dithiobis[2-nitrobenzoic acid], cysteamines, 2,2′-dipyridyl disulfide, N-(4-Biphenylacetyl)-S-methyl cysteine-(D)-Arg-Phe-b phenethylamide, thiol alkylating agents, a maleimide, an azapeptide, an azobenzene, an O-acylhydroxamate, Z-Phe-Gly-NHO-Bz, Z-FG-NHO-BzOME, cystatin A, cystatin B, cystatin C, cystatin D, cystatin F, a stefin, Sialostain L, antimicrobial peptide LL-37, a procathepsin B fragment 26-50, a procathepsin fragment 36-50, SLV213, RO5459072, RWJ-445380, VBY036P1A, AM-3701, MIV-701, MIV-710, MIV-711, NC-2300, ORG-219517, ONO-5334, MK-0674, GB-111-NH2, L-873724, L-006235, AZD4996, VBY-036, RWY-445380, AM-3840, Cz-007, VBY-825, VBY-129, SAR-114137, VBY-891, Petesicatib, LY-3000328, MIV-247, CRA-028129, RG-7236, GSK2793660, BI-1181181, VBY-376, Begacestat, AL101 (BMS906024), BMS-986115 (AL-102), MK-0752 (L-000891675), EVP-0962 (EVP-0015962), SAR-164653, KGP94, VEL-0230, BLD2660, E-64, E-64a, E-64b, E-64c, E-64d, CA-074, CA-074 Me, CA-030, CA-028, chloroquine, ammonium chloride, or a derivative thereof.

91-92. (canceled)

93. The composition of claim 89, wherein the nucleoside analog is T-705 (Favipiravir), BCX4430 (Galidesivir), Brincidofovir, FGE-106, JK-05, Triazavirin, Acyclovir Fleximer, Ribavirin, AL-335 (Adafosbuvir), 6-azauridine, gancyclovir, dideocycytidine, dideoxyinosine, GS-5734 (Remdesivir), JNJ-64041575, JNJ-1575, ALS-008176, AL-8176 (Lumicitabine), Hepsera (adefovir dipivoxil), Peveon, Viread (tenofovir disoproxil fumarate), Acycloadenosine, NITD008, MK-608, ribonucleoside analog β-d-N4-hydroxycytidine (NHC), EIDD-2801 (Molnupiravir), AT-527, AT-511, or resimiquid.

94-98. (canceled)

99. The composition of claim 89, wherein (i) the viral polymerase inhibitor is Foscarnet, Cidofovir, or Alovudine; (ii) the reverse transcriptase inhibitor is Nevirapine, Delavirdine, Efavirenz, Etravirine, Rilpivirine, Adefovir dipivoxil, or Atevirdine; (iii) the viral envelop fusion inhibitor is Docosanol, Enfuvirtide, or Maraviroc; (iv) the prophylactic agent is a vaccine selected from vRSV-IGIV, VZIG, or VariZIG; (v) the proton transport inhibitor is Rimantadine or Methisazone; or (vi) the neuraminidase inhibitor is Zanamivir, Oseltamivir, Laninamivir octanoate, or Peramivir.

100-117. (canceled)

118. A method of treatment or prophylaxis of a disease comprising administering to a subject in need thereof a composition of claim 89, wherein the disease is a viral infection that is caused by a Coronavirus, Orthomyxoviridae, influenza A virus, influenza B virus, influenza C virus, Thogotovirus, Dhori virus, infectious salmon anemia virus, Paramyxoviridae, parainfluenza virus, human respiratory syncytial virus (RSV), Sendai virus, Newcastle disease virus, mumps virus, rubeola (measles) virus, Hendra virus, Nipah virus, avian pneumovirus, canine distemper virus, Rhabdoviridae rabies virus, vesicular stomatitis virus (VSV), Mokola virus, Duvenhage virus, European bat virus, salmon infectious hematopoietic necrosis virus, viral hemorrhagic septicaemia virus, spring viremia of carp virus, snakehead rhabdovirus, Bornaviridae, Borna disease virus, Bunyaviridae Bunyamwera virus, Hantaan virus, Crimean Congo virus, California encephalitis virus, Rift Valley fever virus, sandfly fever virus, Arenaviridae Old World Arenaviruses, Lassa virus, Ippy virus, Lymphocytic choriomeningitis virus (LCMV), Mobala virus, Mopeia virus, New World Arenaviruses, Junin virus (Argentine hemorrhagic fever), Sabia (Brazilian hemorrhagic fever), Amapari virus, Flexal virus, Guanarito virus (Venezuela hemorrhagic fever), Machupo virus (Bolivian hemorrhagic fever), Latino virus, Boliveros virus, Parana virus, Pichinde virus, Pirital virus, Tacaribe virus, Tamiami virus, Whitewater Arroyo virus, arboviruses, togaviruses, Alphaviruses, Venezuela equine encephalitis virus, Sindbis virus, Rubivirus, Rubella virus, Chikungunya virus, Marburg viruses, Sudan virus, Flaviviridae, flavivirus, Pestivirus, and Hepacivirus, yellow fever virus, dengue fever virus, and Japanese encaphilitis (JE) virus, Pestivirus, Hepacivirus, hepatitis C virus, hepatitis C-like viruses, Japanese encephalitis Alfuy, Japanese encephalitis, Kokobera, Koutango, Kunjin, Murray Valley encephalitis, St. Louis encephalitis, Stratford, Usutu, West Nile viruses, Pestivirus, bovine viral diarrhea virus (BVDV), classical swine fever virus (CSFV), border disease virus (BDV), Arenaviridae, Lymphocytic choriomeningitis virus (LCMV), Phlebovirus Rift valley fever virus, Hendra, Nipah, Riboviria, a rhinovirus, an enterovirus, a poliovirus, or an adenovirus.

119. (canceled)

120. The method of claim 118, wherein the coronavirus is SARS-CoV-2, SARS-CoV-1, or MERS-CoV.

121-125. (canceled)

126. The composition of claim 89, wherein the mammalian protease inhibitor has a structure of Formula (I): 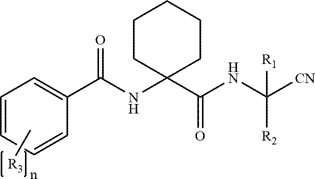
wherein,
R1 and R2 are independently H or C1-C7 lower alkyl, or R1 and R2 together with the carbon atom to which they are attached form a C3-C8 cycloalkyl ring;
R3 is an optionally substituted heterocyclic group comprising at least one nitrogen; and
n is between 1 and 3.

127. The composition of claim 89, the mammalian protease inhibitor has a structure of Formula (II): 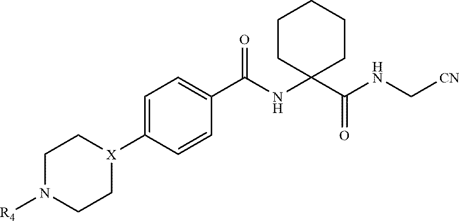
wherein X is CH or N; and
R4 is H, C1-C7 lower alkyl, C1-C7 lower alkoxy, C5-C10 aryl, or C3-C8 cycloalkyl.

128. The composition of claim 89, wherein the mammalian protease inhibitor has a structure of: 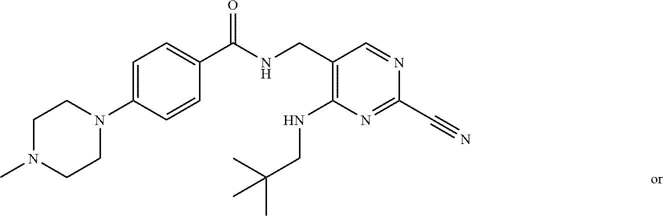 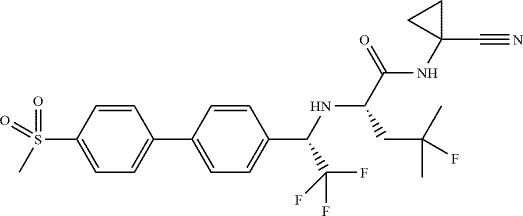 [SMILES: CC(C)(F)C[C@H](N[C@@H](c1ccc(-c2ccc(S(C)(=O)=O)cc2)cc1)C(F)(F)F)C(=O)NC1(C#N)CC1]
.
